(19) United States
(12) Patent Application Publication
(10) Pub. No.: US 2025/0136612 A1
(43) Pub. Date: May 1, 2025

(54) 4',5'-DIHYDROSPIRO[PIPERIDINE-4,7'-THIENO[2,3-C]PYRAN] DERIVATIVES AS INHIBITORS OF APOL1 AND METHODS OF USING SAME

(71) Applicant: VERTEX PHARMACEUTICALS INCORPORATED, Boston, MA (US)

(72) Inventors: Timothy J. SENTER, Arlington, MA (US); Samantha ANGLE, Cambridge, MA (US); Michael A. BRODNEY, Newton, MA (US); Jingrong CAO, Newton, MA (US); Jon COME, Cambridge, MA (US); Leslie A. DAKIN, Framingham, MA (US); Elena DOLGIKH, Boston, MA (US); Zachary GALE-DAY, Brookline, MA (US); Elaine B. KRUEGER, Milton, MA (US); Suganthini NANTHAKUMAR, Newton, MA (US); Jessica H. OLSEN, Jamaica Plain, MA (US); Akira J. SHIMIZU, Framingham, MA (US); Steven D. STONE, Quincy, MA (US); Haoxuan WANG, Somerville, MA (US)

(21) Appl. No.: 18/836,407

(22) PCT Filed: Feb. 8, 2023
(86) PCT No.: PCT/US2023/012578

§ 371 Date: Aug. 7, 2024

Related U.S. Application Data
(60) Provisional application No. 63/307,876, filed on Feb. 8, 2022.

(51) Int. Cl. C07D 495/20; A61K 31/438; A61K 31/444; A61K 31/497; A61K 31/501; A61K 31/506

(52) U.S. Cl. CPC C07D 495/20; A61K 31/438; A61K 31/444; A61K 31/497; A61K 31/501; A61K 31/506

(57) ABSTRACT

The disclosure provides at least one compound, tautomer, deuterated derivative, or pharmaceutically acceptable salt chosen from compounds of Formula I, tautomers thereof, deuterated derivatives of those compounds or tautomers, and pharmaceutically acceptable salts of any of the foregoing, compositions comprising the same, and methods of using the same, including uses in treating APOL1-mediated diseases, including pancreatic cancer, focal segmental glomerulosclerosis (FSGS), and/or non-diabetic kidney disease (NDKD).

This application claims the benefit of priority of U.S. Provisional Application No. 63/307,876, filed Feb. 8, 2022, the entire disclosure of which is incorporated herein by reference.

This disclosure provides compounds that may inhibit apolipoprotein L1 (APOL1) and methods of using those compounds to treat APOL1-mediated diseases, such as, e.g., pancreatic cancer, focal segmental glomerulosclerosis (FSGS), and/or non-diabetic kidney disease (NDKD). In some embodiments, the FSGS and/or NDKD is associated with at least one of the 2 common APOL1 genetic variants (G1: S342G:I384M and G2: N388del:Y389del). In some embodiments, the pancreatic cancer is associated with elevated levels of APOL1 (such as, e.g., elevated levels of APOL1 in pancreatic cancer tissues).

FSGS is a rare kidney disease with an estimated global incidence of 0.2 to 1.1/100,000/year. FSGS is a disease of the podocyte (glomerular visceral epithelial cells) responsible for proteinuria and progressive decline in kidney function. NDKD is a kidney disease involving damage to the podocyte or glomerular vascular bed that is not attributable to diabetes. NDKD is a disease characterized by hypertension and progressive decline in kidney function. Human genetics support a causal role for the G1 and G2 APOL1 variants in inducing kidney disease. Individuals with 2 APOL1 alleles are at increased risk of developing end-stage kidney disease (ESKD), including primary (idiopathic) FSGS, human immunodeficiency virus (HIV)_associated FSGS, NDKD, arterionephrosclerosis, lupus nephritis, microalbuminuria, and chronic kidney disease. See, P. Dummer et al., Semin Nephrol. 35(3): 222-236 (2015).

FSGS and NDKD can be divided into different subgroups based on the underlying etiology. One homogeneous subgroup of FSGS is characterized by the presence of independent common sequence variants in the apolipoprotein L1 (APOL1) gene termed G1 and G2, which are referred to as the “APOL1 risk alleles.” G1 encodes a correlated pair of non-synonymous amino acid changes (S342G and I384M), G2 encodes a 2 amino acid deletion (N388del:Y389del) near the C terminus of the protein, and G0 is the ancestral (low risk) allele. A distinct phenotype of NDKD is found in patients with APOL1 genetic risk variants as well. In both APOL1-mediated FSGS and NDKD, higher levels of proteinuria and a more accelerated loss of kidney function occur in patients with two risk alleles compared to patients with the same disease who have no or just 1 APOL1 genetic risk variant. Alternatively in AMKD, higher levels of proteinuria and accelerated loss of kidney function can also occur in patients with one risk allele. See, G. Vajgel et al., J. Rheumatol., November 2019, jrheum.190684.

APOL1 is a 44 kDa protein that is only expressed in humans, gorillas, and baboons. The APOL1 gene is expressed in multiple organs in humans, including the liver and kidney. APOL1 is produced mainly by the liver and contains a signal peptide that allows for secretion into the bloodstream, where it circulates bound to a subset of high-density lipoproteins. APOL1 is responsible for protection against the invasive parasite, Trypanosoma brucei brucei (T. b. brucei). APOL1 is endocytosed by T. b. brucei and transported to lysosomes, where it inserts into the lysosomal membrane and forms pores that lead to parasite swelling and death.

While the ability to lyse T. b. brucei is shared by all 3 APOL1 variants (G0, G1, and G2), APOL1 G1 and G2 variants confer additional protection against parasite species that have evolved a serum resistant associated-protein (SRA) which inhibits APOL1 G0; APOL1 G1 and G2 variants confer additional protection against trypanosoma species that cause sleeping sickness. G1 and G2 variants evade inhibition by SRA; G1 confers additional protection against T. b. gambiense (which causes West African sleeping sickness) while G2 confers additional protection against T. b. rhodesiense (which causes East African sleeping sickness).

In the kidney, APOL1 is expressed in podocytes, endothelial cells (including glomerular endothelial cells), and some tubular cells. Podocyte-specific expression of APOL1 G1 or G2 (but not G0) in transgenic mice induces structural and functional changes, including albuminuria, decreased kidney function, podocyte abnormalities, and glomerulosclerosis. Consistent with these data, G1 and G2 variants of APOL1 play a causative role in inducing FSGS and accelerating its progression in humans. Individuals with APOL1 risk alleles (i.e., homozygous or compound heterozygous for the APOL1 G1 or APOL1 G2 alleles) have increased risk of developing FSGS and they are at risk for rapid decline in kidney function if they develop FSGS. Thus, inhibition of APOL1 could have a positive impact in individuals who harbor APOL1 risk alleles.

Although normal plasma concentrations of APOL1 are relatively high and can vary at least 20-fold in humans, circulating APOL1 is not causally associated with kidney disease. However, APOL1 in the kidney is thought to be responsible for the development of kidney diseases, including FSGS and NDKD. Under certain circumstances, APOL1 protein synthesis can be increased by approximately 200-fold by pro-inflammatory cytokines such as interferons or tumor necrosis factor-α. In addition, several studies have shown that APOL1 protein can form pH-gated Na⁺/K⁺ pores in the cell membrane, resulting in a net efflux of intracellular K⁺, ultimately resulting in activation of local and systemic inflammatory responses, cell swelling, and death.

The risk of ESKD is substantially higher in people of recent sub-Saharan African ancestry as compared to those of European ancestry. In the United States, ESKD is responsible for nearly as many lost years of life in women as from breast cancer and more lost years of life in men than from colorectal cancer.

FSGS and NDKD are caused by damage to podocytes, which are part of the glomerular filtration barrier, resulting in proteinuria. Patients with proteinuria are at a higher risk of developing end-stage kidney disease (ESKD) and developing proteinuria-related complications, such as infections or thromboembolic events. There is no standardized treatment regimen nor approved drugs for FSGS or NDKD. Currently, FSGS and NDKD are managed with symptomatic treatment (including blood pressure control using blockers of the renin angiotensin system), and patients with FSGS and heavy proteinuria may be offered high dose steroids. Current therapeutic options for NDKD are anchored on blood pressure control and blockade of the renin angiotensin system.

Corticosteroids, alone or in combination with other immunosuppressants, induce remission in a minority of patients (e.g., remission of proteinuria in a minority of patients) and are associated with numerous side effects. However, remission is frequently indurable even in patients initially responsive to corticosteroid and/or immunosuppressant treatment. As a result, patients, in particular individuals of recent sub-Saharan African ancestry with 2 APOL1 risk alleles, experience rapid disease progression leading to end-stage renal disease (ESRD). Thus, there is an unmet medical need for treatment for FSGS and NDKD. Illustratively, in view of evidence that APOL1 plays a causative role in inducing and accelerating the progression of kidney disease, inhibition of APOL1 should have a positive impact on patients with APOL1 mediated kidney disease, particularly those who carry two APOL1 risk alleles (i.e., are homozygous or compound heterozygous for the G1 or G2 alleles).

Additionally, APOL1 is an aberrantly expressed gene in multiple cancers (Lin et al., Cell Death and Disease (2021), 12:760). Recently, APOL1 was found to be abnormally elevated in human pancreatic cancer tissues compared with adjacent tissues and was associated with poor prognosis in pancreatic cancer patients. In in vivo and in vitro experiments, knockdown of APOL1 significantly inhibited cancer cell proliferation and promoted the apoptosis of pancreatic cancer cells.

One aspect of the disclosure provides at least one compound, tautomer, deuterated derivative, or pharmaceutically acceptable salt chosen from compounds of Formula I, tautomers of Formula I, deuterated derivatives of those compounds or tautomers, and pharmaceutically acceptable salts of any of the foregoing, which can be employed in the treatment of diseases mediated by APOL1, such as FSGS and NDKD. For example, in some embodiments, the at least one compound is a compound represented by Formula I:

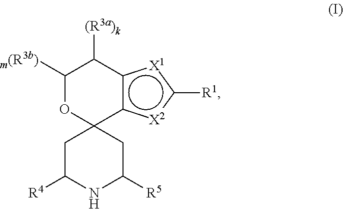

a tautomer thereof, a deuterated derivative of that compound or tautomer, or a pharmaceutically acceptable salt of any of the foregoing, wherein:
X¹ is chosen from S and —CR^{2a} and X² is chosen from S and —CR^{2b}, wherein:
   one of X¹ and X² is S;
   when X¹ is S, then X² is —CR^{2b}; and
   when X² is S, then X¹ is —CR^{2a};
R¹ is chosen from hydrogen, halogen, cyano, —OH, C₁-C₆ alkyl, C₁-C₆ alkoxy, C₃-C₆ cycloalkyl, 5- to 8-membered heterocyclyl, and phenyl, wherein:
   the C₁-C₆ alkyl of R¹ is optionally substituted with 1 to 3 groups independently chosen from halogen, cyano, 5- to 8-membered heterocyclyl (optionally substituted with 1 to 3 halogen groups), —OH, —NH₂, —NH(C₁-C₄ alkyl), —N(C₁-C₄ alkyl)₂, and C₁-C₄ alkoxy (optionally substituted with 1 to 3 halogen groups);
   the C₁-C₆ alkoxy of R¹ is optionally substituted with 1 to 3 groups independently chosen from halogen;
   the C₃-C₆ cycloalkyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂; and
   the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂;
R^{2a} is chosen from hydrogen, halogen, cyano, —OH, oxo, and C₁-C₆ alkyl, wherein:
   the C₁-C₆ alkyl of R^{2a} is optionally substituted with 1 to 3 groups independently chosen from halogen, cyano, —OH, and C₁-C₄ alkoxy;
R^{2b} is chosen from hydrogen, halogen, cyano, —OH, oxo, and C₁-C₆ alkyl;
each R^{3a} is independently chosen from halogen, cyano, —OH, C₁-C₆ alkoxy, and C₁-C₆ alkyl (optionally substituted with 1 to 3 groups independently chosen from halogen, cyano, and —OH); or
two R^{3a} together form an oxo group;
each R^{3b} is independently chosen from C₁-C₂ alkyl (optionally substituted with 1 to 3 groups independently chosen from halogen, cyano, and —OH); or
two R^{3b} together form an oxo group;
one of R⁴ and R⁵ is hydrogen and the other is chosen from C₁-C₆ alkyl, —C(═O)NH₂, —C(═O)O(C₁-C₄ alkyl), C₂-C₆ alkynyl, and

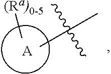

wherein:
   the C₁-C₆ alkyl of R⁴ or R⁵ optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ cycloalkyl, 5 to 10-membered heterocyclyl, phenyl, and 5 to 10-membered heteroaryl;
   Ring A is chosen from C₃-C₁₂ cycloalkyl, 3- to 12-membered heterocyclyl, C₆ and C₁₀ aryl, and 5- to 10-membered heteroaryl, wherein Ring A is optionally substituted with 1, 2, 3, 4, or 5 R^{a} groups, wherein:
   R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(═O)R^{k}, —NR^{h}(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ cycloalkyl, 3- to 12-membered heterocyclyl, C₆ and C₁₀ aryl, and 5- to 10-membered heteroaryl, wherein:
      the C₁-C₆ alkyl, the C₁-C₆ alkoxy, the C₁-C₆ haloalkyl, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5- to 10-membered heteroaryl (optionally substituted with 1 to 3 R^{m} groups), cyano,
   —C(═O)R^{k}, —C(═O)OR^{k}, —C(═O)NR^{h}Rⁱ, —NR^{h}Rⁱ, —NR^{h} C(═O)R^{k}, —NR^{h}C(═O)OR^{k},
   —NR^{h}C(═O)NRⁱR^{j}, —NR^{h} S(═O)ₚR^{k}, —OR^{k}, —OC(═O)R^{k}, —OC(═O)OR^{k}, —OC(═O)NR^{h}Rⁱ, —S(O)ₚR^{k}, —S(═O)ₚNR^{h}Rⁱ, and C₃-C₆ cycloalkyl (optionally substituted with 1 to 3 R^{m} groups);
      the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from halogen, cyano, C₁-C₄ alkyl, —C(═O)NR^{h}Rⁱ, —NR^{h}Rⁱ, —OR^{k}, and oxo, wherein:
      R^{h}, Rⁱ, and R^{j}, for each occurrence, are each independently chosen from hydrogen, C₁-C₄ alkyl, C₆-C₁₀ aryl, and C₃-C₆ cycloalkyl, wherein:
         the C₁-C₄ alkyl of any one of R^{h}, Rⁱ, and R^{j} is optionally substituted with 1 to 3 groups independently chosen from halogen, cyano, and —OH;
      R^{k}, for each occurrence, is independently chosen from hydrogen, C₁-C₄ alkyl, 5- to 10-membered heterocyclyl, and C₃-C₆ carbocycles, wherein:
         the C₁-C₄ alkyl of any one of R^{k} is optionally substituted with 1 to 3 groups independently chosen from halogen, cyano, and —OH;
      R^{m}, for each occurrence, is independently chosen from halogen, cyano, oxo, C₁-C₆ alkyl, C₁-C₆ alkoxy, —S(═O)ₚR^{k}, and —OR^{k}, wherein:
         the C₁-C₆ alkyl of R^{m} is optionally substituted with 1 to 3 groups independently chosen from halogen, cyano, and —OH;
k is an integer chosen from 0, 1, and 2, wherein, when R^{3a} is oxo, k is 1;
m is an integer chosen from 0, 1, and 2, wherein, when R^{3a} is oxo, m is 1;
p, for each occurrence, is an integer chosen from 1 and 2; and
q and r, for each occurrence, is an integer independently chosen from 1, 2, 3, and 4.

In some embodiments, at least one compound, tautomer, deuterated derivative, or pharmaceutically acceptable salt of the disclosure is a compound represented by the structural Formulae IIa, IIb, IIc, IId, IIIa, IIIb, IIIc, and IIId, as follows:

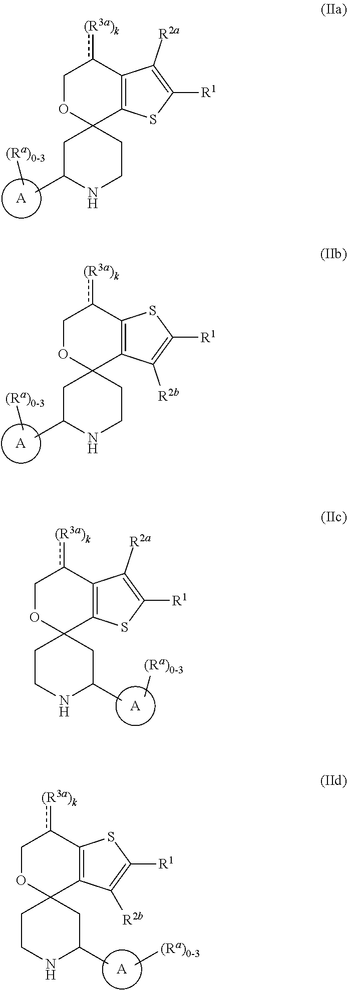

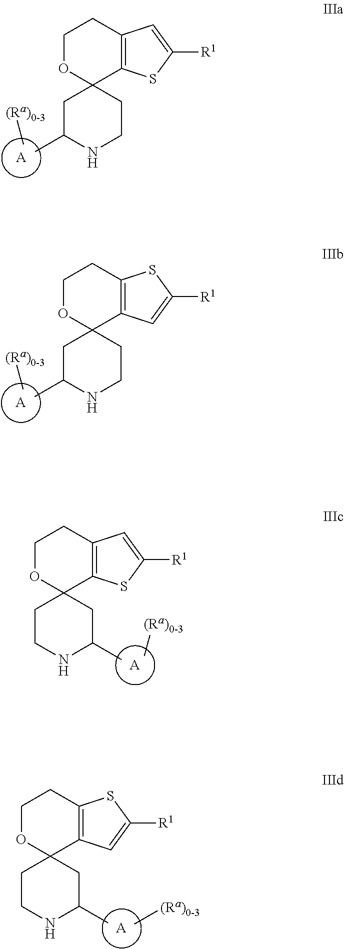

wherein Ring A, R^{a}, R¹, and R^{3a} are as defined above for Formula I.

In one aspect of the disclosure, the compounds of Formulae I, IIa, IIb, IIc, IId, IIIa, IIIb, IIIc, and IIId, are chosen from Compounds 1 to 78, tautomers thereof, deuterated derivatives of those compounds and tautomers and pharmaceutically acceptable salts of any of the foregoing.

In some embodiments, the disclosure provides a pharmaceutical composition comprising at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing. In some embodiments, the pharmaceutical composition may comprise at least one compound chosen from Compounds 1 to 78, tautomers thereof, deuterated derivatives of those compounds or tautomers, and pharmaceutically acceptable salts of any of the foregoing. These compositions may further include at least one additional active pharmaceutical ingredient and/or at least one carrier.

Another aspect of the disclosure provides methods of treating an APOL1-mediated disease comprising administering to a subject in need thereof, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing.

Another aspect of the disclosure provides methods of treating an APOL1-mediated cancer (such as, e.g., pancreatic cancer) comprising administering to a subject in need thereof, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing.

Another aspect of the disclosure provides methods of treating APOL1-mediated kidney disease (such as, e.g., ESKD, FSGS and/or NDKD) comprising administering to a subject in need thereof, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods comprise administering at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing.

In some embodiments, the methods of treatment include administration of at least one additional active agent to the subject in need thereof, either in the same pharmaceutical composition as the at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, or as separate compositions. In some embodiments, the methods comprise administering at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing with at least one additional active agent, either in the same pharmaceutical composition or in a separate composition.

Also provided are methods of inhibiting APOL1, comprising administering to a subject in need thereof, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt. In some embodiments, the methods of inhibiting APOL1 comprise administering at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing, or a pharmaceutical composition comprising the at least one compound, tautomer, deuterated derivative, or pharmaceutically acceptable salt.

### DETAILED DESCRIPTION

### Definitions

The term “APOL1,” as used herein, means apolipoprotein L1 protein and the term “APOL1” means apolipoprotein L1 gene.

The term “APOL1 mediated disease” refers to a disease or condition associated with aberrant APOL1 (e.g., certain APOL1 genetic variants; elevated levels of APOL1). In some embodiments, an APOL1 mediated disease is an APOL1 mediated kidney disease. In some embodiments, an APOL1 mediated disease is associated with patients having two APOL1 risk alleles, e.g., patients who are homozygous or compound heterozygous for the G1 or G2 alleles. In some embodiments, an APOL1 mediated disease is associated with patients having one APOL1 risk allele.

The term “APOL1 mediated kidney disease” refers to a disease or condition that impairs kidney function and can be attributed to APOL1. In some embodiments, APOL1 mediated kidney disease is associated with patients having two APOL1 risk alleles, e.g., patients who are homozygous or compound heterozygous for the G1 or G2 alleles. In some embodiments, the APOL1 mediated kidney disease is chosen from ESKD, NDKD, FSGS, HIV-associated nephropathy, arterionephrosclerosis, lupus nephritis, microalbuminuria, and chronic kidney disease. In some embodiments, the APOL1 mediated kidney disease is chronic kidney disease or proteinuria.

The term “FSGS,” as used herein, means focal segmental glomerulosclerosis, which is a disease of the podocyte (glomerular visceral epithelial cells) responsible for proteinuria and progressive decline in kidney function, and associated with 2 common APOL1 genetic variants (G1: S342G:1384M and G2: N388del:Y389del).

The term “NDKD,” as used herein, means non-diabetic kidney disease, which is characterized by severe hypertension and progressive decline in kidney function, and associated with 2 common APOL1 genetic variants (G1: S342G:I384M and G2: N388del:Y389del).

The terms “ESKD” and “ESRD” are used interchangeably herein to refer to end stage kidney disease or end stage renal disease. ESKD/ESRD is the last stage of kidney disease, i.e., kidney failure, and means that the kidneys have stopped working well enough for the patient to survive without dialysis or a kidney transplant. In some embodiments, ESKD/ESRD is associated with two APOL1 risk alleles.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms will also contain lesser amounts of isotopologues having hydrogen atoms at one or more of the designated deuterium positions in that structure. The relative amount of such isotopologues in a compound of this disclosure will depend upon a number of factors including the isotopic purity of reagents used to make the compound and the efficiency of incorporation of isotopes in the various synthesis steps used to prepare the compound. However, as set forth above, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, “optionally substituted” is interchangeable with the phrase “substituted or unsubstituted.” In general, the term “substituted,” whether preceded by the term “optionally” or not, refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position. Combinations of substituents envisioned by this disclosure are those that result in the formation of stable or chemically feasible compounds.

The term “isotopologue” refers to a species in which the chemical structure differs from a reference compound only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C or ¹⁴C, are within the scope of this disclosure.

Unless otherwise indicated, structures depicted herein are also meant to include all isomeric forms of the structures, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “tautomer,” as used herein, refers to one of two or more isomers of compound that exist together in equilibrium, and are readily interchanged by migration of an atom, e.g., a hydrogen atom, or group within the molecule.

“Stereoisomer,” as used herein, refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation, is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at well above its natural isotopic abundance (which is typically about 0.015%). In some embodiments, the deuterated derivatives of the disclosure have an isotopic enrichment factor for each deuterium atom, of at least 3500 (52.5% deuterium incorporation at each designated deuterium), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), or at least 6600 (99% deuterium incorporation).

The term “isotopic enrichment factor,” as used herein, means the ratio between the isotopic abundance and the natural abundance of a specified isotope.

The term “alkyl” or “aliphatic,” as used herein, means a straight-chain (i.e., linear or unbranched) or branched, substituted or unsubstituted hydrocarbon chain that is completely saturated. Unless otherwise specified, alkyl groups contain 1 to 20 alkyl carbon atoms. In some embodiments, alkyl groups contain 1 to 10 aliphatic carbon atoms. In some embodiments, alkyl groups contain 1 to 8 aliphatic carbon atoms. In some embodiments, alkyl groups contain 1 to 6 alkyl carbon atoms. In some embodiments, alkyl groups contain 1 to 4 alkyl carbon atoms, in other embodiments, alkyl groups contain 1 to 3 alkyl carbon atoms, and in yet other embodiments, alkyl groups contain 1 or 2 alkyl carbon atoms. In some embodiments, alkyl groups are linear or straight-chain or unbranched. In some embodiments, alkyl groups are branched.

The terms “cycloalkyl” and “cyclic alkyl,” as used herein, refer to a monocyclic C₃₋₈ hydrocarbon or a spirocyclic, fused, or bridged bicyclic or tricyclic C₈₋₁₄ hydrocarbon that is completely saturated, wherein any individual ring in said bicyclic ring system has 3 to 7 members. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The terms “cycloalkyl” or “cycloaliphatic,” as used herein, encompass the terms “cycloalkyl” or “cyclic alkyl,” and refer to a monocyclic C₃₋₈ hydrocarbon or a spirocyclic, fused, or bridged bicyclic or tricyclic C₈₋₁₄ hydrocarbon that is completely saturated, or is partially saturated as in it contains one or more units of unsaturation but is not aromatic, wherein any individual ring in said bicyclic ring system has 3 to 7 members. Bicyclic cycloalkyls include combinations of a monocyclic carbocyclic ring fused to a phenyl. In some embodiments, the cycloalkyl is a C₃ to C₁₂ cycloalkyl. In some embodiments, the cycloalkyl is a C₃ to C₁₀ cycloalkyl. In some embodiments, the cycloalkyl is a C₃ to C₈ cycloalkyl.

The term “heteroalkyl,” or “heteroaliphatic,” as used herein, means an alkyl or aliphatic group as defined above, wherein one or two carbon atoms are independently replaced by one or more of oxygen, sulfur, nitrogen, phosphorus, or silicon.

The term “alkenyl,” as used herein, means a straight-chain (i.e., linear or unbranched) or branched hydrocarbon chain that contains one or more double bonds. In some embodiments, alkenyl groups are straight-chain. In some embodiments, alkenyl groups are branched.

The terms “heterocycle,” “heterocyclyl,” and “heterocyclic,” are used herein interchangeably to refer to non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic cycloalkyl/cycloalkyl; and a monocyclic heteroaryl fused to a monocyclic cycloalkyl/cycloalkyl.

In some embodiments, the heterocycle comprises a ring atom substituted with one or more oxo groups (such as, e.g., a C═O group, a S═O group, or a SO₂ group).

In some embodiments, the “heterocycle,” “heterocyclyl,” “heterocycloaliphatic,” or “heterocyclic” group has 3 to 14 ring members in which one or more ring members is a heteroatom independently chosen from oxygen, sulfur, nitrogen, silicon, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, silicon, and phosphorus, the quaternized form of any basic nitrogen or; a substitutable nitrogen of a heterocyclic ring, for example, N (as in 3,4-dihydro-2H-pyrrolyl), NH (as in pyrrolidinyl) or NR⁺ (as in N-substituted pyrrolidinyl)). In some embodiments, the heterocycle has one heteroatom that is a nitrogen atom. In some embodiments, the heterocycle has one heteroatom that is an oxygen atom. In some embodiments, the heterocycle has two heteroatoms that are each independently chosen from nitrogen and oxygen. In some embodiments, the heterocycle has three heteroatoms that are each independently chosen from nitrogen and oxygen. In some embodiments, the heterocyclyl is a 3- to 12-membered heterocyclyl. In some embodiments, the heterocyclyl is a 3- to 10-membered heterocyclyl. In some embodiments, the heterocyclyl is a 3- to 8-membered heterocyclyl. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. Non-limiting examples of monocyclic heterocyclyls include piperidinyl, piperazinyl, tetrahydropyranyl, azetidinyl, tetrahydrothiophenyl 1,1-dioxide, and the like.

The term “unsaturated,” as used herein, means that a moiety has one or more units or degrees of unsaturation. Unsaturation is the state in which not all of the available valence bonds in a compound are satisfied by substituents and thus the compound contains double or triple bonds.

The term “alkoxy” or “thioalkyl,” as used herein, refers to an alkyl group, as previously defined, wherein one carbon of the alkyl group is replaced by an oxygen (“alkoxy”) or sulfur (“thioalkyl”) atom, respectively, provided that the oxygen and sulfur atoms are linked between two carbon atoms. A “cyclic alkoxy” refers to a monocyclic, spirocyclic, bicyclic, bridged bicyclic, tricyclic, or bridged tricyclic hydrocarbon that contains at least one alkoxy group, but is not aromatic. Non-limiting examples of cyclic alkoxy groups include tetrahydropyranyl, tetrahydrofuranyl, oxetanyl, 8-oxabicyclo[3.2.1]octanyl, and oxepanyl.

The terms “haloalkyl,” “haloalkenyl,” and “haloalkoxy,” as used herein, mean a linear or branched alkyl, alkenyl, or alkoxy, respectively, which is substituted with one or more halogen atoms. Non-limiting examples of haloalkyl groups include —CHF₂, —CH₂F, —CF₃, —CF₂—, and perhaloalkyls, such as —CF₂CF₃. Non-limiting examples of haloalkoxy groups include —OCHF₂, —OCH₂F, —OCF₃, and —OCF₂.

The term “halogen” includes F, Cl, Br, and I, i.e., fluoro, chloro, bromo, and iodo, respectively.

The term “aminoalkyl” means an alkyl group which is substituted with or contains an amino group.

As used herein, an “amino” refers to a group which is a primary, secondary, or tertiary amine.

As used herein, a “carbonyl” group refers to C═O.

As used herein, a “cyano” or “nitrile” group refer to —C≡N.

As used herein, a “hydroxy” group refers to —OH.

As used herein, a “thiol” group refers to —SH.

As used herein, “tert” and “t-” each refer to tertiary.

As used herein, “aromatic groups” or “aromatic rings” refer to chemical groups that contain conjugated, planar ring systems with delocalized pi electron orbitals comprised of [4n+2]p orbital electrons, wherein n is an integer ranging from 0 to 6. Non-limiting examples of aromatic groups include aryl and heteroaryl groups.

The term “aryl,” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Non-limiting examples of aryl groups include phenyl (C₆) and naphthyl (C₁₀) rings.

The term “heteroaryl,” used alone or as part of a larger moiety as in “heteroarylalkyl” or “heteroarylalkoxy,” refers to monocyclic or spirocyclic, fused, or bridged bicyclic or tricyclic ring systems having a total of five to fourteen ring members, wherein at least one ring in the system is aromatic, wherein at least one ring in the system contains one or more heteroatoms, and wherein each ring in a bicyclic or tricyclic ring system contains 3 to 7 ring members. Bicyclic heteroaryls include the following combinations of monocyclic rings: a monocyclic heteroaryl fused to another monocyclic heteroaryl; and a monocyclic heteroaryl fused to a phenyl. In some embodiments, heteroaryl groups have one or more heteroatoms chosen from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, the heteroaryl is a 3- to 12-membered heteroaryl. In some embodiments, the heteroaryl is a 3- to 10-membered heteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. Non-limiting examples of monocyclic heteroaryls are pyridinyl, pyrimidinyl, thiophenyl, thiazolyl, isoxazolyl, etc.

Non-limiting examples of useful protecting groups for nitrogen-containing groups, such as amine groups, include, for example, t-butyl carbamate (Boc), benzyl (Bn), tetrahydropyranyl (THP), 9-fluorenylmethyl carbamate (Fmoc) benzyl carbamate (Cbz), acetamide, trifluoroacetamide, triphenylmethylamine, benzylideneamine, and p-toluenesulfonamide. Methods of adding (a process generally referred to as “protecting”) and removing (process generally referred to as “deprotecting”) such amine protecting groups are well-known in the art and available, for example, in P. J. Kocienski, Protecting Groups, Thieme, 1994, which is hereby incorporated by reference in its entirety and in Greene and Wuts, Protective Groups in Organic Synthesis, 3rd Edition (John Wiley & Sons, New York, 1999) and 4^{th} Edition (John Wiley & Sons, New Jersey, 2014).

Non-limiting examples of suitable solvents that may be used in this disclosure include, but are not limited to, water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptane, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include, but are not limited to, 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

The disclosure includes pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, 1 to 19.

Acids commonly employed to form pharmaceutically acceptable salts include inorganic acids such as hydrogen bisulfide, hydrochloric acid, hydrobromic acid, hydroiodic acid, sulfuric acid, and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid, and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate, and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄ alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate, and aryl sulfonate. Other suitable, non-limiting examples of pharmaceutically acceptable salts include besylate and glucosamine salts.

The terms “patient” and “subject” are used interchangeably herein and refer to an animal, including a human.

The terms “effective dose” and “effective amount” are used interchangeably herein and refer to that amount of compound that produces a desired effect for which it is administered (e.g., improvement in a symptom of FSGS and/or NDKD, lessening the severity of FSGS and/NDKD or a symptom of FSGS and/or NDKD, and/or reducing progression of FSGS and/or NDKD or a symptom of FSGS and/or NDKD). The exact amount of an effective dose will depend on the purpose of the treatment and will be ascertainable by one skilled in the art using known techniques (see, e.g., Lloyd (1999) The Art, Science and Technology of Pharmaceutical Compounding).

As used herein, the term “treatment” and its cognates refer to slowing or stopping disease progression. “Treatment” and its cognates as used herein, include, but are not limited to, the following: complete or partial remission, lower risk of kidney failure (e.g., ESRD), and disease-related complications (e.g., edema, susceptibility to infections, or thrombo-embolic events). Improvements in or lessening the severity of any of these symptoms can be readily assessed according to methods and techniques known in the art or subsequently developed.

The terms “about” and “approximately,” when used in connection with doses, amounts, or weight percent of ingredients of a composition or a dosage form, include the value of a specified dose, amount, or weight percent or a range of the dose, amount, or weight percent that is recognized by one of ordinary skill in the art to provide a pharmacological effect equivalent to that obtained from the specified dose, amount, or weight percent.

The at least one compound, tautomer, deuterated derivative, or pharmaceutically acceptable salt chosen from compounds of Formulae I and II, tautomers thereof, deuterated derivatives of those compounds and tautomers, and pharmaceutically acceptable salts of any of the foregoing, may be administered once daily, twice daily, or three times daily, for example, for the treatment of AMKD, including FSGS and/or NDKD. In some embodiments, at least one compound chosen from Compounds 1 to 78, tautomers thereof, deuterated derivatives of those compounds and tautomers, and pharmaceutically acceptable salts of any of the foregoing may be administered once daily, twice daily, or three times daily, for example, for the treatment of AMKD, including FSGS and/or NDKD. In some embodiments, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds and tautomers, and pharmaceutically acceptable salts of any of the foregoing is administered once daily. In some embodiments, at least one compound chosed from Compounds 1 to 78, tautomers thereof, deuterated derivatives of those compounds and tautomers, and pharmaceutically acceptable salts of any of the foregoing is administered once daily. In some embodiments, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds and tautomers, and pharmaceutically acceptable salts of any of the foregoing is administered twice daily. In some embodiments, at least one compound chosed from Compounds 1 to 78, tautomers thereof, deuterated derivatives of those compounds and tautomers, and pharmaceutically acceptable salts of any of the foregoing is administered twice daily. In some embodiments, at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds and tautomers, and pharmaceutically acceptable salts of any of the foregoing is administered three times daily. In some embodiments, at least one compound chosed from Compounds 1 to 78, tautomers thereof, deuterated derivatives of those compounds and tautomers, and pharmaceutically acceptable salts of any of the foregoing is administered three times daily.

In some embodiments, 2 mg to 1500 mg or 5 mg to 1000 mg of at least one compound chosen from Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing is administered once daily, twice daily, or three times daily. In some embodiments, 2 mg to 1500 mg or 5 mg to 1000 mg of at least one compound chosen from Compounds 1 to 78, tautomera thereof, deuterated derivative of those compounds and tautomers, and pharmaceutically acceptable salts of any of the foregoing is administered once daily, twice daily, or three times daily.

One of ordinary skill in the art would recognize that, when an amount of compound is disclosed, the relevant amount of a pharmaceutically acceptable salt form of the compound is an amount equivalent to the concentration of the free base of the compound. The amounts of the compounds, pharmaceutically acceptable salts, solvates, and deuterated derivatives disclosed herein are based upon the free base form of the reference compound. For example, “1000 mg of at least one compound or pharmaceutically acceptable salt chosen from compounds of Formula I and pharmaceutically acceptable salts thereof” includes 1000 mg of a compound of Formula I and a concentration of a pharmaceutically acceptable salt of compounds of Formula I equivalent to 1000 mg of a compound of Formula I.

As used herein, the term “ambient conditions” means room temperature, open air condition, and uncontrolled humidity condition.

### Compounds and Compositions

In some embodiments, at least one compound chosen from Formulae I, IIa, IIb, IIc, IId, IIIa, IIIb, IIIc, and IIId, tautomers thereof, deuterated derivatives of those compounds and tautomers, and pharmaceutically acceptable salt of any of the foregoing may be employed in the treatment of AMKD, including FSGS and NDKD. In some embodiments, the compound of Formulae I, IIa, IIb, IIc, IId, IIIa, IIIb, IIIc, and IIId, may be chosen from Compounds 1 to 78, tautomers thereof, deuterated derivatives of those compounds and tautomers, and pharmaceutically acceptable salts of any of the foregoing. In some embodiments, a pharmaceutical composition comprising at least one compound chosen from Formulae I, IIa, IIb, IIc, IId, IIIa, IIIb, IIIc, and IIId, tautomers thereof, deuterated derivatives of those compounds and tautomers, and pharmaceutically acceptable salt of any of the foregoing, may be employed in the treatment of AMKD, including FSGS and NDKD. In some embodiments the pharmaceutical composition comprises at least one compound chosen from Compounds 1 to 78, tautomers thereof, deuterated derivatives of those compounds and tautomers, and pharmaceutically acceptable salt of any of the foregoing.

In some embodiments of Formula I.

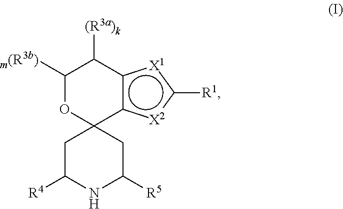

the variable X¹ is chosen from S and —CR^{2a} and X² is chosen from S and —CR^{2b}, wherein one of the variables X¹ and X² is S. In some embodiments of Formula I, the variable X¹ is S and the variable X² is —CR^{2b}. In some embodiments of Formula I, the variable X² is S and the variable X¹ is —CR^{2a}.

In some embodiments of Formula I (including the embodiments discussed above that define the variables X¹ and X²), the variable R¹ is chosen from hydrogen, halogen, cyano, —OH, C₁-C₆ alkyl, C₁-C₆ alkoxy, C₃-C₆ cycloalkyl, 5- to 8-membered heterocyclyl, and phenyl.

In some embodiments of Formula I (including the embodiments discussed above that define the variables X¹ and X²), the variable R¹ is chosen from halogen. In some embodiments of Formula I (including the embodiments discussed above that define the variables X¹ and X²), the variable R¹ is Cl. In some embodiments of Formula I (including the embodiments discussed above that define the variables X¹ and X²), the variable R¹ is Br. In some embodiments of Formula I (including the embodiments discussed above that define the variables X¹ and X²), the variable R¹ is I.

In some embodiments of Formula I (including the embodiments discussed above that define the variables X¹ and X²), the variable R¹ is chosen from C₁-C₆ alkyl. In some embodiments of Formula I (including the embodiments discussed above that define the variables X¹ and X²), the variable R¹ is C₁ alkyl. In some embodiments of Formula I (including the embodiments discussed above that define the variables X¹ and X²), the variable R¹ is C₂ alkyl.

In some embodiments of Formula I (including the embodiments discussed above that define the variables X¹ and X²), the C₁-C₆ alkyl of R¹ is optionally substituted with 1 to 3 groups independently chosen from halogen, cyano, 5- to 8-membered heterocyclyl (optionally substituted with 1 to 3 halogen groups), —OH, —NH₂, —NH(C₁-C₄ alkyl), —N(C₁-C₄ alkyl)₂, and C₁-C₄ alkoxy (optionally substituted with 1 to 3 halogen groups). In some embodiments of Formula I (including the embodiments discussed above that define the variables X¹ and X²), the C₁-C₆ alkyl of R¹ is optionally substituted with 1 to 3 groups independently chosen from halogen. In some embodiments of Formula I (including the embodiments discussed above that define the variables X¹ and X²), the C₁-C₆ alkyl of R¹ is substituted with 1 halogen. In some embodiments of Formula I (including the embodiments discussed above that define the variables X¹ and X²), the C₁-C₆ alkyl of R¹ is substituted with 2 halogen. In some embodiments of Formula I (including the embodiments discussed above that define the variables X¹ and X²), the C₁-C₆ alkyl of R¹ is substituted with 3 halogen. In some embodiments of Formula I (including the embodiments discussed above that define the variables X¹ and X²), the C₁-C₆ alkyl of R¹ is substituted with 1 F. In some embodiments of Formula I (including the embodiments discussed above that define the variables X¹ and X²), the C₁-C₆ alkyl of R¹ is substituted with 2 F. In some embodiments of Formula I (including the embodiments discussed above that define the variables X¹ and X²), the C₁-C₆ alkyl of R¹ is substituted with 3 F. In some embodiments of Formula I (including the embodiments discussed above that define the variables X¹ and X²), R¹ is —CF₃. In some embodiments of Formula I (including the embodiments discussed above that define the variables X¹ and X²), R¹ is —CH₂CHF₂. In some embodiments of Formula I (including the embodiments discussed above that define the variables X¹ and X²), R¹ is —CH₂CF₃.

In some embodiments of Formula I (including the embodiments discussed above that define the variables X¹ and X²), the variable R¹ is chosen from C₁-C₆ alkoxy. In some embodiments of Formula I (including the embodiments discussed above that define the variables X¹ and X²), the C₁-C₆ alkoxy of R¹ is optionally substituted with 1 to 3 groups independently chosen from halogen.

In some embodiments of Formula I (including the embodiments discussed above that define the variables X¹ and X²), the variable R¹ is chosen from C₃-C₆ cycloalkyl. In some embodiments of Formula I (including the embodiments discussed above that define the variables X¹ and X²), the variable R¹ is C₃ cycloalkyl. In some embodiments of Formula I (including the embodiments discussed above that define the variables X¹ and X²), the C₃-C₆ cycloalkyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂. In some embodiments of Formula I (including the embodiments discussed above that define the variables X¹ and X²), the C₃-C₆ cycloalkyl of R¹ is optionally substituted with 1 to 3 groups independently chosen from halogen. In some embodiments of Formula I (including the embodiments discussed above that define the variables X¹ and X²), the C₃-C₆ cycloalkyl of R¹ is substituted with 1 halogen. In some embodiments of Formula I (including the embodiments discussed above that define the variables X¹ and X²), the C₃-C₆ cycloalkyl of R¹ is substituted with 2 halogen. In some embodiments of Formula I (including the embodiments discussed above that define the variables X¹ and X²), the C₃-C₆ cycloalkyl of R¹ is substituted with 3 halogen. In some embodiments of Formula I (including the embodiments discussed above that define the variables X¹ and X²), R¹ is C₄ cycloalkyl substituted with 2 F. In some embodiments of Formula I (including the embodiments discussed above that define the variables X¹ and X²), R¹ is

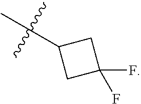 [SMILES: *C1CC(F)(F)C1]

In some embodiments of Formula I (including the embodiments discussed above that define the variables X¹ and X²), R¹ is chosen from phenyl. In some embodiments of Formula I (including the embodiments discussed above that define the variables X¹ and X²), the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂.

In some embodiments of Formula I (including the embodiments discussed above that define the variables X¹, X², and R¹), the variable R^{2a} is chosen from hydrogen, halogen, cyano, —OH, oxo, and C₁-C₆ alkyl, wherein C₁-C₆ alkyl of R^{2a} is optionally substituted with 1 to 3 groups independently chosen from halogen, cyano, —OH, and C₁-C₄ alkoxy. In some embodiments of Formula I (including the embodiments discussed above that define the variables X¹, X², and R¹), the variable R^{2a} is hydrogen. In some embodiments of Formula I (including the embodiments discussed above that define the variables X¹, X², and R¹), the variable R^{2a} is chosen from C₁-C₆ alkyl. In some embodiments of Formula I (including the embodiments discussed above that define the variables X¹, X², and R¹), the C₁-C₆ alkyl of R^{2a} is optionally substituted with 1 to 3 groups independently chosen from halogen, cyano, —OH, and C₁-C₄ alkoxy. In some embodiments of Formula I (including the embodiments discussed above that define the variables X¹, X², and R¹), the C₁-C₆ alkyl of R^{2a} is substituted with 1 to 3 —OH. In some embodiments of Formula I (including the embodiments discussed above that define the variables X¹, X², and R¹), the variable R^{2a} is —CH₂OH. In some embodiments of Formula I (including the embodiments discussed above that define the variables X¹, X², and R¹), the variable R^{2a} is —CHOHCH₃.

In some embodiments of Formula I (including the embodiments discussed above that define the variables X¹, X², R¹, and R^{2a}), the variable R^{2b} is chosen from hydrogen, halogen, cyano, —OH, oxo, and C₁-C₆ alkyl. In some embodiments of Formula I (including the embodiments discussed above that define the variables X¹, X², R¹, and R^{2a}), the variable R^{2b} is hydrogen.

In some embodiments of Formula I (including the embodiments discussed above that define the variables X¹, X², R¹, R^{2a}, and R^{2b}), each variable R^{3a} is independently chosen from halogen, cyano, —OH, C₁-C₆ alkyl (optionally substituted with 1 to 3 groups independently chosen from halogen, cyano, and —OH), C₁-C₆ alkoxy, and oxo.

In some embodiments of Formula I (including the embodiments discussed above that define the variables X¹, X², R¹, R^{2a}, and R^{2b}), the variable R^{3a} is —OH.

In some embodiments of Formula I (including the embodiments discussed above that define the variables X¹, X², R¹, R^{2a}, and R^{2b}), when two variables R^{3a} form an oxo, then R^{3a} is not oxo. In some embodiments of Formula I (including the embodiments discussed above that define the variables X¹, X², R¹, R^{2a}, and R^{2b}), when two variables R^{3a} form an oxo, then R^{3a} is not oxo.

In some embodiments of Formula I (including the embodiments discussed above that define the variables X¹, X², R¹, R^{2a}, and R^{2b}), each variable R^{3a} is independently chosen from C₁-C₆ alkyl. In some embodiments of Formula I (including the embodiments discussed above that define the variables X¹, X², R¹, R^{2a}, and R^{2b}), the variable R^{3a} is C₁ alkyl. In some embodiments of Formula I (including the embodiments discussed above that define the variables X¹, X², R¹, R^{2a}, and R^{2b}), the variable R^{3a} is —CH₃. In some embodiments of Formula I (including the embodiments discussed above that define the variables X¹, X², R¹, R^{2a}, and R^{2b}), the C₁-C₆ alkyl of R^{3a} is optionally substituted with 1 to 3 groups independently chosen from halogen, cyano, and —OH. In some embodiments of Formula I (including the embodiments discussed above that define the variables X¹, X², R¹, R^{2a}, and R^{2b}), the C₁-C₆ alkyl of R^{3a} is optionally substituted with 1 to 3 groups independently chosen from halogen. In some embodiments of Formula I (including the embodiments discussed above that define the variables X¹, X², R¹, R^{2a}, and R^{2b}), the variable R^{3a} is —CHCF₂.

In some embodiments of Formula I (including the embodiments discussed above that define the variables X¹, X², R¹, R^{2a}, and R^{2b}), each variable R^{3a} is independently chosen from C₁-C₆ alkoxy. In some embodiments of Formula I (including the embodiments discussed above that define the variables X¹, X², R¹, R^{2a}, and R^{2b}), the variable R^{3a} is —OCH₃.

In some embodiments of Formula I (including the embodiments discussed above that define the variables X¹, X², R¹, R^{2a}, and R^{2b}), the variable R^{3a} is oxo.

In some embodiments of Formula I (including the embodiments discussed above that define the variables X¹, X², R¹, R^{2a}, R^{2b}, and R^{3a}), the variable R^{3a} is chosen from C₁-C₂ alkyl and oxo. In some embodiments of Formula I (including the embodiments discussed above that define the variables X¹, X², R¹, R^{2a}, R^{2b}, and R^{3a}), the C₁-C₂ alkyl of R³ is optionally substituted with 1 to 3 groups independently chosen from halogen, cyano, and —OH.

In some embodiments of Formula I (including the embodiments discussed above that define the variables X¹, X², R¹, R^{2a}, R^{2b}, R^{3a}, and R^{3b}), one of R⁴ and R⁵ is hydrogen and the other is chosen from C₁-C₆ alkyl, —C(═O)NH₂, —C(═O)O(C₁-C₄ alkyl), C₂-C₆ alkynyl, and

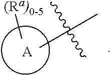

In some embodiments of Formula I (including the embodiments discussed above that define the variables X¹, X², R¹, R^{2a}, R^{2b}, R^{3a}, and R^{3b}), the C₁-C₆ alkyl of R⁴ or R⁵ optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ cycloalkyl, 5 to 10-membered heterocyclyl, phenyl, and 5 to 10-membered heteroaryl.

In some embodiments of Formula I (including the embodiments discussed above that define the variables X¹, X², R¹, R^{2a}, R^{2b}, R^{3a}, and R^{3b}), the variable R⁴ is hydrogen and the variable R⁵ is chosen from

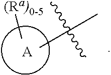

In some embodiments of Formula I (including the embodiments discussed above that define the variables X¹, X², R¹, R^{2a}, R^{2b}, R^{3a}, and R^{3b}), the variable R⁴ is chosen from

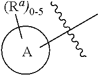

and the variable R⁵ is hydrogen.

In some embodiments of Formula I (including the embodiments discussed above that define the variables X¹, X², R¹, R^{2a}, R^{2b}, R^{3a}, R^{3b}, R⁴, and R⁵), the variable Ring A is chosen from C₃-C₁₂ cycloalkyl, 3- to 12-membered heterocyclyl, C₆ and C₁₀ aryl, and 5- to 10-membered heteroaryl. In some embodiments of Formula I (including the embodiments discussed above that define the variables X¹, X², R¹, R^{2a}, R^{2b}, R^{3a}, R^{3b}, R⁴, and R⁵), the variable Ring A is (optionally substituted with 1, 2, 3, 4, or 5 R^{a} groups).

In some embodiments of Formula I (including the embodiments discussed above that define the variables X¹, X², R¹, R^{2a}, R^{2b}, R^{3a}, R^{3b}, R⁴, and R⁵), the variable Ring A is chosen from C₃-C₁₂ cycloalkyl (optionally substituted with 1, 2, 3, 4, or 5 R^{a} groups). In some embodiments of Formula I (including the embodiments discussed above that define the variables X¹, X², R¹, R^{2a}, R^{2b}, R^{3a}, R^{3b}, R⁴, and R⁵), the variable Ring A is chosen from C₃ cycloalkyl (optionally substituted with 1, 2, 3, 4, or 5 R^{a} groups). In some embodiments of Formula I (including the embodiments discussed above that define the variables X¹, X², R¹, R^{2a}, R^{2b}, R^{3a}, R^{3b}, R⁴, and R⁵), the variable Ring A is chosen from C₄ cycloalkyl (optionally substituted with 1, 2, 3, 4, or 5 R^{a} groups). In some embodiments of Formula I (including the embodiments discussed above that define the variables X¹, X², R¹, R^{2a}, R^{2b}, R^{3a}, R^{3b}, R⁴, R⁵, and Ring A), the variable Ring A chosen from

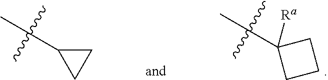

In some embodiments of Formula I (including the embodiments discussed above that define the variables X¹, X², R¹, R^{2a}, R^{2b}, R^{3a}, R^{3b}, R⁴, and R⁵), the variable Ring A is chosen from C₆ aryl (optionally substituted with 1, 2, 3, 4, or 5 R^{a} groups). In some embodiments of Formula I (including the embodiments discussed above that define the variables X¹, X², R¹, R^{2a}, R^{2b}, R^{3a}, R^{3b}, R⁴, and R⁵), the variable Ring A is chosen from

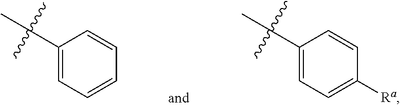

In some embodiments of Formula I (including the embodiments discussed above that define the variables X¹, X², R¹, R^{2a}, R^{2b}, R^{3a}, R^{3b}, R⁴, and R⁵), the variable Ring A is chosen from 5- to 10-membered heteroaryl (optionally substituted with 1, 2, 3, 4, or 5 R^{a} groups). In some embodiments of Formula I (including the embodiments discussed above that define the variables X¹, X², R¹, R^{2a}, R^{2b}, R^{3a}, R^{3b}, R⁴, and R⁵), the variable Ring A is chosen from 5-membered heteroaryl (optionally substituted with 1, 2, 3, 4, or 5 R^{a}groups). In some embodiments of Formula I (including the embodiments discussed above that define the variables X¹, X², R¹, R^{2a}, R^{2b}, R^{3a}, R^{3b}, R⁴, and R⁵), the variable Ring A is chosen from 6-membered heteroaryl (optionally substituted with 1, 2, 3, 4, or 5 R^{a}groups). In some embodiments of Formula I (including the embodiments discussed above that define the variables X¹, X², R¹, R^{2a}, R^{2b}, R^{3a}, R^{3b}, R⁴, and R⁵), the variable Ring A is chosen from:

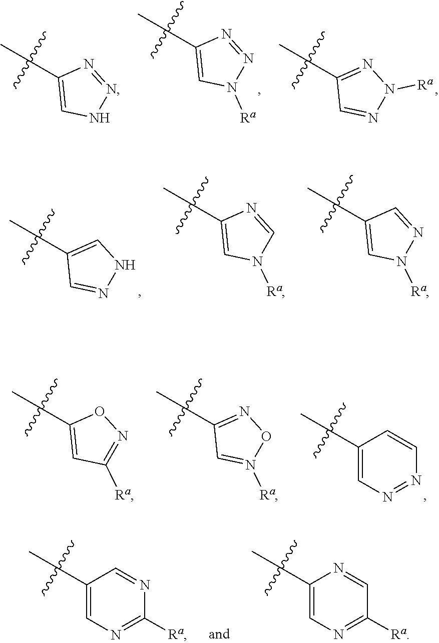

In some embodiments of Formula I (including the embodiments discussed above that define the variables X¹, X², R¹, R^{2a}, R^{2b}, R^{3a}, R^{3b}, R⁴, R⁵, and Ring A), the variable R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(═O)R^{k}, —NR^{h} C(O)OR^{k}, —NR^{h}C(═O)NRⁱRⁱ, —NR^{h}S(═O)ₚR^{k}, —OR^{k}, —OC(═O)R^{k}, —OC(═O)OR^{k}, —OC(═O)NR^{h}Rⁱ, —[O(CH2)_{q}]ᵣO(C₁-C₆ alkyl), —S(═O)ₚR^{k}, —S(═O)ₚNR^{h}Rⁱ, —C(═O)OR^{k}, C₃-C₁₂ cycloalkyl, 3- to 12-membered heterocyclyl, C₆ and C₁₀ aryl, and 5- to 10-membered heteroaryl.

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from halogen. In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is F.

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from C₁-C₆ alkyl. In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is C₁ alkyl. In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is —CH₃. In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is C₂ alkyl.

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from —C(═O)NR^{h}Rⁱ. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ, for each occurrence, are each independently chosen from hydrogen and C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ are each hydrogen. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ are independently selected from C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), one of the variables R^{h} and Rⁱ is hydrogen and the other is C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), one of the variables R^{h} and Rⁱ is hydrogen and the other is —CH₃. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ are each —CH₃.

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from —OR^{k}. In some embodiments of Formula I (including the embodiments discussed above that define the variables X¹, X², R¹, R^{2a}, R^{2b}, R^{3a}, R^{3b}, R⁴, R⁵, and Ring A), the variable R¹, for each occurrence, is independently chosen from hydrogen, C₁-C₄ alkyl, 5- to 10-membered heterocyclyl, and C₃-C₆ carbocycles, wherein the C₁-C₄ alkyl of any one of R¹ is optionally substituted with 1 to 3 groups independently chosen from halogen, cyano, and —OH. In some embodiments of Formula I (including the embodiments discussed above that define the variables X¹, X², R¹, R^{2a}, R^{2b}, R^{3a}, R^{3b}, R⁴, R⁵, and Ring A), the variable R^{k} is hydrogen. In some embodiments of Formula I (including the embodiments discussed above that define the variables X¹, X², R¹, R^{2a}, R^{2b}, R^{3a}, R^{3b}, R⁴, R⁵, and Ring A), the variable R^{k} is —CH₃.

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from 3- to 12-membered heterocyclyl. In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is

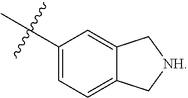 [SMILES: *c1ccc2c(c1)CNC2]

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from C₆ aryl.

In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen from 5- to 10-membered heteroaryl. In some embodiments of Formula I (including the embodiments discussed above that define the variables X¹, X², R¹, R^{2a}, R^{2b}, R^{3a}, R^{3b}, R⁴, R⁵, and Ring A), the variable R^{a} is chosen fro

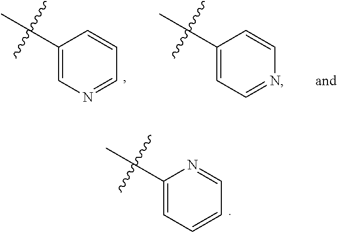 [SMILES: *c1cccnc1 ; "," ; *c1ccncc1 ; "and" ; *c1ccccn1 ; "."]

In some embodiments of Formula I (including the embodiments discussed above that define the variables X¹, X², R¹, R^{2a}, R^{2b}, R^{3a}, R^{3b}, R⁴, R⁵, and Ring A), the C₁-C₆ alkyl, the C₁-C₆ alkoxy, the C₁-C₆ haloalkyl, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5- to 10-membered heteroaryl (optionally substituted with 1 to 3 R^{m} groups), cyano, —C(═O)R^{k}, —C(═O)OR^{k}, —C(═O)NR^{h}Rⁱ, —NR^{h}Rⁱ, —NR^{h}C(═O)R^{k}, —NR^{h}(═O)OR^{k}, —NR^{h}C(═O)NRⁱR^{j}, —NR^{h}S(═O)ₚR^{k}, —OR^{k}, —OC(═O)R^{k}, —OC(═O)OR^{k}, —OC(═O)NR^{h}Rⁱ, —S(═O)ₚR^{k}, —S(═O)ₚNR^{h}Rⁱ, and C₃-C₆ cycloalkyl (optionally substituted with 1 to 3 R^{m} groups).

In some embodiments of Formula I (including the embodiments discussed above that define the variables X¹, X², R¹, R^{2a}, R^{2b}, R^{3a}, R^{3b}, R⁴, R⁵, and Ring A), the C₁-C₆ alkyl, the C₁-C₆ alkoxy, the C₁-C₆ haloalkyl, and the C₂-C₆ alkenyl of R^{a} are each optionally substituted with 1 to 3 groups independently chosen from —OR^{k}. In some embodiments of Formula I (including the embodiments discussed above that define the variables X¹, X², R¹, R^{2a}, R^{2b}, R^{3a}, R^{3b}, R⁴, R⁵, and Ring A), the variable R^{k} is hydrogen. In some embodiments of Formula I (including the embodiments discussed above that define the variables X¹, X², R¹, R^{2a}, R^{2b}, R^{3a}, R^{3b}, R⁴, R⁵, and Ring A), the variable R^{k} is —CH₃.

In some embodiments of Formula I (including the embodiments discussed above that define the variables X¹, X², R¹, R^{2a}, R^{2b}, R^{3a}, R^{3b}, R⁴, R⁵, and Ring A), the C₁-C₆ alkyl, the C₁-C₆ alkoxy, the C₁-C₆ haloalkyl, and the C₂-C₆ alkenyl of R^{a} are each optionally substituted with 1 to 3 groups independently chosen from —S(═O),R^{k}. In some embodiments of Formula I (including the embodiments discussed above that define the variables X¹, X², R¹, R^{2a}, R^{2b}, R^{3a}, R^{3b}, R⁴, R⁵, and Ring A), the variable p is 2 and the variable R^{k} is —CH₃.

In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from halogen, cyano, C₁-C₄ alkyl, —C(═O)NR^{h}Rⁱ, —NR^{h}Rⁱ, —OR^{k}, and oxo.

In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from halogen. In some embodiments of Formula I (including the embodiments discussed above that define the variables X¹, X², R¹, R^{2a}, R^{2b}, R^{3a}, R^{A}b, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 F.

In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 —CH₃ groups.

In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from —C(═O)NR^{h}Rⁱ. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ, for each occurrence, are each independently chosen from hydrogen and C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ are each hydrogen. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rⁱ are independently selected from C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), one of the variables R^{h} and Rⁱ is hydrogen and the other is C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), one of the variables R^{h} and Rⁱ is hydrogen and the other is —CH₃. In some embodiments of Formula I (including the embodiments discussed above that define the variables X¹, X², R¹, R^{2a}, R^{2b}, R^{3a}, R^{3b}, R⁴, R⁵, and Ring A), the variables R^{h} and Rare each —CH₃.

In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from —OR^{k}. In some embodiments of Formula I (including the embodiments discussed above that define the variables X¹, X², R¹, R^{2a}, R^{2b}, R^{3a}, R^{3b}, R⁴, R⁵, and Ring A), the variable R¹ is hydrogen. In some embodiments of Formula I (including the embodiments discussed above that define the variables X¹, X², R¹, R^{2a}, R^{2b}, R^{3a}, R^{3b}, R⁴, R⁵, and Ring A), the variable R^{k} is —CH₃.

In some embodiments of Formula I (including the embodiments discussed above that define the variables X¹, X², R¹, R^{2a}, R^{2b}, R^{3a}, R^{3b}, R⁴, R⁵, and Ring A), the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 oxo.

In some embodiments of Formula I (including the embodiments discussed above that define the variables X¹, X², R¹, R^{2a}, R^{2b}, R^{3a}, R^{3b}, R⁴, R⁵, and Ring A), the variables R^{h}, Rⁱ, and R^{j}, for each occurrence, are each independently chosen from hydrogen, C₁-C₄ alkyl, C₆-C₁₀ aryl, and C₃-C₆ cycloalkyl. In some embodiments of Formula I (including the embodiments discussed above that define the variables X¹, X², R¹, R^{2a}, R^{2b}, R^{3a}, R^{3b}, R⁴, R⁵, and Ring A), the variables R^{h}, Rⁱ, and R^{j}, for each occurrence, are each independently chosen from hydrogen and C₁-C₄ alkyl. In some embodiments of Formula I (including the embodiments discussed above that define the variables X¹, X², R¹, R^{2a}, R^{2b}, R^{3a}, R^{3b}, R⁴, R⁵, and Ring A), the C₁-C₄ alkyl of any one of R^{h}, Rⁱ, and R^{j} is optionally substituted with 1 to 3 groups independently chosen from halogen, cyano, and —OH.

In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, and R^{j}), the variable R^{k}, for each occurrence, is independently chosen from hydrogen, C₁-C₄ alkyl, 5- to 10-membered heterocyclyl, and C₃-C₆ cycloalkyl. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, and R^{j}), the variable R^{k}, for each occurrence, is hydrogen. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, and R^{j}), the variable R^{k}, for each occurrence, is independently chosen from C₁-C₄ alkyl. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, and R^{j}), the C₁-C₄ alkyl of any one of R^{k} is optionally substituted with 1 to 3 groups independently chosen from halogen, cyano, and —OH.

In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, and R^{k}), the variable R¹, for each occurrence, is independently chosen from halogen, cyano, oxo, C₁-C₆ alkyl, C₁-C₆ alkoxy, —S(═O)ₚR^{k}, and OR^{k}. In some embodiments, C₁-C₆ alkyl of R^{m} is optionally substituted with 1 to 3 groups independently chosen from halogen, cyano, and —OH.

In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, and R^{m}), the variable k is an integer chosen from 0, 1, and 2. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, and R^{m}), when R^{3a} is oxo, k is 1.

In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, R^{m}, and k), the variable m is an integer chosen from 0, 1, and 2. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, R^{m}, and k), the variable m is 0. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, R^{m}, and k), when R³ is oxo, m is 1.

In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, R^{m}, and m), the variable p, for each occurrence, is an integer chosen from 1 and 2. In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, R^{m}, and m), the variable p is 2.

In some embodiments of Formula I (including the embodiments discussed above that define the variables X¹, X², R¹, R^{2a}, R^{2b}, R^{3a}, R^{3b}, R⁴, R⁵, Ring A, R^{h}, Rⁱ, R^{j}, R^{k}, R^{m}, m, and p), the variables q and r, for each occurrence, is an integer independently chosen from 1, 2, 3, and 4.

In some embodiments, the at least one compound, tautomer, deuterated derivative, or pharmaceutically acceptable salt of the disclosure is chosen from Compounds 1 to 78 depicted in Table 1, a tautomer thereof, a deuterated derivative of that compound or tautomer, or a pharmaceutically acceptable salt of any of the foregoing. A wavy line in a compound in Table 1 (i.e., [symbol] ) depicts a bond between two atoms and indicates a position of mixed stereochemistry for a collection of molecules, such as a racemic mixture, cis/trans isomers, or (E)/(Z) isomers. An asterisk adjacent to an atom

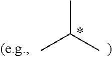

in a compound in Table 1, indicates a chiral position in the molecule.

In some embodiments, the compound of Formula I is selected from the compounds presented in Table 1 below, tautomers of those compounds, deuterated derivatives of those compounds and tautomers, and pharmaceutically acceptable salts of any of the foregoing.

TABLE 1
Compounds 1 to 78

| | |
|---|---|
| 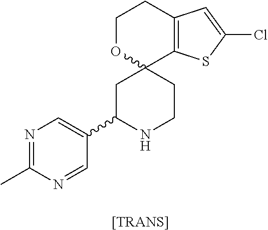 | 1 |
| 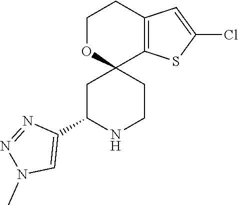 [SMILES: Cn1cc([C@@H]2C[C@]3(CCN2)OCCc2cc(Cl)sc23)nn1] | 2 |
| 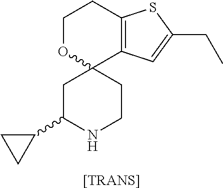 | 3 |
| 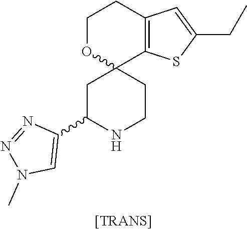 | 4 |
| 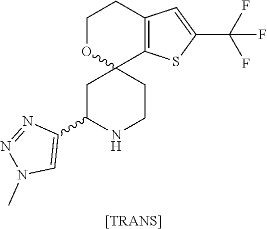 | 5 |
| 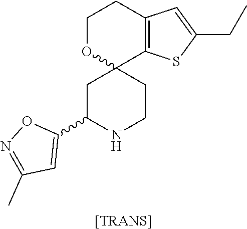 | 6 |
| 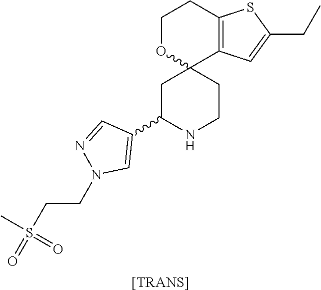 | 7 |
| 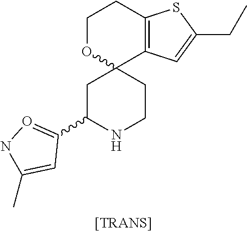 | 8 |
| 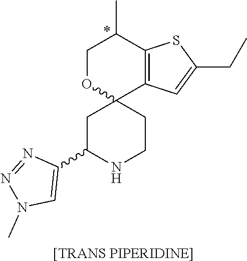 | 9 |
| 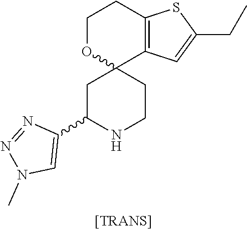 | 10 |
| 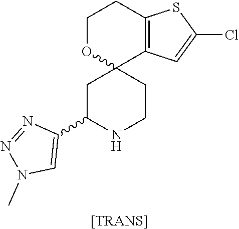 | 11 |
| 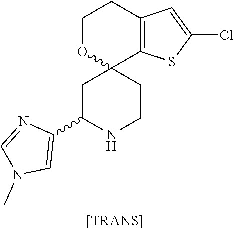 | 12 |
| 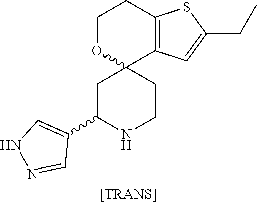 | 13 |
| 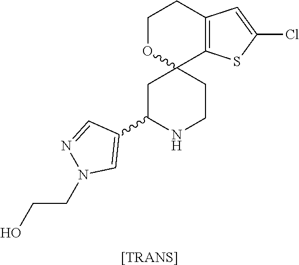 | 14 |
| 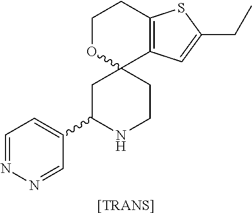 | 15 |
| 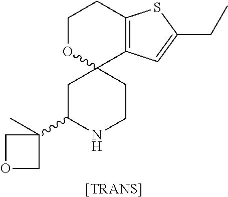 | 16 |
| 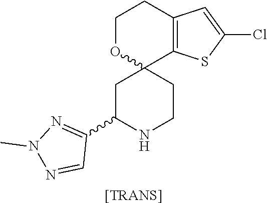 | 17 |
| 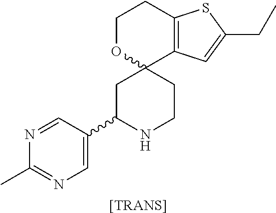 | 18 |
| 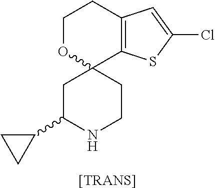 | 19 |
| 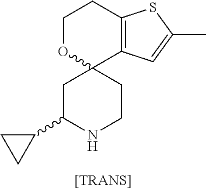 | 20 |
| 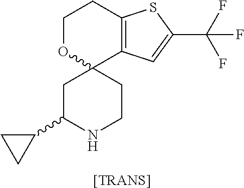 | 21 |
| 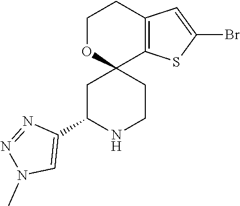 [SMILES: Cn1cc([C@@H]2C[C@]3(CCN2)OCCc2cc(Br)sc23)nn1] | 22 |
| 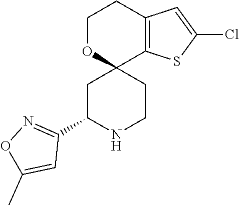 [SMILES: Cc1cc([C@@H]2C[C@]3(CCN2)OCCc2cc(Cl)sc23)no1] | 23 |
| 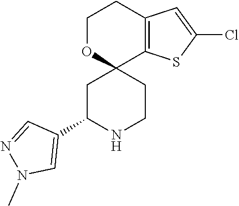 [SMILES: Cn1cc([C@@H]2C[C@]3(CCN2)OCCc2cc(Cl)sc23)cn1] | 24 |
| 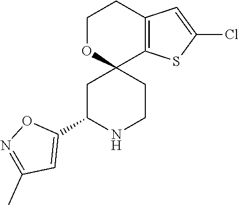 [SMILES: Cc1cc([C@@H]2C[C@]3(CCN2)OCCc2cc(Cl)sc23)on1] | 25 |
| 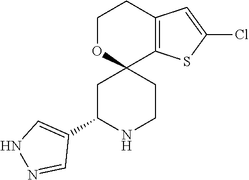 [SMILES: Clc1cc2c(s1)[C@@]1(CCN[C@H](c3cn[nH]c3)C1)OCC2] | 26 |
| 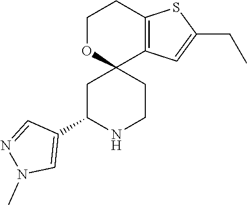 [SMILES: CCc1cc2c(s1)CCO[C@]21CCN[C@H](c2cnn(C)c2)C1] | 27 |
| 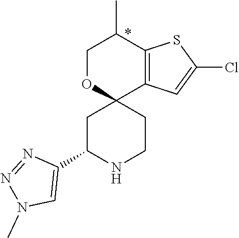 | 28 |
| 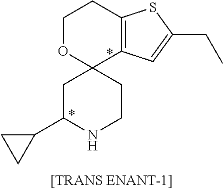 | 29 |
| 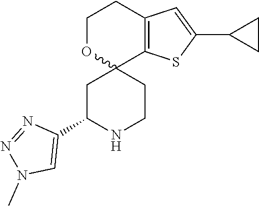 [SMILES: Cn1cc([C@@H]2CC3(CCN2)OCCc2cc(C4CC4)sc23)nn1] | 30 |
| 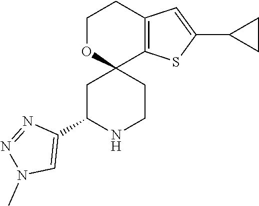 [SMILES: Cn1cc([C@@H]2C[C@]3(CCN2)OCCc2cc(C4CC4)sc23)nn1] | 31 |
| 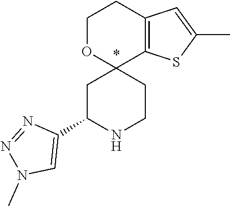 [SMILES: Cc1cc2c(s1)C1(CCN[C@H](c3cn(C)nn3)C1)OCC2] | 32 |
| 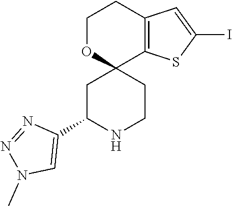 [SMILES: Cn1cc([C@@H]2C[C@]3(CCN2)OCCc2cc(I)sc23)nn1] | 33 |
| 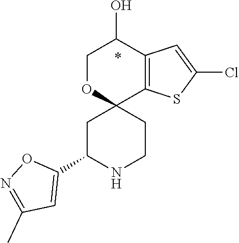 | 34 |
| 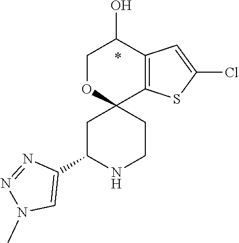 | 35 |
| 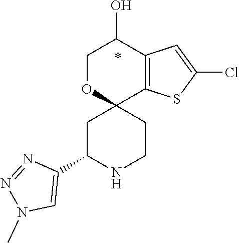 | 36 |
| 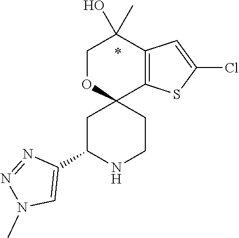 | 37 |
| 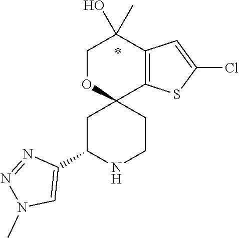 | 38 |
| 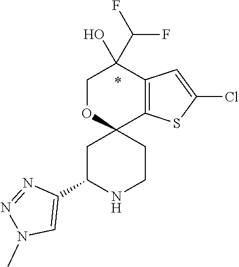 | 39 |
| 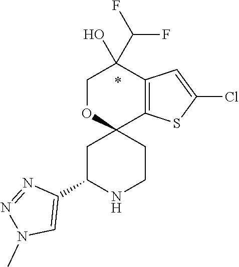 | 40 |
| 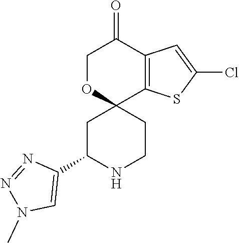 [SMILES: Cn1cc([C@@H]2C[C@]3(CCN2)OCC(=O)c2cc(Cl)sc23)nn1] | 41 |
| 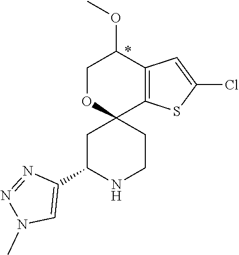 | 42 |
| 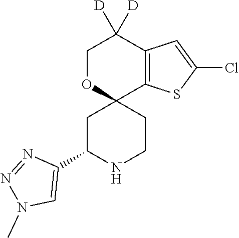 [SMILES: [2H]C1([2H])CO[C@]2(CCN[C@H](c3cn(C)nn3)C2)c2sc(Cl)cc21] | 43 |
| 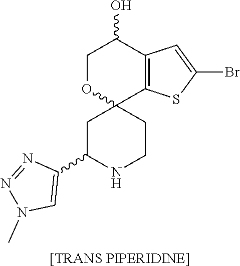 | 44 |
| 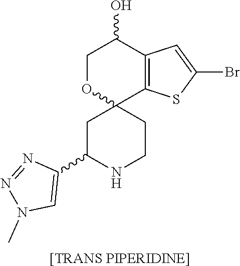 | 45 |
| 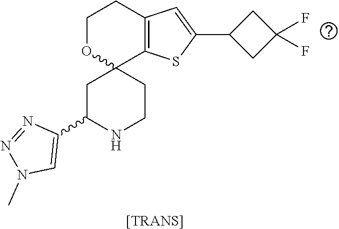 | 46 |
| 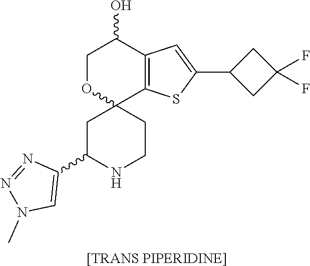 | 47 |
| 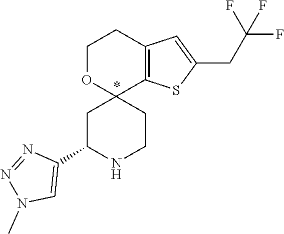 [SMILES: Cn1cc([C@@H]2CC3(CCN2)OCCc2cc(CC(F)(F)F)sc23)nn1] | 48 |
| 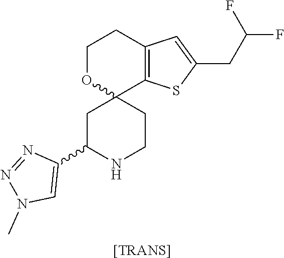 | 49 |
| 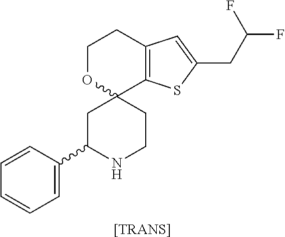 | 50 |
| 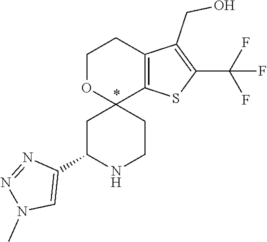 [SMILES: Cn1cc([C@@H]2CC3(CCN2)OCCc2c3sc(C(F)(F)F)c2CO)nn1] | 51 |
| 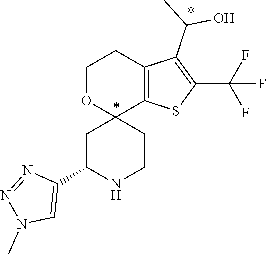 [SMILES: CC(O)c1c(C(F)(F)F)sc2c1CCOC21CCN[C@H](c2cn(C)nn2)C1] | 52 |
| 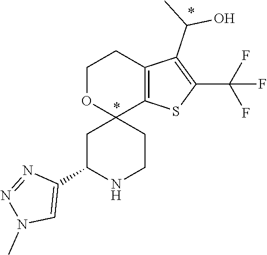 [SMILES: CC(O)c1c(C(F)(F)F)sc2c1CCOC21CCN[C@H](c2cn(C)nn2)C1] | 53 |
| 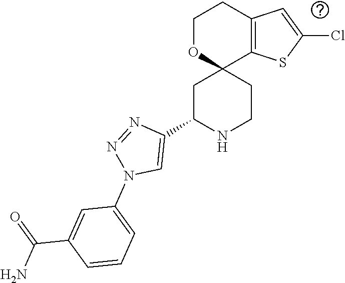 [SMILES: NC(=O)c1cccc(-n2cc([C@@H]3C[C@]4(CCN3)OCCc3cc(Cl)sc34)nn2)c1] | 54 |
| 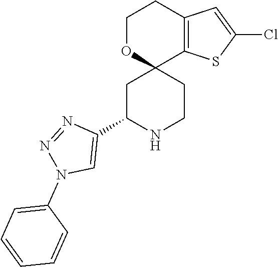 [SMILES: Clc1cc2c(s1)[C@@]1(CCN[C@H](c3cn(-c4ccccc4)nn3)C1)OCC2] | 55 |
| 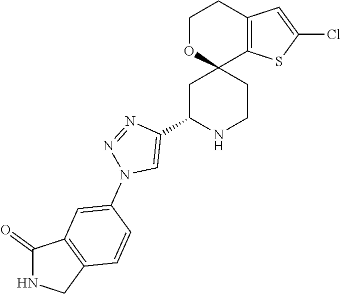 [SMILES: O=C1NCc2ccc(-n3cc([C@@H]4C[C@]5(CCN4)OCCc4cc(Cl)sc45)nn3)cc21] | 56 |
| 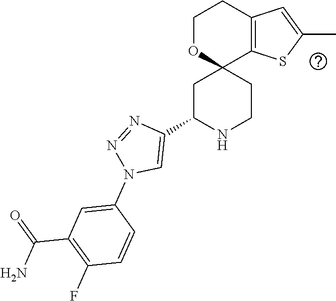 [SMILES: Cc1cc2c(s1)[C@@]1(CCN[C@H](c3cn(-c4ccc(F)c(C(N)=O)c4)nn3)C1)OCC2] | 57 |
| 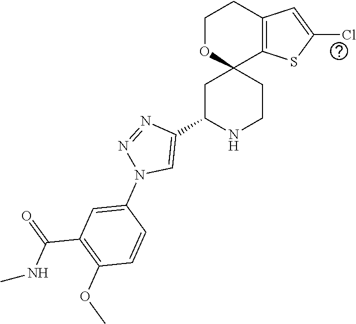 [SMILES: CNC(=O)c1cc(-n2cc([C@@H]3C[C@]4(CCN3)OCCc3cc(Cl)sc34)nn2)ccc1OC] | 58 |
| 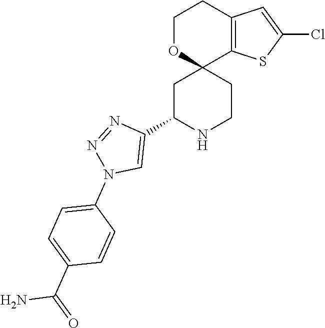 [SMILES: NC(=O)c1ccc(-n2cc([C@@H]3C[C@]4(CCN3)OCCc3cc(Cl)sc34)nn2)cc1] | 59 |
| 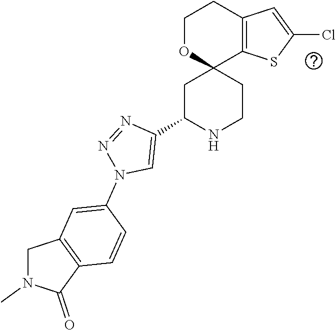 [SMILES: CN1Cc2cc(-n3cc([C@@H]4C[C@]5(CCN4)OCCc4cc(Cl)sc45)nn3)ccc2C1=O] | 60 |
| 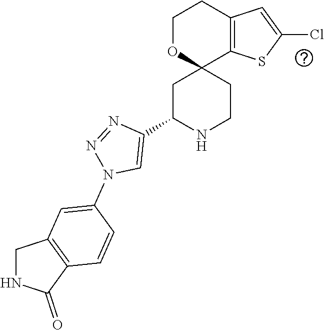 [SMILES: O=C1NCc2cc(-n3cc([C@@H]4C[C@]5(CCN4)OCCc4cc(Cl)sc45)nn3)ccc21] | 61 |
| 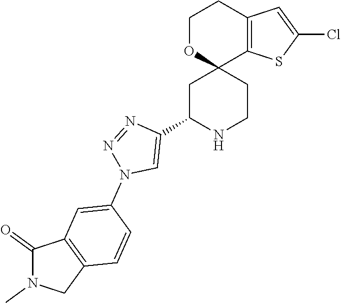 [SMILES: CN1Cc2ccc(-n3cc([C@@H]4C[C@]5(CCN4)OCCc4cc(Cl)sc45)nn3)cc2C1=O] | 62 |
| 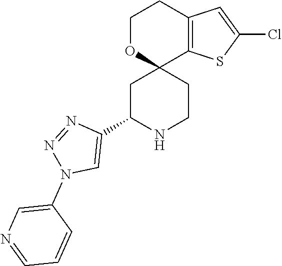 [SMILES: Clc1cc2c(s1)[C@@]1(CCN[C@H](c3cn(-c4cccnc4)nn3)C1)OCC2] | 63 |
| 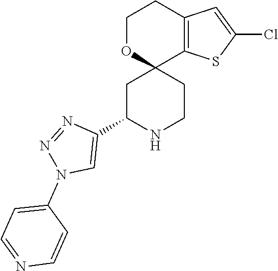 [SMILES: Clc1cc2c(s1)[C@@]1(CCN[C@H](c3cn(-c4ccncc4)nn3)C1)OCC2] | 64 |
| 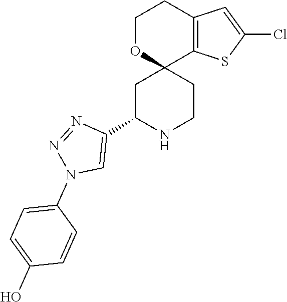 [SMILES: Oc1ccc(-n2cc([C@@H]3C[C@]4(CCN3)OCCc3cc(Cl)sc34)nn2)cc1] | 65 |
| 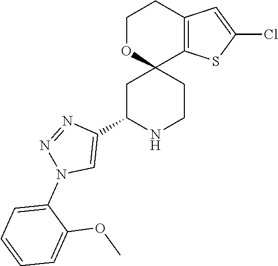 [SMILES: COc1ccccc1-n1cc([C@@H]2C[C@]3(CCN2)OCCc2cc(Cl)sc23)nn1] | 66 |
| 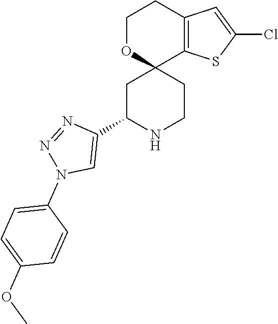 [SMILES: COc1ccc(-n2cc([C@@H]3C[C@]4(CCN3)OCCc3cc(Cl)sc34)nn2)cc1] | 67 |
| 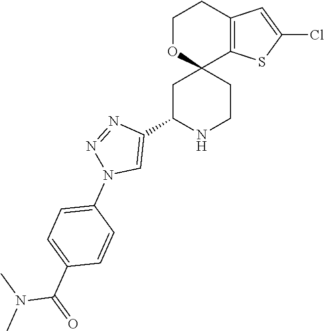 [SMILES: CN(C)C(=O)c1ccc(-n2cc([C@@H]3C[C@]4(CCN3)OCCc3cc(Cl)sc34)nn2)cc1] | 68 |
| 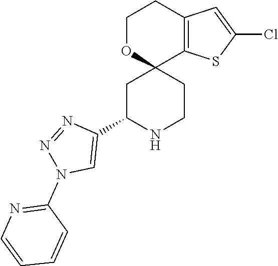 [SMILES: Clc1cc2c(s1)[C@@]1(CCN[C@H](c3cn(-c4ccccn4)nn3)C1)OCC2] | 69 |
| 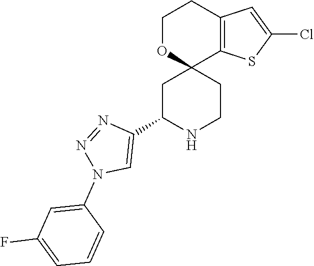 [SMILES: Fc1cccc(-n2cc([C@@H]3C[C@]4(CCN3)OCCc3cc(Cl)sc34)nn2)c1] | 70 |
| 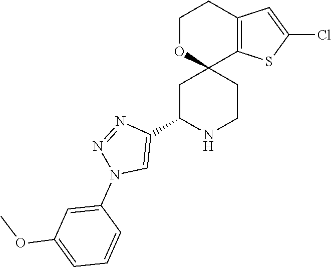 [SMILES: COc1cccc(-n2cc([C@@H]3C[C@]4(CCN3)OCCc3cc(Cl)sc34)nn2)c1] | 71 |
| 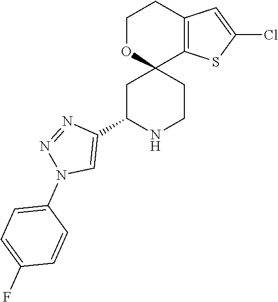 [SMILES: Fc1ccc(-n2cc([C@@H]3C[C@]4(CCN3)OCCc3cc(Cl)sc34)nn2)cc1] | 72 |
| 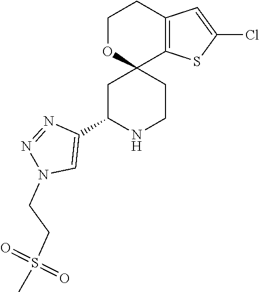 [SMILES: CS(=O)(=O)CCn1cc([C@@H]2C[C@]3(CCN2)OCCc2cc(Cl)sc23)nn1] | 73 |
| 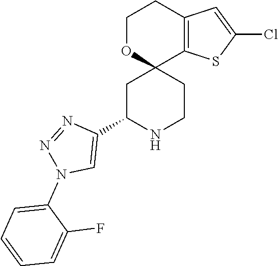 [SMILES: Fc1ccccc1-n1cc([C@@H]2C[C@]3(CCN2)OCCc2cc(Cl)sc23)nn1] | 74 |
| 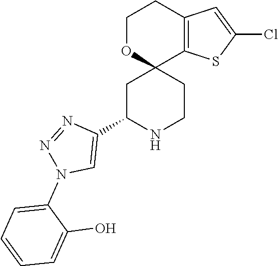 [SMILES: Oc1ccccc1-n1cc([C@@H]2C[C@]3(CCN2)OCCc2cc(Cl)sc23)nn1] | 75 |
| 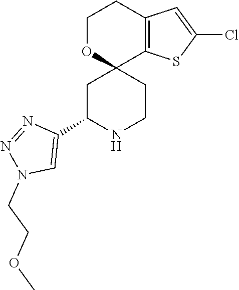 [SMILES: COCCn1cc([C@@H]2C[C@]3(CCN2)OCCc2cc(Cl)sc23)nn1] | 76 |
| 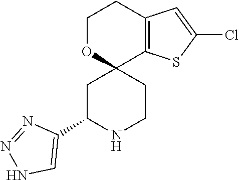 [SMILES: Clc1cc2c(s1)[C@@]1(CCN[C@H](c3c[nH]nn3)C1)OCC2] | 77 |
| 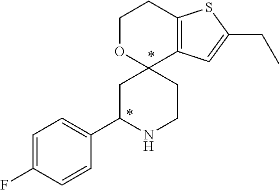 [SMILES: CCc1cc2c(s1)CCOC21CCNC(c2ccc(F)cc2)C1] | 78 |

Some embodiments of the disclosure include derivatives of Compounds 1 to 78 or compounds of Formulae I, IIa, IIb, IIc, IId, IIIa, IIIb, IIIc, and IIId, tautomers thereof, deuterated derivatives of those compounds or tautomers, or pharmaceutically acceptable salts of any of the foregoing. In some embodiments, the derivatives are silicon derivatives in which at least one carbon atom in a compound, tautomer, deuterated derivative, or pharmaceutically acceptable salt chosen from Compounds 1 to 78 or compounds of Formulae I, IIa, IIb, IIc, IId, IIa, IIIb, IIIc, and IIId, tautomers thereof, deuterated derivatives of those compounds or tautomers, and pharmaceutically acceptable salts of any of the foregoing, has been replaced by silicon. In some embodiments, the derivatives are boron derivatives, in which at least one carbon atom in a compound, tautomer, deuterated derivative, or pharmaceutically acceptable salt chosen from Compounds 1 to 78 or compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, has been replaced by boron. In other embodiments, the derivatives are phosphorus derivatives, in which at least one carbon atom in a compound, tautomer, deuterated derivative, or pharmaceutically acceptable salt chosen from Compounds 1 to 78 or compounds of Formulae Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, has been replaced by phosphorus.

In some embodiments, the derivative is a silicon derivative in which one carbon atom in a compound, tautomer, deuterated derivative, or pharmaceutically acceptable salt chosen from Compounds 1 to 78 or compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, has been replaced by silicon or a silicon derivative (e.g., —Si(CH₃)₂— or —Si(OH)₂—). The carbon replaced by silicon may be a non-aromatic carbon. In other embodiments, a fluorine has been replaced by silicon derivative (e.g., —Si(CH₃)₃). In some embodiments, the silicon derivatives of the disclosure may include one or more hydrogen atoms replaced by deuterium. In some embodiments, a silicon derivative of compound, tautomer, deuterated derivative, or pharmaceutically acceptable salt chosen from Compounds 1 to 78 or compounds of Formulae I, IIa, IIb, IIc, IId, IIIa, IIIb, IIIc, and IIId, a tautomer thereof, a deuterated derivative of that compound or tautomer, or a pharmaceutically acceptable salt of any of the foregoing, may have silicon incorporated into a heterocycle ring.

In some embodiments, the derivative is a boron derivative in which one carbon atom in a compound, tautomer, deuterated derivative, or pharmaceutically acceptable salt chosen from Compounds 1 to 78 or compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, has been replaced by boron or a boron derivative.

In some embodiments, the derivative is a phosphorus derivative in which one carbon atom in a compound, tautomer, deuterated derivative, or pharmaceutically acceptable salt chosen from Compounds 1 to 78 or compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing, has been replaced by phosphorus or a phosphorus derivative.

Another aspect of the disclosure provides pharmaceutical compositions comprising at least one compound, tautomer, deuterated derivative, or pharmaceutically acceptable salt according to any one formula chosen from Formulae I, IIa, IIb, IIc, IId, IIIa, IIIb, IIIc, and IIId, and Compounds 1 to 78, tautomers thereof, deuterated derivatives of those compounds or tautomers, and pharmaceutically acceptable salts of any of the foregoing. In some embodiments, the pharmaceutical composition comprising at least one compound, tautomer, deuterated derivative, or pharmaceutically acceptable salt chosen from Formulae I, IIa, IIb, IIc, IId, IIIa, IIIb, IIIc, and IIId, Compounds 1 to 78, tautomers thereof, deuterated derivatives of those compounds or tautomers, and pharmaceutically acceptable salts of any of the foregoing is administered to a patient in need thereof.

A pharmaceutical composition may further comprise at least one pharmaceutically acceptable carrier. In some embodiments, the at least one pharmaceutically acceptable carrier is chosen from pharmaceutically acceptable vehicles and pharmaceutically acceptable adjuvants. In some embodiments, the at least one pharmaceutically acceptable is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include at least one additional active therapeutic agent. Alternatively, a pharmaceutical composition comprising at least one compound, tautomer, deuterated derivative, or pharmaceutically acceptable salt chosen from Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent. In some embodiments, a pharmaceutical composition comprising at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing can be administered as a separate composition concurrently with, prior to, or subsequent to, a composition comprising at least one other active therapeutic agent.

As described above, pharmaceutical compositions disclosed herein may optionally further comprise at least one pharmaceutically acceptable carrier. The at least one pharmaceutically acceptable carrier may be chosen from adjuvants and vehicles. The at least one pharmaceutically acceptable carrier, as used herein, includes any and all solvents, diluents, other liquid vehicles, dispersion aids, suspension aids, surface active agents, isotonic agents, thickening agents, emulsifying agents, preservatives, solid binders, and lubricants, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include, but are not limited to, ion exchangers, alumina, aluminum stearate, lecithin, serum proteins (such as, e.g., human serum albumin), buffer substances (such as, e.g., phosphates, glycine, sorbic acid, and potassium sorbate), partial glyceride mixtures of saturated vegetable fatty acids, water, salts, and electrolytes (such as, e.g., protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as, e.g., lactose, glucose, and sucrose), starches (such as, e.g., corn starch and potato starch), cellulose and its derivatives (such as, e.g., sodium carboxymethyl cellulose, ethyl cellulose, and cellulose acetate), powdered tragacanth, malt, gelatin, talc, excipients (such as, e.g., cocoa butter and suppository waxes), oils (such as, e.g., peanut oil, cottonseed oil, safflower oil, sesame oil, olive oil, corn oil, and soybean oil), glycols (such as, e.g., propylene glycol and polyethylene glycol), esters (such as, e.g., ethyl oleate and ethyl laurate), agar, buffering agents (such as, e.g., magnesium hydroxide and aluminum hydroxide), alginic acid, pyrogen-free water, isotonic saline, Ringer's solution, ethyl alcohol, phosphate buffer solutions, non-toxic compatible lubricants (such as, e.g., sodium lauryl sulfate and magnesium stearate), coloring agents, releasing agents, coating agents, sweetening agents, flavoring agents, perfuming agents, preservatives, and antioxidants.

### Use of Compounds and Compositions

In some embodiments of the disclosure, the compounds and the pharmaceutical compositions described herein are used to treat FSGS and/or NDKD. In some embodiments, FSGS is mediated by APOL1. In some embodiments, NDKD is mediated by APOL1.

In some embodiments of the disclosure, the compounds and the pharmaceutical compositions described herein are used to treat cancer. In some embodiments, the cancer is mediated by APOL1.

In some embodiments of the disclosure, the compounds and the pharmaceutical compositions described herein are used to treat pancreatic cancer. In some embodiments, the pancreatic cancer is mediated by APOL1.

In some embodiments, the methods of the disclosure comprise administering to a patient in need thereof at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds and tautomers, and pharmaceutically acceptable salts of any of the foregoing. In some embodiments, the compound, tautomer, deuterated derivative, or pharmaceutically acceptable salt is chosen from Compounds 1 to 78, tautomer thereof, deuterated derivatives of those compounds or tautomers, and pharmaceutically acceptable salts of any of the foregoing. In some embodiments, said patient in need thereof possesses APOL1 genetic variants, i.e., G1: S342G:1384M and G2: N388del:Y389del.

Another aspect of the disclosure provides methods of inhibiting APOL1 activity comprising contacting said APOL1 with at least one compound, tautomer, deuterated derivative, or pharmaceutically acceptable salt chosen from compounds of Formulae I, IIa, IIb, IIc, IId, IIIa, IIIb, IIIc, and IIId, tautomers thereof, deuterated derivatives of those compounds or tautomers, and pharmaceutically acceptable salts of any of the foregoing. In some embodiments, the methods of inhibiting APOL1 activity comprise contacting said APOL1 with at least one compound, tautomer, deuterated derivative, or pharmaceutically acceptable salt chosen from Compounds 1 to 78, tautomers thereof, deuterated derivatives of those compounds or tautomers, and pharmaceutically acceptable salts of any of the foregoing.

### EXAMPLES

In order that the disclosure described herein may be more fully understood, the following examples are set forth. It should be understood that these examples are for illustrative purposes only and are not to be construed as limiting this disclosure in any manner.

The compounds of the disclosure may be made according to standard chemical practices or as described herein. Throughout the following synthetic schemes and in the descriptions for preparing compounds of Formulae I, IIa, IIb, IIc, IId, IIIa, IIIb, IIIc, and IIId, Compounds 1 to 78, tautomers thereof, deuterated derivatives of those compounds and tautomers, and pharmaceutically acceptable salts of any of the foregoing, the following abbreviations are used:

### Abbreviations

AcOH=acetic acid
ARP=assay ready plate
Boc₂O=di-tert-butyl dicarbonate
CBS=Corey-Bakshi-Shibata
CDMT=2-chloro-4,6-dimethoxy-1,3,5-triazine
Co(OAc)₂=Cobalt(II) acetate
DCM=dichloromethane
DIBAL-H=diisobutylaluminum hydride
DIPEA=N,N-Diisopropylethylamine or N-ethyl-N-isopropyl-propan-2-amine
DMAP=dimethylamino pyridine
DME=dimethoxyethane
DMEM=Dulbecco's modified Eagle's medium
DMF=dimethylformamide
DMPU=N,N′-dimethylpropyleneurea
DMSO=dimethyl sulfoxide
ESI-MS=electrospray ionization mass spectrometry,
EtOAc=ethyl acetate
EtOH=ethanol
FBS=fetal bovine serum
GCMS=gas chromatography mass spectrometry
HPLC=high-performance liquid chromatography
IPA=isopropyl alcohol
Ir[df(CF₃)ppy]₂(dtbbpy)PF₆=[4,4′-Bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate
LED=light emitting diode
LiTMP=Lithium tetramethylpiperidide
MeCN or ACN=acetonitrile
MeI=methyl iodide
MeMgBr=methylmagnesium bromide
MeMgCl=methylmagnesium chloride
MeOAc=methyl acetate
MeOH=methanol
MsOH=methanesulfonic acid
MTBE=Methyl tert-butyl ether
n-BuLi=n-butyllithium
NBS=n-bromosuccinimide
NHPI=N-hydroxyphthalimide,
NIS=N-iodosuccinimide
NMR=nuclear magnetic resonance
Pd(dppf)₂Cl₂=[1,1′-Bis(diphenylphosphino)ferrocene]dichloropalladium(II)
PP=polypropylene
PPh₃=triphenylphosphine
PTSA=p-Toluenesulfonic acid monohydrate
SFC=supercritical fluid chromatography
TBAF=tetra-n-butylammonium fluoride
TBS=tert-butyldimethylsilyl
TEA=triethylamine
Tet=tetracycline
TFA or TFAA=trifluoroacetic acid
TfOH=triflic acid
THF=tetrahydrofuran
2-MeTHF=2-methyltetrahydrofuran
TLC=thin layer chromatography
TMS=tetramethylsilane
TMSCF₂Br=(Bromodifluoromethyl)trimethylsilane
TMSCl=trimethylsilyl chloride

### Example 1. Synthesis of Compounds

All the specific and generic compounds, and the intermediates disclosed for making those compounds, are considered to be part of the disclosure disclosed herein.

### Preparation S1

### 2-(3-thienyl)ethanol (S1)

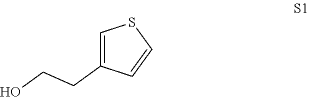

2-(3-thienyl)ethanol (S1) was obtained from commercial sources.

### Preparation S2

### 2-(5-chloro-3-thienyl)ethanol (S2)

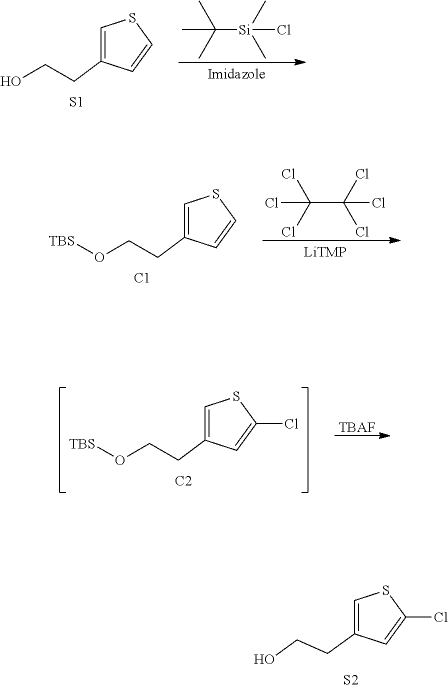

### Step 1. Synthesis of tert-butyl-dimethyl-[2-(3-thienyl)ethoxy]silane (C1)

To a solution of 2-(3-thienyl)ethanol (18 g, 140.4 mmol) in DMF (100 mL) was added imidazole (12 g, 176.3 mmol) and tert-butyl-chloro-dimethyl-silane (24 g, 159.2 mmol) sequentially. Exotherm was observed. The reaction mixture was stirred at room temperature for 3 hours. Reaction had stalled at 90% conversion. Reaction was diluted with MTBE (500 mL) and washed with water (200 mL), 0.5 M HCl, (200 mL), water (200 mL), and brine (200 mL). The organic layer was dried, filtered and concentrated in vacuo. The organic layer was dissolved in heptane and passed through a silica gel plug; which was washed with 1-5% MTBE/Heptane. Solvent was removed to afford tert-butyl-dimethyl-[2-(3-thienyl)ethoxy]silane C1 (34 g, 99%). ¹H NMR (400 MHz, Chloroform-d) δ 7.28-7.13 (m, 1H), 7.04-6.91 (m, 2H), 3.80 (t, J=6.9 Hz, 2H), 2.90-2.75 (m, 2H), 0.88 (s, 9H), −0.00 (s, 6H).

### Step 2. Synthesis of tert-butyl-[2-(5-chloro-3-thienyl)ethoxy]-dimethyl-silane (C2)

To a solution of 2,2,6,6-tetramethylpiperidine (36 mL, 213.3 mmol) in tetrahydrofuran (200 mL) cooled to 0° C.; was added a solution of hexyllithium (92 mL of 2.3 M, 211.6 mmol). Reaction was stirred for 30 minutes at −78° C. A solution of tert-butyl-dimethyl-[2-(3-thienyl)ethoxy]silane (34 g, 138.8 mmol) in THF (150 mL) was added to the reaction over 20 minutes. The reaction was stirred at −30° C. for 45 minutes. The reaction was cooled to −78° C. 1,1,1,2,2,2-hexachloroethane (54 g, 228.1 mmol) was added portion-wise. The reaction was warmed to room temperature and stirred overnight. The reaction was quenched with saturated ammonium chloride (125 mL), diluted with water (100 mL), extracted with EtOAc (500 mL), and back extracted with EtOAc (100 mL). The combined organic layers were washed with 0.5 M HCl (200 mL), water (300 mL), and brine (200 mL). Organic layer was dried over sodium sulfate, filtered and concentrated to afford the crude product (C2).

### Step 3. Synthesis of 2-(5-chloro-3-thienyl)ethanol (S2)

To a solution of tert-butyl-[2-(5-chloro-3-thienyl)ethoxy]-dimethyl-silane C2 (12.5 g, 42.89 mmol) in 2-Me-THF (120 mL) was added TBAF (63 mL of 1 M, 63.00 mmol) (solution in THF). Reaction was stirred at room temperature overnight. The reaction was partitioned between EtOAc (400 mL) and water (400 mL). The layers were separated and the organic layer was extracted with EtOAc (200 mL), dried over sodium sulfate, filtered, and concentrated in vacuo. Purification by silica gel chromatography (Gradient: 0-50% EtOAc in heptane) yielded the product 2-(5-chloro-3-thienyl)ethanol S2 (4.5 g, 58%). ¹H NMR (300 MHz, Chloroform-d) δ 6.82 (d, J=0.9 Hz, 2H), 3.89-3.71 (m, 2H), 2.79 (t, J=6.4 Hz, 2H), 2.05 (s, 1H). LCMS m/z 162.91 [M+H]⁺.

### Preparation S3

### 2-[5-(trifluoromethyl)-3-thienyl]ethanol (S3)

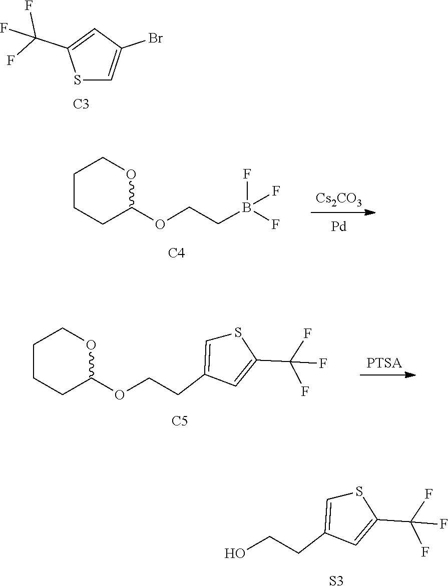

### Step 1. Synthesis of 2-[2-[5-(trifluoromethyl)-3-thienyl]ethoxy]tetrahydropyrane (C5)

To a mixture of 4-bromo-2-(trifluoromethyl)thiophene (C3) (9 g, 38.96 mmol), dicyclohexyl-[2-(2,6-diisopropoxyphenyl)phenyl]phosphane; methanesulfonate; N-methyl-2-phenyl-aniline palladium (2+) (1.8 g, 2.117 mmol), and potassium trifluoro(2-tetrahydropyran-2-yloxyethyl)boranuide C4 (10 g, 42.36 mmol) was added toluene (75 mL) and water (25 mL). Nitrogen was passed over the top of the reaction before addition of Cs₂CO₃ (40 g, 122.8 mmol). A reflux condenser was added and the reaction was heated at 100° C. for 48 hours. The reaction was diluted with EtOAc (150 mL) and water (100 mL). The two layers were separated and the aqueous layered was extracted with EtOAc (100 mL). The combined organics were washed with brine, dried over sodium sulfate, filtered, and concentrated in vacuo. Purification by silica gel chromatography (Gradient: 0-20% EtOAc in heptane) yielded the product. 2-[2-[5-(trifluoromethyl)-3-thienyl]ethoxy]tetrahydropyran C5 (9 g, 82%). ¹H NMR (300 MHz, Chloroform-d) δ 7.37 (t, J=1.3 Hz, 1H), 7.22 (d, J=1.5 Hz, 1H), 4.62 (dd, J=4.2, 2.8 Hz, 1H), 3.96 (dt, J=9.6, 6.7 Hz, 1H), 3.75 (ddd, J=11.3, 8.0, 3.4 Hz, 1H), 3.62 (dt, J=9.6, 6.5 Hz, 1H), 3.55-3.41 (m, 1H), 2.93 (t, J=6.6 Hz, 2H), 1.83 (ddd, J=14.2, 6.6, 3.4 Hz, 1H), 1.73 (td, J=9.0, 4.2 Hz, 1H), 1.66-1.50 (m, 4H).

### Step 2. Synthesis of 2-[5-(trifluoromethyl)-3-thienyl]ethanol (S3)

To a stirred solution of 2-[2-[5-(trifluoromethyl)-3-thienyl]ethoxy]tetrahydropyran C5 (1.8 g, 6.100 mmol) in MeOH (25 mL) was added 4-methylbenzenesulfonic acid (Water (1)) (1.2 g, 6.309 mmol) at room temperature. The reaction mixture was stirred at room temperature for 1 hour. The reaction mixture was diluted with water (100 mL) and extracted with MTBE (2×100 mL). The combined organic layers were washed with dilute NaHCO₃ (10 mL NaHCO₃ and 10 mL water) and brine (10 mL), dried over sodium sulfate, filtered, and evaporated under vacuum to get crude compound. Purification by silica gel chromatography (Gradient: 0-30% EtOAc in heptane) yielded the product 2-[5-(trifluoromethyl)-3-thienyl]ethanol S3 (820 mg, 69%). ¹H NMR (400 MHz, Chloroform-d) δ 7.35 (p, J=1.3 Hz, 1H), 7.23 (dt, J=1.7, 0.9 Hz, 1H), 3.85 (td, J=7.1, 6.5, 2.7 Hz, 2H), 2.87 (td, J=6.4, 0.8 Hz, 2H), 2.06 (d, J=4.3 Hz, 1H).

### Preparation S4

### 2-(5-bromo-3-thienyl)ethanol (S4)

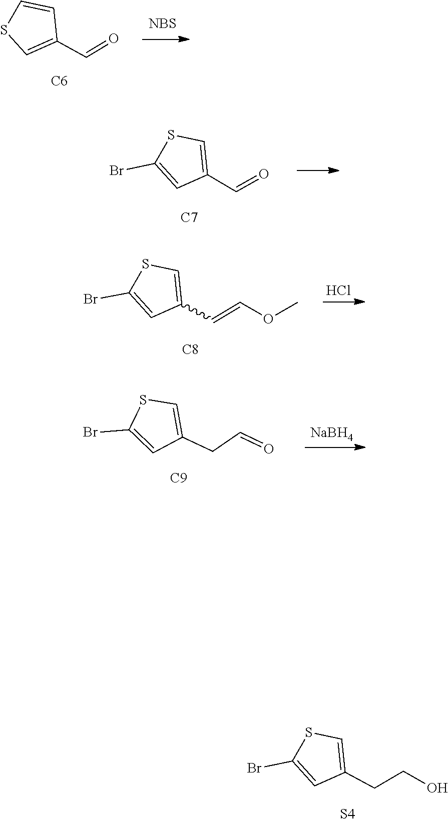

### Step 1. Synthesis of 5-bromothiophene-3-carbaldehyde (C7)

To a stirred solution of thiophene-3-carbaldehyde C₆ (50 g, 40.717 mL, 0.4458 mol) in DMF (500 mL) was added NBS (119.02 g, 0.6687 mol) at 0° C. The reaction mixture was stirred at room temperature for 16 hours. Reaction mixture was quenched with ice cold water (600 mL) and extracted with EtOAc (2×600 mL). Combined organic layers were dried over Na₂SO₄, filtered, and concentrated. Purification by silica gel chromatography (Gradient: 0-2% EtOAc in Petroleum ether) yielded the product 5-bromothiophene-3-carbaldehyde C7 (39.2 g, 44%). ¹H NMR (400 MHz, Chloroform-d) δ 9.77 (s, 1H), 7.99 (d, J=1.2 Hz, 1H), 7.505 (d, J=1.6 Hz, 1H).

### Step 2. Synthesis of 2-bromo-4-[(E)-2-methoxyvinyl]thiophene (C8)

To a stirred solution of (Methoxymethyl)triphenylphosphonium Chloride (115.1 g, 0.3358 mol) in Diethyl Ether (450.00 mL) at 0° C. was added Potassium tert-butoxide (1 M in THF) (381 mL of 1 M, 0.3810 mol) drop-wise. The reaction was stirred at 0° C. for 1 hour. A solution of 5-bromothiophene-3-carbaldehyde C7 (45 g, 0.2215 mol) in Diethyl Ether (90 mL) was added, and then the reaction mixture was stirred at room temperature for 30 minutes. Reaction mixture was quenched with NH₄Cl solution (900 mL) at 0° C., extracted with EtOAc (2×700 mL). The combined organic layers were dried over Na₂SO₄, filtered and concentrated. Purification by silica gel chromatography (Eluent: Petroleum ether) afforded the product, 2-bromo-4-[(E)-2-methoxyvinyl]thiophene C8 (44.1 g, 82%). ¹H NMR (400 MHz, Chloroform-d) δ 7.25 (d, J=2 Hz, 1H), 7.18 (d, J=0.8 Hz, 1H), 7.00 (d, J=1.8 Hz, 1H), 6.91 (d, J=12.8 Hz, 1H), 6.97 (d, J=1.2 Hz, 1H), 6.05 (d, J=6.8 Hz, 1H), 5.72 (d, J=12.8 Hz, 1H), 5.22 (d, J=6.4 Hz, 1H), 3.77 (d, J=2.8 Hz, 3H), 3.64 (d, J=5.2 Hz, 3H). NMR showed a 1:1 mixture of E and Z isomers.

### Step 3. Synthesis of 2-(5-bromo-3-thienyl)acetaldehyde (C9)

To a stirred solution of 2-bromo-4-[(E)-2-methoxyvinyl]thiophene C8 (14.1 g, 0.0602 mol) in 1,4-Dioxane (141.00 mL) was added HCl (4 M in Dioxane) (60.200 mL of 4 M, 0.2408 mol) at 0° C. The reaction mixture was stirred at room temperature for 30 minutes. Reaction mixture was quenched with saturated NaHCO₃ at 0° C. and extracted with EtOAc. The organic layer was dried over Na₂SO₄, filtered and concentrated, to afford 2-(5-bromo-3-thienyl)acetaldehyde C9 (13.1 g, 89%). ¹H NMR (400 MHz, Chloroform-d) δ 9.72 (t, J=2.4 Hz, 1H), 7.04 (s, 1H), 6.94 (d, J=1.2 Hz, 1H), 3.66 (d, J=1.6 Hz, 2H).

### Step 4. Synthesis of 2-(5-bromo-3-thienyl)ethanol (S4)

To a stirred solution of 2-(5-bromo-3-thienyl)acetaldehyde C9 (38.5 g, 0.1524 mol) in MeOH (390 mL) was added NaBH₄ (13.3 g, 0.3515 mol) at 0° C. Reaction was stirred for 1 hour. The reaction mixture was quenched with ice water (400 mL) and concentrated in vacuo to remove the MeOH. The crude residue was diluted with water (500 mL) and extracted with EtOAc (3×300 mL). The separated organic layers were dried over Na₂SO₄, filtered and concentrated. Purification by column chromatography with neutral alumina (Eluent: 35% EtOAc in petroleum ether) afforded the product 2-(5-bromo-3-thienyl)ethanol S4 (30.2 g, 84%). ¹H NMR (300 MHz, DMSO-d₆) δ 7.20 (t, J=0.9 Hz, 1H), 7.10 (d, J=1.2 Hz, 1H), 4.64 (q, J=5.2 Hz, 1H), 3.59-3.55 (m, 2H), 2.67 (t, J=6.8 Hz, 2H) as a pale yellow liquid.

### Preparation S5

### 2-(5-ethyl-3-thienyl)ethanol (S5)

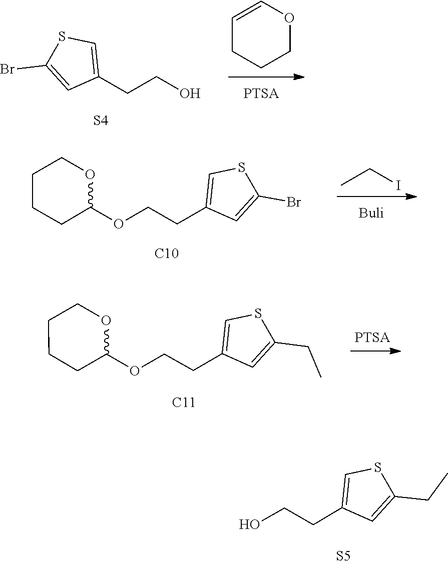

### Step 1. Synthesis of 2-[2-(5-bromo-3-thienyl)ethoxy]tetrahydropyran (C10)

To a stirred solution of 2-(5-bromo-3-thienyl)ethanol S4 (8 g, 0.0328 mol) in THF (80. mL) was added 3,4-Dihydro-2H-pyran (3.7696 g, 3.8 mL, 0.0448 mol) and PTSA (259 mg, 0.0015 mol) at room temperature. Then reaction mixture was stirred at room temperature for 16 hours. The reaction mixture was quenched with saturated aq. K₂CO₃ (300 mL), and extracted with EtOAc (2×600 mL). The organic layers were dried over Na₂SO₄, filtered, and concentrated. Purification by silica gel chromatography (Gradient: 0-5% EtOAc in Petroleum ether) yielded the product 2-[2-(5-bromo-3-thienyl)ethoxy]tetrahydropyran C10 (10.1 g, 90%). ¹H NMR (400 MHz, Chloroform-d) δ 6.95 (d, J=1.6 Hz, 1H), 6.92 (d, J=0.8, 1H), 4.59 (t, J=2.8 Hz, 1H), 3.94-3.74 (m, 2H), 3.60-3.46 (m, 2H), 2.85 (q, J=6.4 Hz, 2H), 1.80-1.61 (m, 6H). LCMS m/z 291.03 [M+H]⁺.

### Step 6. Synthesis of 2-[2-(5-ethyl-3-thienyl)ethoxy]tetrahydropyran (C11)

To a stirred solution of 2-[2-(5-bromotetrahydrothiophen-3-yl)ethoxy]tetrahydropyran C10 (25 g, 0.0719 mol) in THF (250.00 mL) was added n-BuLi (2.5 M in Hexane) (46.1 mL of 2.5 M, 0.1153 mol) at −76° C. Reaction was stirred for 1 hour. Ethyl iodide (24.832 g, 12.8 mL, 0.1592 mol) was added at −76° C. Then reaction temperature was slowly increased to room temperature, and was then stirred for 16 hours. The reaction mixture was quenched with NH₄Cl solution (500 mL), and extracted with EtOAc (2×300 mL). The combined organic layers were dried over Na₂SO₄, filtered and concentrated. Purification by silica gel chromatography (Gradient: 0-3% EtOAc in Petroleum ether) yielded the product 2-[2-(5-ethyl-3-thienyl)ethoxy]tetrahydropyran C11 (13.2 g, 59%). LCMS m/z 241.21 [M+H]⁺.

### Step 7. Synthesis of 2-(5-ethyl-3-thienyl)ethanol (S5)

To a stirred solution of 2-[2-(5-ethyl-3-thienyl)ethoxy]tetrahydropyran C11 (4.4 g, 0.0142 mol) in MeOH (44 mL) was added PTSA (3.0 g, 0.0174 mol) at room temperature and the reaction was stirred for 2 hours. Reaction mixture was quenched with saturated NaHCO₃ solution (150 mL), extracted with EtOAc (2×150 mL), dried over Na₂SO₄, filtered, and concentrated. Purification by column chromatography with neutral alumina (Eluent: 10% EtOAc in petroleum ether) afforded the product 2-(5-ethyl-3-thienyl)ethanol S5 (1.1 g, 45%). ¹H NMR (400 MHz, DMSO-d₆) δ 6.90 (d, J=1.2 Hz, 1H), 6.71 (d, J=1.2 Hz, 1H), 4.62-4.58 (m, 1H), 3.59-3.55 (m, 2H), 2.77-2.71 (m, 2H), 2.64 (t, J=7.2, 2H), 1.22-1.85 (m, 3H).

### Preparation S6

### 2-(5-ethyl-2-thienyl)ethanol (S6)

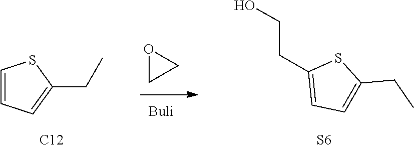

### Step 1. Synthesis of 2-(5-ethyl-2-thienyl)ethanol (S6)

To a solution of 2-ethylthiophene C12 (54 g, 466.9 mmol) in anhydrous THF (1 L) at 0° C. was added n-BuLi in hexane (255 mL of 2.2 M, 561.0 mmol) over 45 minutes. A light yellow/orange solution resulted. The temperature range during the addition was 0-10° C. The mixture was stirred at room temperature for 30 minutes. After cooling to 0° C., a solution of ethylene-oxide (200 mL of 2.9 M, 580.0 mmol) was added over 30 minutes. The reaction was stirred at 0° C. for 2 hours and then was warmed to room temperature. Reaction mixture was quenched with water (700 mL) and saturated NH₄Cl (200 mL) and the THF was evaporated. The product was extracted with EtOAc (1×400 mL; 2×150 mL). The combined organic layers were dried over anhydrous sodium sulfate, filtered, and concentrated in vacuo. The organic layer was passed through a silica gel plug washing with DCM (1000 mL), 80% EtOAc/Heptane (2×200 mL) and DCM (2×250 mL) to afford 2-(5-ethyl-2-thienyl)ethanol S6 (71.25 g, 93%). ¹H NMR (300 MHz, Chloroform-d) δ 6.69 (dt, J=3.4, 0.9 Hz, 1H), 6.64 (dt, J=3.3, 1.0 Hz, 1H), 3.84 (t, J=6.3 Hz, 2H), 3.08-2.97 (m, 2H), 2.82 (qd, J=7.5, 1.0 Hz, 2H), 1.31 (t, J=7.5 Hz, 4H).

### Preparation S7

### 2-[5-(trifluoromethyl)-2-thienyl]ethanol (S7)

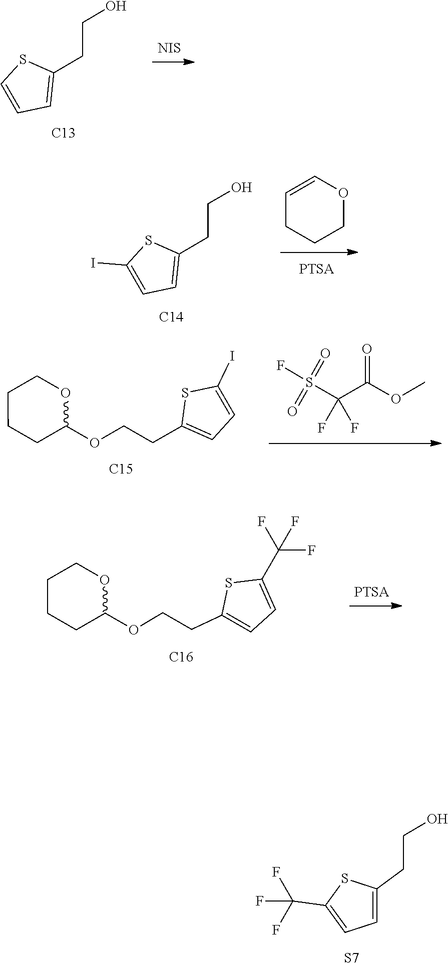

### Step 1. Synthesis of 2-(5-iodo-2-thienyl)ethanol (C14)

To a stirred solution of NIS (104.83 g, 0.4680 mol) in DCM (1000 mL) was added 2-(2-thienyl)ethanol C13 (50 g, 0.3900 mol) at 0° C. Reaction was warmed to room temperature and stirred for 16 hours. The reaction mixture was diluted with DCM (500 mL), washed with sat. sodium thiosulphate, brine, dried over Na₂SO₄ and concentrated in vacuo. Purification by column chromatography (Eluent: 20% EtOAc in petroleum ether) afforded the product 2-(5-iodo-2-thienyl)ethanol C14 (62 g, 56%). ¹H NMR (400 MHz, Chloroform-d) δ 7.08 (d, J=3.6 Hz, 1H), 6.57-6.56 (m, 1H), 3.82 (q, J=6 Hz, 2H), 3.05 (q, J=6.4 Hz, 2H). LCMS m/z 254.89 [M+H]⁺.

### Step 2. Synthesis of 2-[2-(5-iodo-2-thienyl)ethoxy]tetrahydropyran (C15)

To a stirred solution of 2-(5-iodo-2-thienyl)ethanol C14 (15 g, 0.0525 mol) and 3,4-dihydro-2H-pyran (6.6284 g, 0.0788 mol) in THF (60 mL) was added PTSA (1.3604 g, 1.2714 mL, 0.0079 mol) at room temperature. Reaction was stirred for 16 hours under argon balloon pressure. The reaction mixture was concentrated under reduced pressure. Purification by silica gel chromatography (Eluent: 5% EtOAc in Petroleum ether) yielded the product 2-[2-(5-iodo-2 thienyl)ethoxy]tetrahydropyran C15 (12.8 g, 68%). ¹H NMR (400 MHz, DMSO-d₆) δ 7.14 (d, J=3.6 Hz, 1H), 6.64 (d, J=3.6 Hz, 1H), 4.59 (t, J=3.6 Hz, 1H), 3.80-3.76 (m, 1H), 3.74-3.67 (m, 1H), 3.54-3.50 (m, 1H), 3.48-3.41 (m, 1H), 3.03 (t, J=6 Hz, 2H), 1.75-1.69 (m, 1H), 1.61-1.59 (m, 1H), 1.51-1.42 (m, 4H).

### Step 3. Synthesis of 2-[2-[5-(trifluoromethyl)-2-thienyl]ethoxy]tetrahydropyran (C16)

To a stirred solution of 2-[2-(5-iodo-2-thienyl)ethoxy]tetrahydropyran C15 (10 g, 0.0219 mol) and methyl 2,2-difluoro-2-fluorosulfonyl-acetate (12.63 g, 0.0657 mol) in DMF (40 mL) was added Copper(I) bromide dimethyl sulfide complex 99% (2.241 g, 0.0109 mol). Reaction was stirred at 100° C. for 16 hours. The reaction was warmed to room temperature, diluted with EtOAc (100 mL), filtered, and washed with EtOAc (50 mL). The filtrates were washed with chilled brine solution, dried over Na₂SO₄, and concentrated under reduced pressure. Purification by column chromatography with neutral alumina (Eluent: 5% EtOAc in petroleum ether) afforded the product C16 2-[2-[5-(trifluoromethyl)-2-thienyl]ethoxy]tetrahydropyran (2.9 g, 41%). ¹H NMR (400 MHz, Chloroform-d) δ 7.25 (s, 1H), 6.82-6.81 (m, 1H), 4.63 (t, J=3.6 Hz, 1H), 4.00-3.95 (m, 1H), 3.78-3.75 (m, 1H), 3.64-3.58 (m, 1H), 3.51-3.48 (m, 1H), 3.12 (d, J=6.4 Hz, 2H), 1.90-1.80 (m, 1H), 1.73-1.64 (m, 1H), 1.65-1.51 (m, 4H). GCMS m/z 280 [M]⁺.

### Step 4. Synthesis of 2-[5-(trifluoromethyl)-2-thienyl]ethanol (S7)

To a stirred solution of 2-[2-[5-(trifluoromethyl)-2-thienyl]ethoxy]tetrahydropyran C16 (5.8 g, 0.0170 mol) in MeOH (100 mL) was added PTSA (2.93 g, 0.0170 mol) at room temperature. Reaction was stirred for 16 hours. The reaction mixture was concentrated under reduced pressure. Purification by column chromatography with neutral alumina (Eluent: 10% EtOAc in petroleum ether) afforded the product 2-[5-(trifluoromethyl)-2-thienyl]ethanol S7 (2.3 g, 61%). ¹H NMR (400 MHz, DMSO-d₆) δ 7.52-7.51 (m, 1H), 6.99-6.98 (m, 1H), 4.92 (t, J=4.8 Hz, 1H), 3.65-3.61 (m, 2H), 2.98 (t, J=6 Hz, 2H). ¹⁹F NMR (376.22 MHz, DMSO-d₆) δ −53.53 (s, 3F). GCMS m/z 196.0 [M]⁺.

### Preparation S8 and S9

### 2-[5-(chloro)-2-thienyl]propan-1-ol (S8 [ENANT-1], S9 [ENANT-2])

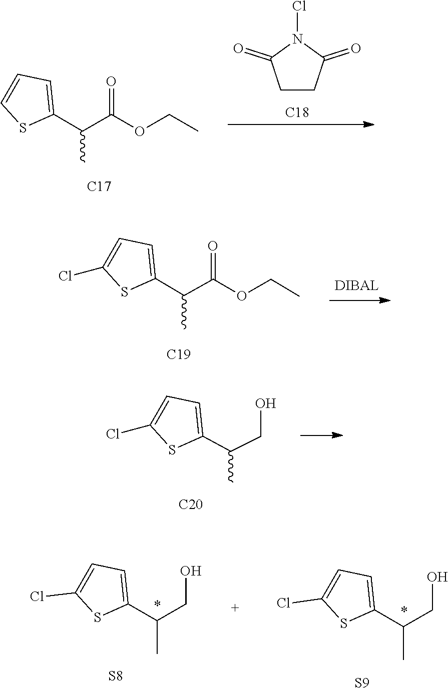

### Step 1. Synthesis of ethyl 2-(5-chloro-2-thienyl)propanoate (C19)

To a stirred solution of ethyl 2-(2-thienyl)propanoate C17 (1 g, 4.1139 mmol) in Acetic acid (10 mL) was added N-Chlorosuccinimide C18 (549.34 mg, 4.1139 mmol). The reaction mixture was stirred for 1 hour at 100° C. The mixture was concentrated and the resulting crude was diluted with EtOAc (25 mL), washed with water (10 mL), saturated sodium bicarbonate solution (10 mL), saturated sodium thiosulfate solution (10 mL), and brine solution (10 mL). The organic layer was dried over Na₂SO₄, filtered, and concentrated to afford crude product. Purification by silica gel chromatography (Eluent: 3% EtOAc in Petroleum ether) yielded the product ethyl 2-(5-chloro-2-thienyl)propanoate C19 (700 mg, 60%). ¹H NMR (Chloroform-d, 400 MHz): δ=6.75-6.73 (m, 1H), 6.71-6.69 (m, 1H), 4.20-4.14 (m, 2H), 3.88-3.73 (q, J=6.4 Hz, 1H), 1.55-1.53 (t, J=2.8 Hz, 3H), 1.30-1.221 (m, 3H). GCMS m/z 218.0 [M]⁺

### Step 2. Synthesis of 2-(5-chloro-2-thienyl)propan-1-ol (C20)

To a stirred solution of ethyl 2-(5-chloro-2-thienyl)propanoate C19 (25 g, 86.877 mmol) in THF (500 mL) was added DIBAL-H (74.135 mL of 25% w/v, 130.32 mmol) drop-wise at 0° C. The reaction mixture was stirred for 2 hours at 0° C. The mixture was slowly quenched with saturated NH₄Cl solution (300 mL) at 0° C. and the suspension was filtered through Celite® and the Celite® pad was washed with EtOAc (2×200 mL). The filtrate was separated into two layers. The aqueous layer was extracted with EtOAc (2×200 mL). The combined organic layers were washed with brine (200 mL), dried over sodium sulfate and concentrated. Purification by silica gel chromatography (Eluent: 3% EtOAc in Petroleum ether) yielded 2-(5-chloro-2-thienyl)propan-1-ol C20 (12 g, 72%). ¹H NMR (Chloroform-d, 400 MHz) δ 6.76-6.75 (d, J=3.6 Hz, 1H), 6.66-6.65 (dd, J=4.4 Hz, 1H), 3.71-3.61 (m, 2H), 3.15-3.10 (m, 1H), 1.57-1.52 (m, 1H), 1.34-1.31 (t, J=6 Hz, 3H). GCMS m/z 176.0 [M]⁺.

### Step 3. Synthesis of 2-(5-chloro-2-thienyl)propan-1-ol (S8) and (S9)

The racemic compound, 2-(5-chloro-2-thienyl)propan-1-ol C20 (12 g, 62.492 mmol) was separated into constituent enantiomers by chiral SFC separation. Column: Daicel Chiralpak® AD-H, 30×250 mm; Mobile Phase: 10% Methanol/Hexane Mixture (7:3), 90% carbon dioxide. Flow: 90g/min. 2-(5-chloro-2-thienyl)propan-1-ol S8 (4 g, 35%). ¹H NMR (Chloroform-d, 400 MHz) δ 6.76-6.75 (d, J=3.6 Hz, 1H), 6.66-6.65 (dd, J=3.6 Hz, 1H), 3.73-3.61 (m, 2H), 3.17-3.10 (m, 1H), 1.52-1.49 (t, J=5.2 Hz, 1H), 1.32-1.30 (d, J=6.8 Hz, 3H). GCMS: m z: 176.0 [M]⁺.

And 2-(5-chloro-2-thienyl)propan-1-ol S9 (3.75 g, 34%). ¹H NMR (Chloroform-d, 400 MHz) δ 6.76-6.75 (d, J=4 Hz, 1H), 6.66-6.65 (dd, J=3.6 Hz, 1H), 3.73-3.61 (m, 2H), 3.15-3.10 (q, J=6.8 Hz, 1H),1.51-1.48 (t, J=5.6 Hz, 1H), 1.33-1.30 (d, J=7.2 Hz, 3H). GCMS m/z 176.0 [M]⁺.

### Preparation S10 and S11

### 2-(5-ethyl-2-thienyl)propan-1-ol (S10 ENANT-1) and (S11 ENANT-2)

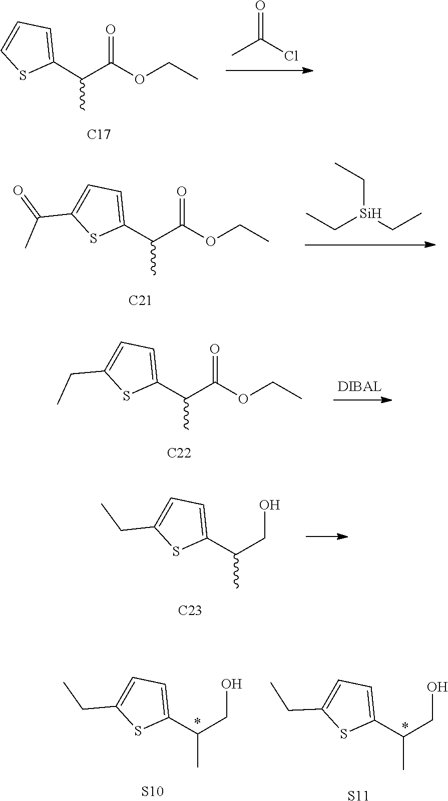

### Step 1. Synthesis of ethyl 2-(5-acetyl-2-thienyl)propanoate (C21)

To a stirred solution of ethyl 2-(2-thienyl)propanoate C17 (80 g, 336.92 mmol) in DCM (1500 mL) was added Acetyl chloride (39.671 g, 35.934 mL, 505.38 mmol) drop-wise at 0° C., followed by addition of AlCl₃ (67.388 g, 505.38 mmol) at 0° C. The reaction mixture was stirred for 2 hours at 0° C. The mixture was slowly quenched with ice water (1000 mL), the two layers were separated, and the aqueous layer was extracted with DCM (2×500 mL). The combined organic layers were washed with brine (500 mL), dried over sodium sulfate. Purification by silica gel chromatography (Gradient: 0-5% EtOAc in Petroleum ether) yielded the product ethyl 2-(5-acetyl-2-thienyl)propanoate C21 (60 g, 73%). ¹H NMR (Chloroform-d, 400 MHz) δ 7.56-7.54 (t, J=4.0 Hz, 1H), 6.99-6.98 (m, 1H), 4.20-4.14 (m, 2H), 4.01-3.96 (q, J=7.2 Hz, 1H), 2.52 (s, 3H), 1.60-1.56 (d, J=7.2 Hz, 3H), 1.28-1.23 (m, 3H). LCMS m/z 227.1 [M+H]⁺.

### Step 2. Synthesis of ethyl 2-(5-ethyl-2-thienyl)propanoate (C22)

To a stirred solution of ethyl 2-(5-acetyl-2-thienyl)propanoate C21 (60 g, 245.79 mmol) in TFA (400 mL) was added Triethylsilane (42.870 g, 58.9 mL, 368.69 mmol) drop-wise at 0° C. The reaction mixture was stirred for 4 hours at room temperature. The reaction was concentrated and quenched with ice water (500 mL) and extracted with EtOAc (3×500 mL). The combined organic layers were washed with brine (250 mL), dried over sodium sulfate, and concentrated to afford crude product. Purification by silica gel chromatography (Gradient: 0-3% EtOAc in Petroleum ether) yielded the product ethyl 2-(5-ethyl-2-thienyl)propanoate C22 (50 g, 82%). ¹H NMR (Chloroform-d, 400 MHz) δ 6.73-6.72 (dd, J=3.6 Hz, 1H), 6.62-6.60 (m, 1H), 4.18-4.13 (m, 2H), 3.93-3.88 (q, J=7.2 Hz, 1H), 2.82-2.78 (m, 2H), 1.55-1.53 (d, J=7.2 Hz, 3H) 1.30-1.23 (m, 6H). LCMS m/z 213.2 [M+H]⁺.

### Step 3. Synthesis of 2-(5-ethyl-2-thienyl)propan-1-ol (C23)

To a stirred solution of ethyl 2-(5-ethyl-2-thienyl)propanoate C22 (50 g, 200.18 mmol) in THF (1000 mL) was added DIBAL-H (25% in Toluene) (227.75 mL of 25% w/v, 400.36 mmol) drop-wise at 0° C. The reaction mixture was stirred for 2 hours at 0° C. The mixture was slowly quenched with saturated NH₄Cl solution (500 mL) at 0° C. and extracted with EtOAc (2×500 mL). The combined organic layers were washed with brine (250 mL), dried over sodium sulfate and concentrated. Purification by silica gel chromatography (Gradient: 0-5% EtOAc in Petroleum ether) yielded the product, 2-(5-ethyl-2-thienyl)propan-1-ol C23 (31 g, 89%). ¹H NMR (Chloroform-d, 400 MHz): δ 6.69-6.68 (d, J=3.6 Hz, 1H), 6.64-6.62 (m, 1H), 3.72-3.60 (m, 2H), 3.18-3.13 (q, J=6.8 Hz, 1H), 2.83-2.77 (m, 2H), 1.61-1.5 (m, 1H), 1.35-1.28 (m, 6H). LCMS m/z 171.02 [M+H]⁺.

### Step 4. Synthesis of 2-(5-ethyl-2-thienyl)propan-1-ol (S10) and (S11)

The racemic compound, 2-(5-ethyl-2-thienyl)propan-1-ol C23 (31 g, 178.06 mmol) was separated into constituent enantiomers by chiral SFC separation. Column: Daicel Chiralpak® AD-H, 30×250 mm; Mobile Phase: 10% Methanol/Hexane Mixture (7:3), 85% carbon dioxide. 2-(5-ethyl-2-thienyl)propan-1-ol S10 (13.45 g, 43%). ¹H NMR (Chloroform-d, 400 MHz): δ=6.69-6.68 (d, J=3.2 Hz, 1H), 6.63-6.62 (d, J=3.2 Hz, 1H), 3.73-3.61 (m, 2H), 3.19-3.14 (q, J=6.8 Hz, 1H), 2.83-2.78 (m, 2H), 1.54-1.47 (m, 1H), 1.35-1.27 (m, 6H). LCMS m/z 171.1 [M+H]⁺.

And 2-(5-ethyl-2-thienyl)propan-1-ol S11 (11.35 g, 37%). ¹H NMR (Chloroform-d, 400 MHz): δ 6.68-6.67 (d, J=3.6 Hz, 1H), 6.63 (d, J=3.6 Hz, 1H), 3.73-3.61 (m, 2H), 3.20-3.12 (m, 1H), 2.83-2.77 (q, J=7.6 Hz, 2H), 1.54-1.45 (m, 1H), 1.33-1.27 (m, 6H). LCMS m/z 171.1 [M+H]⁺.

### Preparation S12

### 2-(5-chloro-2-thienyl)ethanol (S12)

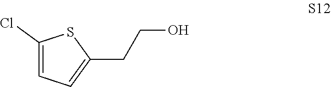

2-(5-chloro-2-thienyl)ethanol (S12) was obtained from commercial sources.

### Preparation S13

### 1-(2-methylsulfonylethyl)pyrazole-4-carbaldehyde (S13)

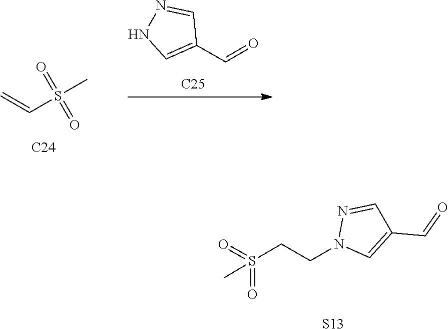

### Preparation of 1-(2-methylsulfonylethyl)pyrazole-4-carbaldehyde (S13)

A solution of 1H-pyrazole-4-carbaldehyde C25 (10 g, 104.1 mmol) 11-methylsulfonylethylene C24 (10 mL, 114.2 mmol) and K₂CO₃ (25 g, 180.9 mmol) in THF (200 mL) was stirred at 60° C. After stirring overnight, the mixture was cooled to room temperature and concentrated to dryness. The product was suspended in diethyl ether (100 mL) to triturate the product and stirred for 2 h. The product was filtered and dried overnight to yield 11-(2-methylsulfonylethyl)pyrazole-4-carbaldehyde S13 (20280 mg, 83%). ¹H NMR (400 MHz, DMSO-d₆) δ 9.80 (s, 1H), 8.54 (d, J=0.7 Hz, 1H), 8.05 (d, J=0.7 Hz, 1H), 4.64 (t, J=6.8 Hz, 2H), 3.80-3.67 (m, 2H), 2.96 (d, J=0.7 Hz, 3H). LCMS m/z 203.01 [M+H]⁺.

### Preparation S14

### 1-[2-[tert-butyl(dimethyl)silyl]oxyethyl]pyrazole-4-carbaldehyde (S20)

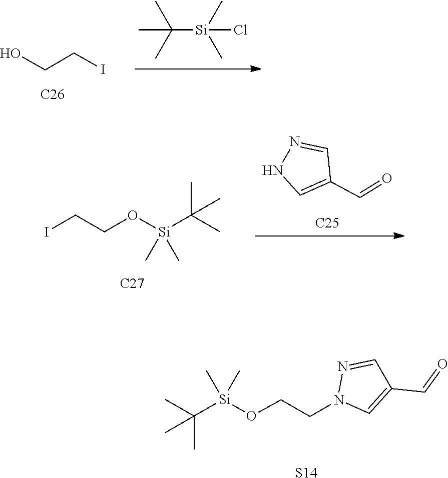

### Step 1. Synthesis of tert-butyl-(2-iodoethoxy)-dimethyl-silane (C27)

To a stirred solution of 2-iodoethanol C26 (2 g, 0.0116 mol) and Imidazole (1.58 g, 0.0232 mol) in DCM (40 mL) was added tert-butyl-chloro-dimethyl-silane (1.9 g, 0.0126 mol) at 0° C. Reaction was warmed to room temperature and stirred for 4 hours. The reaction mixture was diluted with DCM (100 mL), washed with sat. NaHCO₃ and brine, dried over Na₂SO₄ and concentrated under reduced pressure to get tert-butyl-(2-iodoethoxy)-dimethyl-silane C27 (2.5 g, 68%). ¹H NMR (400 MHz, Chloroform-d) δ 3.83 (t, J=6.8 Hz, 2H), 3.20 (t, J=6.8 Hz, 2H), 0.90 (s, 9H), 0.08 (s, 6H).

### Step 2. Synthesis of 1-[2-[tert-butyl(dimethyl)silyl]oxyethyl]pyrazole-4-carbaldehyde (S14)

To a solution of 1H-pyrazole-4-carbaldehyde C25 (20 g, 208.1 mmol) and K₂CO₃ (115 g, 832.1 mmol) in MeCN (200 mL) was added tert-butyl-(2-iodoethoxy)-dimethyl-silane C27 (65 g, 227.1 mmol). Reaction was heated to 80° C. Reaction was stirred for 5 hours. Reaction was cooled to 50° C. and stirred for 16 hours. Reaction mixture was warmed, filtered, and solids were washed with MeCN (200 mL). Solids were discarded. Filtrate was concentrated. Residue was partitioned between EtOAc (400 mL) and water (400 mL). The organic layer was separated, washed with water (400 mL) and brine (400 mL), dried over MgSO₄, filtered, and concentrated. Purification by silica gel chromatography (800 g column, 0-80% EtOAc in hexane) afforded the product 1-[2-[tert-butyl(dimethyl)silyl]oxyethyl]pyrazole-4-carbaldehyde S14 (46 g, 87%) as a pale yellow oil. ¹H NMR (300 MHz, Chloroform-d) δ 9.86 (s, 1H), 7.98 (s, 2H), 4.25 (dd, J=5.5, 4.5 Hz, 2H), 3.96 (dd, J=5.5, 4.5 Hz, 2H), 0.83 (s, 9H), −0.06 (s, 6H). LCMS m/z 255.14 [M+H]⁺.

### Preparation S15

### 1-methyltriazole-4-carbaldehyde (S15)

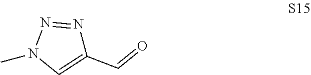

1-methyltriazole-4-carbaldehyde (S15) was obtained from commercial sources.

### Preparation S16

### tert-butyl (S)-2-ethynyl-4-oxopiperidine-1-carboxylate (S16)

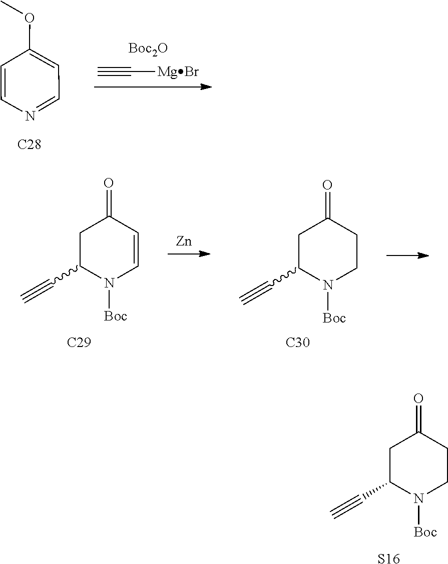

### Step 1. Synthesis of tert-butyl 2-ethynyl-4-oxo-2,3-dihydropyridine-1-carboxylate (C29)

To a solution of 4-methoxypyridine C28 (30.00 g, 274.91 mmol, 27.78 mL, 1.0 eq.) and Boc₂O (66.00 g, 302.40 mmol, 69.47 mL, 1.1 eq.) in THF (500 mL) was added ethynylmagnesium bromide (0.5 M, 825 mL, 1.5 eq.) dropwise at 0° C. The reaction was stirred at 25° C. for 3 hours. TLC (petroleum ether:Ethyl acetate=5:1) showed material A was consumed and the reaction was quenched by HCl aqueous (1.5 L, 1 M) under 0° C. bath. The reaction mixture was stirred at 25° C. for 0.5 hour and then extracted with ethyl acetate (500 mL×3). The organic layer was washed with brine (1 L×2), dried over Na₂SO₄, filtered, and concentrated in vacuum to give the residue. Purification by silica gel chromatography (0-10% EtOAc in Petroleum ether) afforded the product tert-butyl 2-ethynyl-4-oxo-2,3-dihydropyridine-1-carboxylate C29 (38.00 g, 171.75 mmol, 62.47% yield) as yellow solid. ¹H NMR (400 MHz, Chloroform-d) δ 7.72 (br s, 1H), 5.40 (d, J=8.4 Hz, 1H), 5.31 (br s, 1H), 2.85 (dd, J₁=6.4 Hz, J₂=16.4 Hz, 1H), 2.61 (d, J=16.4 Hz, 1H), 2.28 (d, J=2.4 Hz, 1H), 1.56 (s, 9H).

### Step 2. Synthesis of tert-butyl 2-ethynyl-4-oxopiperidine-1-carboxylate (C30)

To a solution of tert-butyl 2-ethynyl-4-oxo-2,3-dihydropyridine-1-carboxylate C29 (38.00 g, 171.75 mmol, 1.0 eq.) in AcOH (400 mL) was added Zn powder (82.21 g, 1.26 mol, 7.3 eq.) in portions within 5 mins at 25° C. The reaction was stirred at 55° C. for 4 hours under N₂. TLC (petroleum ether:ethyl acetate=5:1, KMnO₄) showed a main new spot. The reaction was filtrated and the cake was washed carefully with ethyl acetate (500 mL×3). All of the filtrates were concentrated in vacuum to give the residue. The residue was poured into 600 mL of ice water and extracted with ethyl acetate (500 mL×3). The organic layer were combined, washed with a solution of saturated sodium bicarbonate (500 mL×3) and brine (500 mL×2), dried over anhydrous Na₂SO₄, filtered and concentrated in vacuum to give crude product. The crude product was purified by silica gel column chromatography (petroleum ether/ethyl acetate=100:1-20:1) to give tert-butyl 2-ethynyl-4-oxopiperidine-1-carboxylate C30 (30.00 g, 131.68 mmol, 76.67% yield) as white solid. ¹H NMR (400 MHz, Chloroform-d) δ 5.44 (br s, 1H), 4.23 (d, J=11.2 Hz, 1H), 3.57-3.49 (m, 1H), 2.70 (dd, J₁=6.8 Hz, J₂=14.4 Hz, 1H), 3.54-2.40 (m, 3H), 2.41 (d, J=2.4 Hz, 1H), 1.50 (s, 9H). LCMS m/z 168.2 [M−55]⁺.

### Step 3. Synthesis of tert-butyl (S)-2-ethynyl-4-oxopiperidine-1-carboxylate (S16)

The racemic compound, tert-butyl 2-ethynyl-4-oxopiperidine-1-carboxylate C30 (50 g, 223.9 mmol) was separated into constituent enantiomers by chiral SFC separation. Column: Daicel Chiralpak® AD-H, 20×250 mm; Mobile Phase: 40% Methanol with 5 mM Ammonia, 60% carbon dioxide. The second-eluting peak was concentrated in vacuo to afford tert-butyl (S)-2-ethynyl-4-oxopiperidine-1-carboxylate S16 (22.5 g, 89%). ¹H NMR (Chloroform-d, 300 MHz): δ 5.45 (s, 1H), 4.24 (d, J=13.3 Hz, 1H), 3.54 (dt, J=13.3, 8.3 Hz, 1H), 2.93-2.24 (m, 5H), 1.51 (s, 9H). Note: The stereochemistry of S16 was confirmed by synthesizing compound 2 an alternative way. The data was convergent.

### Preparation S17

### 1-[(2,4-dimethoxyphenyl)methyl]-2-(1-methyltriazol-4-yl)piperidin-4-one (S17)

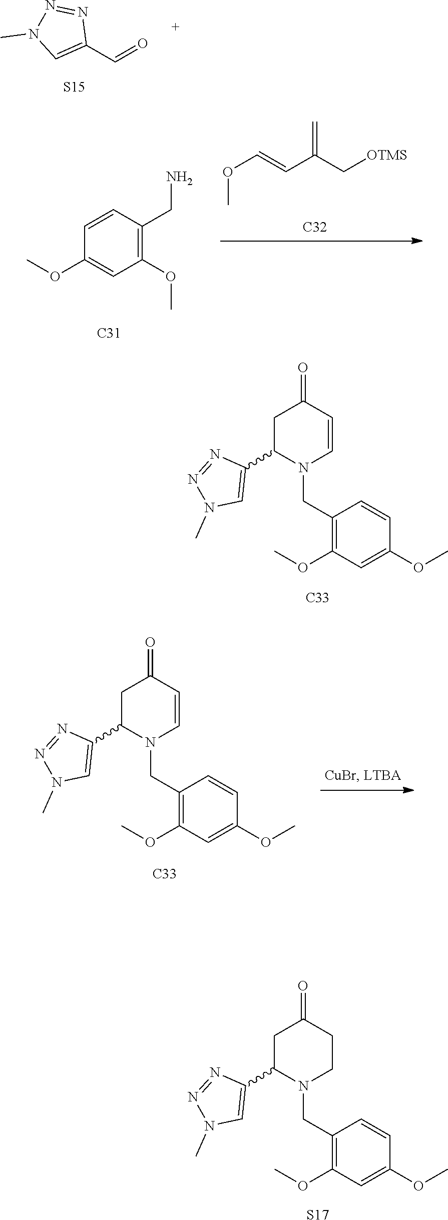

### Step 1. Synthesis of 1-[(2,4-dimethoxyphenyl)methyl]-2-(1-methyltriazol-4-yl)-2,3-dihydropyridin-4-one (C33)

1-methyltriazole-4-carbaldehyde S15 (3.1 g, 27.90 mmol) in MeOH (60 mL) was treated with (2,4-dimethoxyphenyl)methanamine C31 (4.5 mL, 29.95 mmol) and stirred at room temperature until ¹H NMR showed complete imine formation. [(Z)-3-methoxy-1-methylene-allyloxy]-trimethyl-silane C32 (10.5 mL, 53.93 mmol) was then added, and the reaction was stirred for 1 hour at room temperature. The reaction was quenched with 1 M HCl (8 mL) and stirred for 10 min. The mixture was then basified with sat. sodium bicarbonate and extracted with DCM (4×150 mL). The organic layers were dried over anhydrous Na₂SO₄, filtered and concentrated in vacuo to give crude product. The crude product was purified by silica gel column chromatography (0-60% of 20% MeOH/DCM in EtOAc) to afford 1-[(2,4-dimethoxyphenyl)methyl]-2-(1-methyltriazol-4-yl)-2,3-dihydropyridin-4-one C33 (5.66 g, 58%) as a yellow solid. ¹H NMR (400 MHz, Chloroform-d) δ 7.54 (s, 1H), 7.24 (dd, J=7.7, 0.8 Hz, 1H), 7.09 (dd, J=7.6, 0.9 Hz, 1H), 6.50 (d, J=7.7 Hz, 2H), 4.96 (dd, J=7.5, 1.1 Hz, 1H), 4.93-4.88 (m, 1H), 4.59-4.35 (m, 2H), 4.08 (s, 3H), 3.85 (s, 6H), 2.96 (dd, J=16.3, 7.4 Hz, 1H), 2.51 (ddd, J=16.3, 2.5, 1.2 Hz, 1H). LCMS m/z 329.1 [M+H]⁺.

### Step 2. Synthesis of 1-[(2,4-dimethoxyphenyl)methyl]-2-(1-methyltriazol-4-yl)piperidin-4-one (S17)

CuBr (360 mg, 2.510 mmol) in THF (35 mL) was cooled to −10° C. and lithium tri-tert-butoxyaluminum hydride (26 mL of 1 M in THF, 26.00 mmol) was added slowly. The mixture was stirred at −10° C. for 45 minutes and became a dark brown solution. This solution was then added slowly to a solution of 1-[(2,4-dimethoxyphenyl)methyl]-2-(1-methyltriazol-4-yl)-2,3-dihydropyridin-4-one C33 (5.66 g, 16.28 mmol) in THF (30 mL) which had been cooled to 0° C. The reaction was stirred at 0° C. for one hour and then was quenched with citric acid (16 mL of 2 M aqueous solution, 32.00 mmol), basified with 2 M NaOH until the pH reached 10, and diluted with DCM (150 mL). The organics were separated and the aqueous solution was extracted again with DCM (3×100 mL). The combined organic layers were dried over Na₂SO₄, filtered and concentrated in vacuo. The material was purified by silica gel column chromatography (isocratic EtOAc) to afford 1-[(2,4-dimethoxyphenyl)methyl]-2-(1-methyltriazol-4-yl)piperidin-4-one S17 (5.0 g, 87%) as a thick yellow oil. ¹H NMR (400 MHz, Chloroform-d) δ 7.42 (s, 1H), 7.32 (d, J=8.3 Hz, 1H), 6.48 (dd, J=8.3, 2.4 Hz, 1H), 6.44 (d, J=2.4 Hz, 1H), 4.35 (t, J=5.9 Hz, 1H), 4.09 (s, 3H), 3.80 (s, 3H), 3.77 (s, 3H), 3.63-3.53 (m, 2H), 2.95 (dt, J=12.2, 6.0 Hz, 1H), 2.84 (ddt, J=14.5, 5.4, 0.9 Hz, 1H), 2.74-2.66 (m, 2H), 2.50 (t, J=6.1 Hz, 2H). LCMS m/z 331.13 [M+H]⁺.

### Intermediates S18-S29

Intermediates S18-S29 (see Table 2) were prepared in two steps using the appropriate aldehyde and a similar method as described above for intermediate S17. Aldehydes were prepared by methods described above or obtained from commercial sources.

TABLE 2
Structure and physicochemical data for intermediates S18-S29

| | | Aldehyde | |
|---|---|---|---|
| Compound | Product | Reagent | ¹H NMR; LCMS m/z [M + H]⁺ |
| S18 | 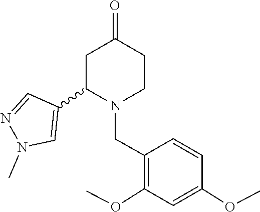 [SMILES: COc1ccc(CN2CCC(=O)CC2c2cnn(C)c2)c(OC)c1] | 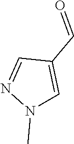 | LCMS m/z 330.09 [M + H]⁺ |
| S19 | 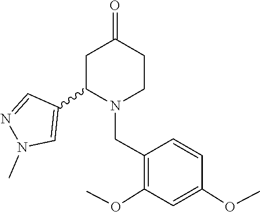 [SMILES: COc1ccc(CN2CCC(=O)CC2c2cnn(C)c2)c(OC)c1] | 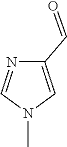 | LCMS m/z 330.14 [M + H]⁺ |
| S20 | 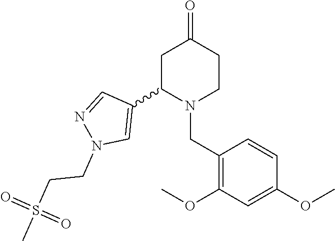 [SMILES: COc1ccc(CN2CCC(=O)CC2c2cnn(CCS(C)(=O)=O)c2)c(OC)c1] | 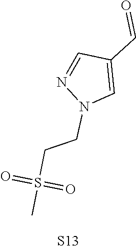 | ¹H NMR (400 MHz, Chloroform-d) δ 7.57 (s, 1H), 7.48 (s, 1H), 7.31 (d, J = 8.3 Hz, 1H), 6.51 (dd, J = 8.3, 2.4 Hz, 1H), 6.47 (d, J = 2.4 Hz, 1H), 4.66-4.57 (m, 2H), 3.99 (dd, J = 7.5, 4.9 Hz, 1H), 3.84 (s, 3H), 3.81 (s, 3H), 3.72-3.60 (m, 3H), 3.39 (d, J = 13.7 Hz, 1H), 3.03 (dt, J = 11.7, 5.6 Hz, 1H), 2.81-2.56 (m, 3H), 2.56-2.49 (m, 2H), 2.48 (s, 3H). LCMS m/z 422.26 [M + H]⁺ |
| S21 | 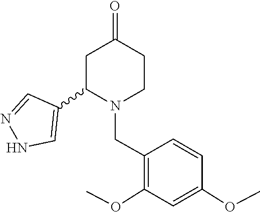 [SMILES: COc1ccc(CN2CCC(=O)CC2c2cn[nH]c2)c(OC)c1] | 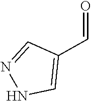 | ¹H NMR (400 MHz, Chloroform-d) δ 10.31 (s, 1H), 7.60 (s, 2H), 7.32 (d, J = 8.3 Hz, 1H), 6.51 (dd, J = 8.3, 2.4 Hz, 1H), 6.47 (d, J = 2.4 Hz, 1H), 4.05 (dd, J = 7.6, 4.9 Hz, 1H), 3.83 (s, 3H), 3.80 (s, 3H), 3.57 (dd, J = 118.3, 13.7 Hz, 2H), 3.10-3.01 (m, 1H), 2.81-2.43 (m, 5H). LCMS m/z 316.09 [M + H]⁺ |
| S22 | 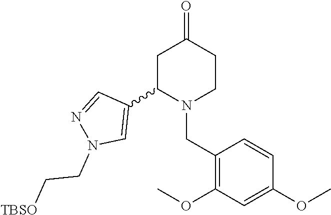 [SMILES: COc1ccc(CN2CCC(=O)CC2c2cnn(CCO[Si](C)(C)C(C)(C)C)c2)c(OC)c1] | 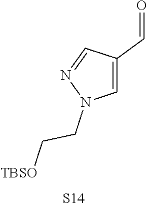 [SMILES: CC(C)(C)[Si](C)(C)OCCn1cc(C=O)cn1 ; "S14"] | ¹H NMR (400 MHz, Chloroform-d) δ 7.48 (d, J = 0.8 Hz, 1H), 7.35 (d, J = 0.8 Hz, 1H), 7.28 (d, J = 8.3 Hz, 1H), 6.49-6.39 (m, 2H), 4.16 (dd, J = 5.6, 4.7 Hz, 2H), 3.96-3.88 (m, 3H), 3.77 (d, J = 11.8 Hz, 6H), 3.71-3.65 (m, 1H), 3.35 (d, J = 13.7 Hz, 1H), 3.07-2.96 (m, 1H), 2.71-2.33 (m, 5H), 0.80 (s, 9H), −0.10 (s, 6H). LCMS m/z 474.42 [M + H]⁺ |
| S23 | 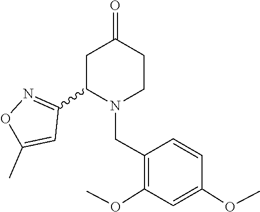 [SMILES: COc1ccc(CN2CCC(=O)CC2c2cc(C)on2)c(OC)c1] | 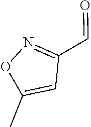 | ¹H NMR (300 MHz, Chloroform-d) δ 7.30 (d, J = 8.2 Hz, 1H), 6.56-6.41 (m, 2H), 6.10-6.02 (m, 1H), 4.13 (dd, J = 7.4, 5.7 Hz, 1H), 3.82 (s, 3H), 3.79 (s, 3H), 3.67 (d, J = 13.8 Hz, 1H), 3.46 (d, J = 13.8 Hz, 1H), 3.21-3.05 (m, 1H), 2.75-2.46 (m, 5H), 2.44 (d, J = 0.9 Hz, 3H). LCMS m/z 331.13 [M + H]⁺ |
| S24 | 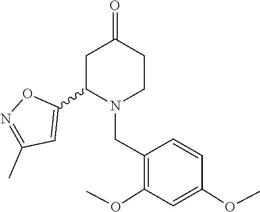 [SMILES: COc1ccc(CN2CCC(=O)CC2c2cc(C)no2)c(OC)c1] | 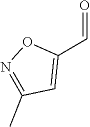 | ¹H NMR (400 MHz, Chloroform-d) δ 7.31 (d, J = 8.2 Hz, 1H), 6.49 (dd, J = 8.3, 2.4 Hz, 1H), 6.45 (d, J = 2.4 Hz, 1H), 5.98 (s, 1H), 4.32 (t, J = 5.5 Hz, 1H), 3.80 (d, J = 8.8 Hz, 6H), 3.65 (d, J = 1.5 Hz, 2H), 2.93-2.73 (m, 3H), 2.66 (ddd, J = 14.8, 5.4, 1.7 Hz, 1H), 2.59-2.39 (m, 2H), 2.30 (s, 3H). |
| S25 | 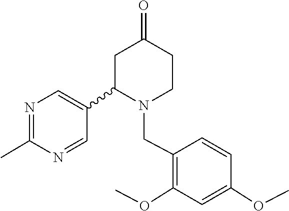 | 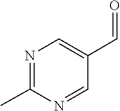 | ¹H NMR (400 MHz, Chloroform-d) δ 8.73 (s, 2H), 7.18 (d, J = 8.2 Hz, 1H), 6.51-6.39 (m, 2H), 3.82 (s, 3H), 3.78 (s, 3H), 3.71-3.62 (m, 2H), 3.39-3.29 (m, 1H), 3.12 (d, J = 13.3 Hz, 1H), 2.77 (s, 3H), 2.75-2.59 (m, 2H), 2.59-2.36 (m, 3H). LCMS m/z 342.25 [M + H]⁺ |
| S26 | 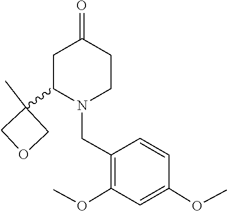 [SMILES: COc1ccc(CN2CCC(=O)CC2C2(C)COC2)c(OC)c1] | 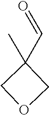 | LCMS m/z 320.18 [M + H]⁺ |
| S27 | 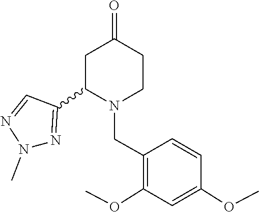 [SMILES: COc1ccc(CN2CCC(=O)CC2c2cnn(C)n2)c(OC)c1] | 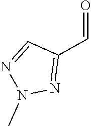 | LCMS m/z 331.35 [M + H]⁺ |
| S28 | 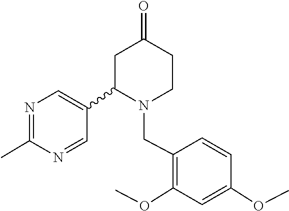 | 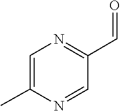 | ¹H NMR (400 MHz, Chloroform-d) δ 8.60 (d, J = 1.5 Hz, 1H), 8.46 (dd, J = 1.4, 0.7 Hz, 1H), 7.26 (d, J = 8.3 Hz, 1H), 6.49 (dd, J = 8.3, 2.4 Hz, 1H), 6.45 (d, J = 2.4 Hz, 1H), 4.06 (dd, J = 8.2, 4.8 Hz, 1H), 3.83 (s, 3H), 3.78 (s, 3H), 3.62-3.37 (m, 2H), 3.25-3.12 (m, 1H), 2.83-2.71 (m, 1H), 2.71-2.61 (m, 3H), 2.59 (s, 3H), 2.55- 2.41 (m, 1H). LCMS m/z 342.16 [M + H]⁺ |
| S29 | 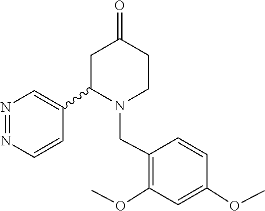 [SMILES: COc1ccc(CN2CCC(=O)CC2c2ccnnc2)c(OC)c1] | 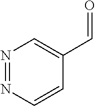 | ¹H NMR (400 MHz, Chloroform-d) δ 9.35 (dd, J = 2.4, 1.2 Hz, 1H), 9.16 (dd, J = 5.3, 1.2 Hz, 1H), 7.52 (dd, J = 5.3, 2.4 Hz, 1H), 7.17 (d, J = 8.3 Hz, 1H), 6.46 (dd, J = 8.3, 2.4 Hz, 1H), 6.42 (d, J = 2.4 Hz, 1H), 3.80 (s, 3H), 3.76 (s, 3H), 3.73 (d, J = 7.3 Hz, 1H), 3.61 (d, J = 13.2 Hz, 1H), 3.28 (ddd, J = 12.0, 5.6, 3.6 Hz, 1H), 3.18 (d, J = 13.3 Hz, 1H), 2.64 (ddd, J = 13.9, 11.5, 5.6 Hz, 1H), 2.56 (d, J = 7.5 Hz, 2H), 2.54-2.35 (m, 2H). |

### Preparation S30

### tert-butyl 4-oxo-2-phenyl-piperidine-1-carboxylate (S30)

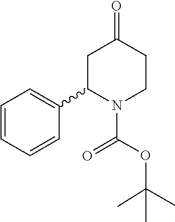 [SMILES: CC(C)(C)OC(=O)N1CCC(=O)CC1c1ccccc1]

Tert-butyl 4-oxo-2-phenyl-piperidine-1-carboxylate (S30) was obtained from commercial sources.

### Preparation S31

### benzyl 2-cyclopropyl-4-oxo-piperidine-1-carboxylate (S31)

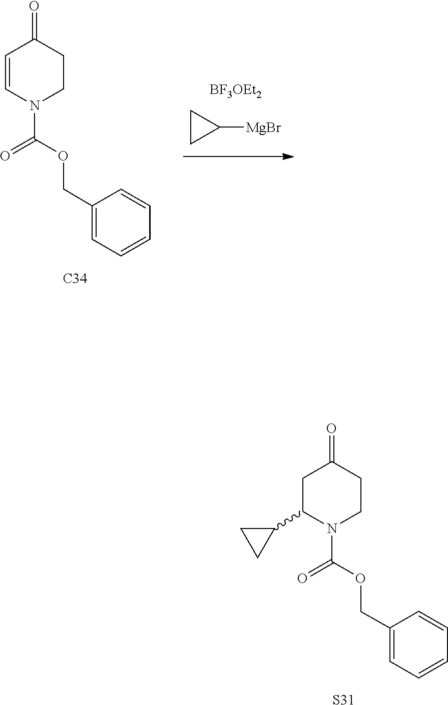

### Preparation of benzyl 2-cyclopropyl-4-oxo-piperidine-1-carboxylate (S31)

Under a nitrogen atmosphere, CuI (1.6 g, 8.401 mmol) in THF (11 mL) at −78° C. was treated with cyclopropylmagnesium bromide (18 mL of 0.5 M in THF, 9.00 mmol). The mixture was stirred at −78° C. for 30 min, then diethyloxonio(trifluoro)boranuide (801 μL, 6.490 mmol) was added. The mixture was stirred at −78° C. for another 10 min. Benzyl 4-oxo-2,3-dihydropyridine-1-carboxylate C34 (1 g, 4.324 mmol) in THF (4 mL) was then added and the mixture was continued stirring at −78° C. for two hours. The reaction was quenched with saturated NH₄Cl (3 mL) and the aqueous layer was separated and extracted with EtOAc (3×20 mL). The organic layers were dried over sodium sulfate and concentrated in vacuo. The material was purified by silica gel column chromatography (0-50% EtOAc in Heptane) to afford benzyl 2-cyclopropyl-4-oxo-piperidine-1-carboxylate S31 (463 mg, 36%). ¹H NMR (300 MHz, Chloroform-d) δ 7.36 (d, J=3.3 Hz, 5H), 5.16 (d, J=2.4 Hz, 2H), 4.42 (d, J=13.5 Hz, TH), 3.94 (s, TH), 3.46 (ddd, J=13.8, 11.8, 4.0 Hz, TH), 2.65 (dd, J=14.4, 6.7 Hz, TH), 2.57-2.31 (m, 3H), 0.91 (dddd, J=12.8, 9.7, 7.9, 4.8 Hz, TH), 0.69-0.54 (in, TH), 0.53-0.32 (m, 2H), 0.28 (dt, J=9.4, 4.8 Hz, TH). LCMS m/z 274.12 [M+H]⁺.

### Compound 1

### 2-chloro-2′-(2-methylpyrimidin-5-yl)spiro[4,5-dihydrothieno[2,3-c]pyran-7,4′-piperidine](1)

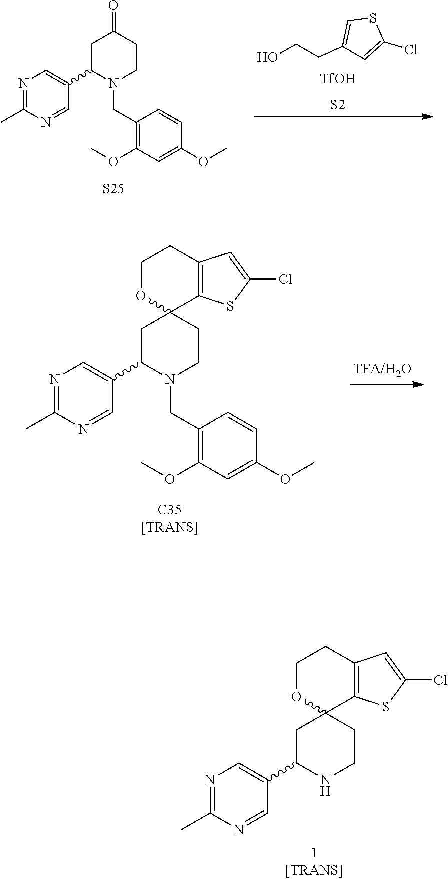

### Step 1. Synthesis of 2-chloro-1′-[(2,4-dimethoxyphenyl)methyl]-2′-(2-methylpyrimidin-5-yl)spiro[4,5-dihydrothieno[2,3-c]pyran-7,4′-piperidine](C35)

1-[(2,4-dimethoxyphenyl)methyl]-2-(2-methylpyrimidin-5-yl)piperidin-4-one S25 (120 mg, 0.3515 mmol) and 2-(5-chloro-3-thienyl)ethanol S2 (70 mg, 0.4046 mmol) were dissolved in dioxane (1 mL) and cooled in an ice bath and triflic acid (90 μL, 1.017 mmol) was added. The reaction mixture was allowed to warm to room temperature. After 3 hours the reaction mixture was diluted with 1 M NaOH and EtOAc (30 mL each) and the organic layer separated, dried over sodium sulfate and concentrated in vacuo. Purification by silica gel chromatography (Gradient: 0-10% MeOH in DCM) yielded the major product as an oil, 2-chloro-1′-[(2,4-dimethoxyphenyl)methyl]-2′-(2-methylpyrimidin-5-yl)spiro[4,5-dihydrothieno[2,3-c]pyran-7,4′-piperidine] C35 (83 mg, 49%)¹H NMR (400 MHz, Chloroform-d) δ 8.75 (s, 2H), 7.18 (d, J=8.2 Hz, 1H), 6.59 (s, 1H), 6.46 (dd, J=8.2, 2.4 Hz, 1H), 6.42 (d, J=2.4 Hz, 1H), 4.06-3.90 (m, 2H), 3.82 (s, 3H), 3.77 (s, 3H), 3.71-3.61 (m, 1H), 3.57 (d, J=13.2 Hz, 1H), 3.01 (d, J=13.1 Hz, 1H), 2.97-2.87 (m, 1H), 2.75 (s, 3H), 2.72-2.46 (m, 3H), 2.15-2.02 (m, 2H), 1.99-1.77 (m, 2H). LCMS m/z 486.26 [M+H]⁺. NMR was consistent with a pair of enantiomers. Assumed relative trans stereochemistry.

### Step 2. Synthesis of 2-chloro-2′-(2-methylpyrimidin-5-yl)spiro[4,5-dihydrothieno[2,3-c]pyran-7,4′-piperidine] (1)

2-chloro-1′-[(2,4-dimethoxyphenyl)methyl]-2′-(2-methylpyrimidin-5-yl)spiro[4,5-dihydrothieno[2,3-c]pyran-7,4′-piperidine] C35 (76 mg, 0.1564 mmol) was dissolved in TFA (1 mL) and H₂O (200 μL) and heated to 90° C. After 3 hours done by LCMS the reaction mixture was cooled to room temperature and diluted with 1 M NaOH and EtOAc (30 mL each). The organic layer was separated, dried over sodium sulfate, and concentrated to an oil. Purification by silica gel chromatography (Gradient: 0 to 10% MeOH in DCM) yielded the product 2-chloro-2′-(2-methylpyrimidin-5-yl)spiro[4,5-dihydrothieno[2,3-c]pyran-7,4′-piperidine]1 (34 mg, 63%) as a white solid. ¹H NMR (400 MHz, Chloroform-d) δ 8.69 (s, 2H), 6.61 (s, 1H), 4.15 (dd, J=11.6, 2.5 Hz, 1H), 4.05-3.91 (m, 2H), 3.32-3.19 (m, 1H), 3.14-3.01 (m, 1H), 2.75 (s, 3H), 2.72-2.56 (m, 2H), 2.20-2.04 (m, 2H), 1.91-1.70 (m, 2H). LCMS m/z 336.03 [M+H]⁺.

### Compound 2

### (2S,4S)-2′-chloro-2-(1-methyl-1H-1,2,3-triazol-4-yl)-4′,5′-dihydrospiro[piperidine-4,7′-thieno[2,3-c]pyran] (2 [ENANT-2])

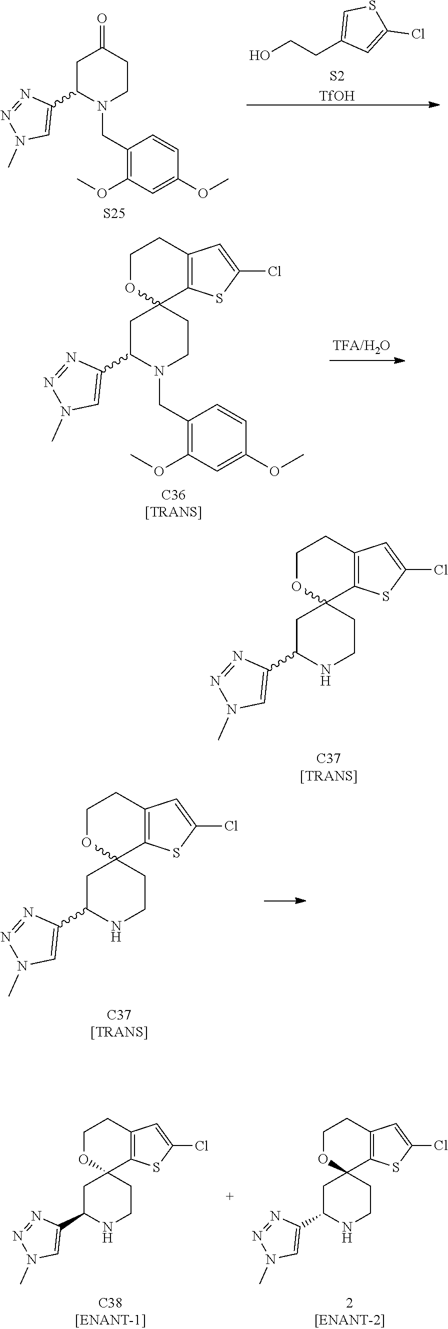

### Step 1. Synthesis of 2-chloro-1′-[(2,4-dimethoxyphenyl)methyl]-2′-(1-methyltriazol-4-yl)spiro[4,5-dihydrothieno[2,3-c]pyran-7,4′-piperidine](C36)

1-[(2,4-dimethoxyphenyl)methyl]-2-(1-methyltriazol-4-yl)piperidin-4-one S25 (3.5 g, 9.905 mmol) in DCM (50 mL) was cooled to 0° C. and treated with a solution of 2-(5-chloro-3-thienyl)ethanol S2 (1.82 g, 11.19 mmol) in DCM (10 mL). Triflic Acid (2.6 mL, 29.38 mmol) was added slowly, and the reaction was warmed to room temperature. After 30 min the reaction was carefully quenched with sat. sodium bicarbonate solution and the organics were separated via filtration through a phase separator. The organics were concentrated in vacuo and the material was purified via silica gel chromatography (Gradient: 20-100% EtOAc in Heptane). The first-eluting peak afforded 2-chloro-1′-[(2,4-dimethoxyphenyl)methyl]-2′-(1-methyltriazol-4-yl)spiro[4,5-dihydrothieno[2,3-c]pyran-7,4′-piperidine] C36 (3.1 g, 65%) as an off-white foam. ¹H NMR (400 MHz, Chloroform-d) δ 7.53 (s, 1H), 7.23 (d, J=8.3 Hz, 1H), 6.56 (s, 1H), 6.45 (dd, J=8.3, 2.4 Hz, 1H), 6.40 (d, J=2.4 Hz, 1H), 4.07 (s, 3H), 4.02 (dd, J=11.7, 2.9 Hz, 1H), 3.95 (dd, J=6.0, 4.9 Hz, 2H), 3.79 (s, 3H), 3.74 (s, 3H), 3.41 (dd, J=184.3, 13.8 Hz, 2H), 2.90-2.80 (m, 1H), 2.67-2.50 (m, 3H), 2.31 (dt, J=13.9, 2.8 Hz, 1H), 1.98-1.85 (m, 3H). LCMS m/z 475.32 [M+H]⁺.

### Step 2. Synthesis of 2-chloro-2′-(1-methyltriazol-4-yl)spiro[4,5-dihydrothieno[2,3-c]pyran-7,4′-piperidine] (C37)

In a large microwave vial, 2-chloro-1′-[(2,4-dimethoxyphenyl)methyl]-2′-(1-methyltriazol-4-yl)spiro[4,5-dihydrothieno[2,3-c]pyran-7,4′-piperidine] C36 (3.1 g, 6.416 mmol) in water (4 mL, 222.0 mmol) and TFA (14 mL, 181.7 mmol) was heated to 95° C. After 10 hours the reaction had turned a bright pink and LCMS showed consumption of starting material. The reaction was cooled to room temperature and the mixture was concentrated via rotovap to remove the volatiles. The remaining solution was diluted with DCM and quenched slowly with sat. sodium bicarbonate solution until the pink color subsided and the pH reached 9. The organics were separated, filtered through a pad of celite, and concentrated in vacuo. Purification by silica gel chromatography (Gradient: 0-12% MeOH in DCM) afforded 2-chloro-2′-(1-methyltriazol-4-yl)spiro[4,5-dihydrothieno[2,3-c]pyran-7,4′-piperidine] C37 (1.7 g, 80%) as a tan foam. ¹H NMR (400 MHz, Chloroform-d) δ 7.47 (s, 1H), 6.58 (s, 1H), 4.39 (dd, J=11.8, 2.6 Hz, 1H), 4.06 (s, 3H), 3.95 (t, J=5.5 Hz, 2H), 3.27 (td, J=12.5, 2.8 Hz, 1H), 3.05 (ddd, J=12.2, 4.7, 2.2 Hz, 1H), 2.61 (td, J=5.4, 3.0 Hz, 2H), 2.36 (dt, J=13.8, 2.7 Hz, 1H), 2.06 (dq, J=14.0, 2.6 Hz, 1H), 1.95-1.78 (m, 2H). LCMS m/z 325.1 [M+H]⁺.

### Step 3. Synthesis of (2R,4R)-2′-chloro-2-(1-methyl-1H-1,2,3-triazol-4-yl)-4′,5′-dihydrospiro[piperidine-4,7′-thieno[2,3-c]pyran] (C38 [ENANT-1]) and (2S,4S)-2′-chloro-2-(1-methyl-1H-1,2,3-triazol-4-yl)-4′,5′-dihydrospiro[piperidine-4,7′-thieno[2,3-c]pyran] (2 [ENANT-2])

2-chloro-2′-(1-methyltriazol-4-yl)spiro[4,5-dihydrothieno[2,3-c]pyran-7,4′-piperidine] C37 (1.09 g, 2.89 mmol) was separated into constituent enantiomers by chiral SFC separation. Column: Daicel Chiralpak® AD-H, 20×250 mm; Mobile Phase: 15% Methanol (5 mM ammonia), 85% carbon dioxide. Peak A was concentrated via rotovap to afford (2R,4R)-2′-chloro-2-(1-methyl-1H-1,2,3-triazol-4-yl)-4′,5′-dihydrospiro[piperidine-4,7′-thieno[2,3-c]pyran] C38 [ENANT-1](435 mg, 42%) as an off-white foam. ¹H NMR (400 MHz, Chloroform-d) δ 7.46 (s, 1H), 6.59 (s, 1H), 4.39 (dd, J=11.8, 2.7 Hz, 1H), 4.06 (s, 3H), 3.95 (t, J=5.5 Hz, 2H), 3.27 (td, J=12.5, 2.8 Hz, 1H), 3.05 (ddd, J=12.2, 4.7, 2.1 Hz, 1H), 2.61 (td, J=5.4, 3.2 Hz, 2H), 2.36 (dt, J=13.7, 2.7 Hz, 1H), 2.06 (dq, J=13.9, 2.6 Hz, 1H), 1.95-1.78 (m, 2H). LCMS m/z 325.14 [M+H]⁺.

Peak B was concentrated in vacuo to afford (2S,4S)-2′-chloro-2-(1-methyl-1H-1,2,3-triazol-4-yl)-4′,5′-dihydrospiro[piperidine-4,7′-thieno[2,3-c]pyran]2 [ENANT-2](455 mg, 45%) as a white solid. The structure and stereochemistry were confirmed via X-ray crystallography. ¹H NMR (400 MHz, Chloroform-d) δ 7.42 (s, 1H), 6.58 (s, 1H), 4.35 (dd, J=11.7, 2.6 Hz, 1H), 4.06 (s, 3H), 3.99-3.93 (m, 2H), 3.24 (td, J=12.4, 2.7 Hz, 1H), 3.02 (ddd, J=12.2, 4.8, 2.2 Hz, 1H), 2.61 (td, J=5.4, 3.8 Hz, 2H), 2.36 (dt, J=13.6, 2.7 Hz, 1H), 2.05 (dq, J=13.8, 2.5 Hz, 1H), 1.88-1.74 (m, 2H). LCMS m/z 325.14 [M+H]⁺.

### Compound 3

### 2′-cyclopropyl-2-ethyl-spiro[6,7-dihydrothieno[3,2-c]pyran-4,4′-piperidine] (3)

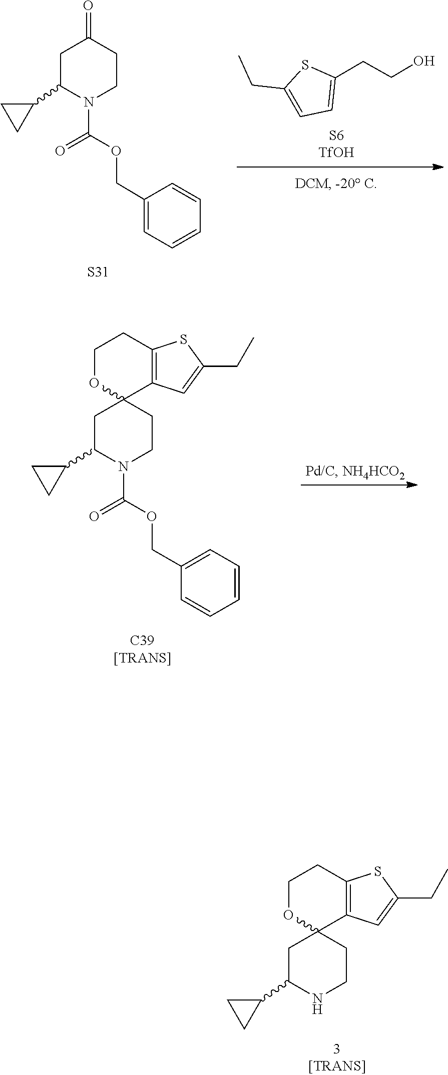

### Step 1. Synthesis of benzyl 2′-cyclopropyl-2-ethyl-spiro[6,7-dihydrothieno[3,2-c]pyran-4,4′-piperidine]-1′-carboxylate (C39)

Benzyl 2-cyclopropyl-4-oxo-piperidine-1-carboxylate S31 (57 mg, 0.1989 mmol) and 2-(5-ethyl-2-thienyl)ethanol S6 (40 mg, 0.2432 mmol) were dissolved in DCM (1 mL). The mixture was cooled to −20° C., to which trifluoromethanesulfonic acid (53 μL, 0.5989 mmol) was added. The mixture was stirred at −20° C. for 30 min and then quenched with saturated sodium bicarbonate solution. The mixture was extracted with DCM (2×3 mL). The combined organic layers were dried down and purification via silica gel chromatography (Gradient: 0-5% 7 M Ammonia in MeOH in DCM) afforded benzyl 2′-cyclopropyl-2-ethyl-spiro[6,7-dihydrothieno[3,2-c]pyran-4,4′-piperidine]-1′-carboxylate C39 (69 mg, 77%) as a pair of trans enantiomers. LCMS m/z 412.21 [M+H]⁺.

### Step 2. Synthesis of 2′-cyclopropyl-2-ethyl-spiro[6,7-dihydrothieno[3,2-c]pyran-4,4′-piperidine] (3)

In a microwave vial, benzyl 2′-cyclopropyl-2-ethyl-spiro[6,7-dihydrothieno[3,2-c]pyran-4,4′-piperidine]-1′-carboxylate C39 (69 mg, 0.153 mmol) was dissolved in MeOH (2 mL). Ammonium formate (89 mg, 1.411 mmol) was added, followed by Pd/C (22 mg of 10 wt %, 0.02067 mmol). The mixture was heated to 140° C. in the microwave for 10 min. The reaction mixture was filtered through a pad of Celite® and the filtrate concentrated in vacuo. Purification via silica gel chromatography (Gradient: 0-10 7 M Ammonia in MeOH in DCM) afforded 2′-cyclopropyl-2-ethyl-spiro[6,7-dihydrothieno[3,2-c]pyran-4,4′-piperidine]3 (24 mg, 38%). ¹H NMR (300 MHz, Chloroform-d) δ 6.50 (d, J=1.1 Hz, 1H), 4.04-3.78 (m, 2H), 3.12-2.86 (m, 2H), 2.86-2.63 (m, 4H), 2.18-1.92 (m, 2H), 1.91-1.79 (m, 2H), 1.65 (dd, J=13.6, 11.4 Hz, 1H), 1.28 (t, J=7.5 Hz, 3H), 0.81-0.62 (m, 1H), 0.57-0.29 (m, 2H), 0.24-0.06 (in, 2H). LCMS m/z 278.17 [M+H]⁺.

### Compounds 4-28

Compounds 4-28 (see Table 3) were prepared by methods similar to compounds 1, 2, or 3 with modifications obvious to someone skilled in the art. Thiophene ethanols and piperidones were prepared by methods described above or obtained from commercial sources.

TABLE 3
Method of preparation, structure and physicochemical data for compounds 4-28.

| | | Piperidone and | | ¹H NMR; LCMS m/z |
|---|---|---|---|---|
| Compound | Product | Thiophene ethanol | Method | [M + H]⁺ |
| 4 | 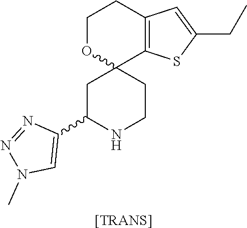 | 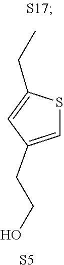 | Compound 1 | ¹H NMR (300 MHz, Chloroform-d) δ 7.41 (s, 1H), 6.43 (d, J = 1.3 Hz, 1H), 4.35 (dd, J = 11.8, 2.6 Hz, 1H), 4.04 (s, 3H), 3.94 (d, J = 5.5 Hz, 2H), 3.24 (td, J = 12.3, 2.8 Hz, 1H), 3.00 (ddd, J = 12.1, 4.8, 2.2 Hz, 1H), 2.76 (qd, J = 7.5, 1.0 Hz, 2H), 2.61 (td, J = 5.4, 2.2 Hz, 2H), 2.33 (dt, J = 13.7, 2.6 Hz, 1H), 2.03-1.97 (m, 1H), 1.95-1.77 (m, 2H), 1.25 (td, J = 7.3, 4.5 Hz, 3H). LCMS m/z 319.16 [M + H]⁺ |
| 5 | 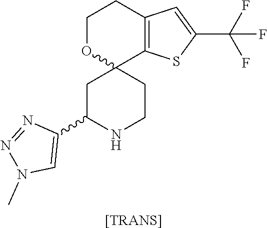 | 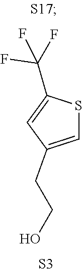 | Compound 1¹ | ¹H NMR (400 MHz, Methanol-d₄) δ 8.06 (d, J = 4.6 Hz, 1H), 7.36 (d, J = 1.3 Hz, 1H), 4.92-4.90 (m, 1H), 4.12 (s, 3H), 4.07 (td, J = 5.7, 3.4 Hz, 2H), 3.59 (td, J = 13.2, 3.2 Hz, 1H), 3.44 (ddd, J = 13.0, 4.7, 2.0 Hz, 1H), 2.79 (t, J = 5.5 Hz, 2H), 2.62 (dt, J = 14.7, 3.0 Hz, 1H), 2.47-2.37 (m, 2H), 2.16 (td, J = 14.0, 4.6 Hz, 1H). LCMS m/z 359.34 [M + H]⁺ |
| 6 | 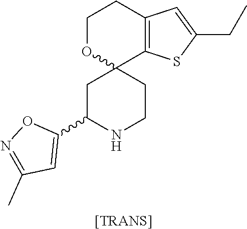 | 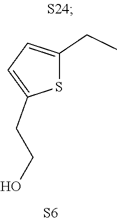 | Compound 1¹ | ¹H NMR (400 MHz, Methanol-d₄) δ 6.57 (d, J = 1.0 Hz, 1H), 6.44 (d, J = 11.2 Hz, 1H), 4.06-3.95 (m, 3H), 3.33 (t, J = 1.6 Hz, 1H), 2.79 (q, J = 7.6 Hz, 4H), 2.41-2.22 (m, 2H), 2.31 (d, J = 2.0 Hz, 3H), 2.16-2.02 (m, 2H), 1.27 (t, J = 7.6 Hz, 3H). Note: 1H obscured under MeOD peak. LCMS m/z 319.33 [M + H]⁺ |
| 7 | 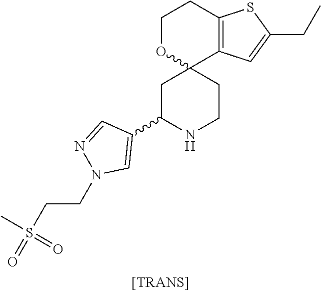 | 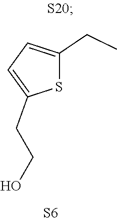 | Compound 1 | ¹H NMR (400 MHz, Chloroform-d) δ 7.58 (s, 1H), 7.50 (s, 1H), 6.54-6.45 (m, 1H), 4.63-4.51 (m, 2H), 4.13 (dd, J = 11.7, 2.6 Hz, 1H), 4.05-3.88 (m, 2H), 3.83-2.74 (m, 1H), 3.72-3.60 (m, 2H), 3.23 (td, J = 12.2, 3.1 Hz, 1H), 3.08-2.98 (m, 1H), 2.86-2.71 (m, 3H), 2.47-2.41 (m, 3H), 2.13-1.99 (m, 1H), 1.99-1.71 (m, 3H), 1.30 (t, J = 7.5 Hz, 3H). LCMS m/z 410.19 [M + H]⁺ |
| 8 | 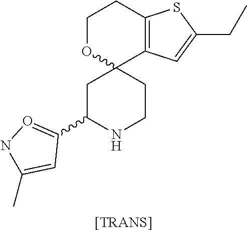 | 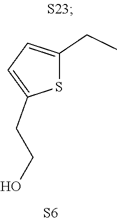 | Compound 1 | ¹H NMR (300 MHz, Chloroform-d) δ 6.48 (d, J = 1.1 Hz, 1H), 5.99 (d, J = 1.0 Hz, 1H), 4.30 (dd, J = 11.8, 2.7 Hz, 1H), 3.97 (t, J = 5.4 Hz, 2H), 3.97 (t, J = 5.4 Hz, 2H), 3.23 (ddd, J = 12.0, 9.0, 6.5 Hz, 1H), 3.10-2.96 (m, 1H), 2.88-2.67 (m, 4H), 2.40 (d, J = 0.8 Hz, 3H), 2.14 (dt, J = 13.8, 2.3 Hz, 1H), 1.89 (ddt, J = 8.8, 3.5, 1.7 Hz, 3H), 1.28 (t, J = 7.5 Hz, 3H). LCMS m/z 319.51 [M + H]⁺ |
| 9 | 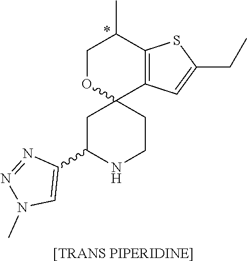 | 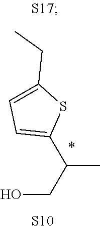 | Compound 1 | ¹H NMR (300 MHz, Chloroform-d) δ 7.49 (d, J = 6.1 Hz, 1H), 6.47 (p, J = 1.0 Hz, 1H), 4.47-4.35 (m, 1H), 4.04 (d, J = 1.6 Hz, 3H), 3.99-3.46 (m, 2H), 3.29 (dt, J = 12.3, 3.3 Hz, 1H), 3.11- 2.88 (m, 2H), 2.76 (q, J = 7.5 Hz, 2H), 2.30-2.05 (m, 2H), 1.99-1.78 (m, 2H), 1.24 (s, 6H). LCMS m/z 333.11 [M + H]⁺ |
| 10 | 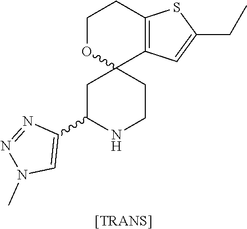 | 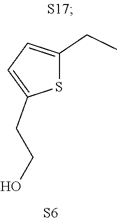 | Compound 1 | LCMS m/z 319.06 [M + H]⁺ |
| 11 | 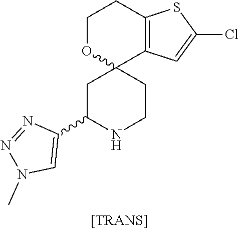 | 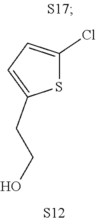 | Compound 1¹ | LCMS m/z 325.14 [M + H]⁺ |
| 12 | 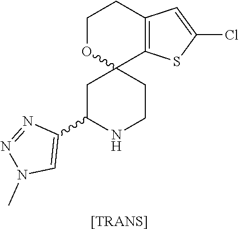 | 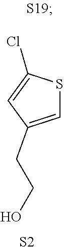 | Compound 1² | ¹H NMR (300 MHz, Acetonitrile-d₃) δ 8.50-8.36 (m, 1H), 7.50 (d, J = 1.5 Hz, 1H), 6.78 (s, 1H), 4.88 (dd, J = 12.7, 3.2 Hz, 1H), 4.02-3.89 (m, 2H), 3.82 (s, 3H), 3.41 (tdd, J = 9.6, 6.2, 3.7 Hz, 2H), 2.73-2.59 (m, 3H), 2.42 (ddd, J = 14.4, 3.2, 1.9 Hz, 1H), 2.30-2.09 (m, 2H). LCMS m/z 324.06 [M + H]⁺ |
| 13 | 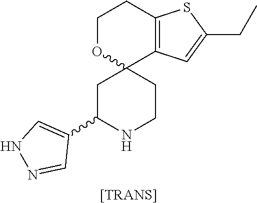 | 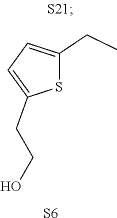 | Compound 1³ | ¹H NMR (400 MHz, Methanol-d₄) δ 8.31 (s, 2H), 7.80 (s, 2H), 6.63 (d, J = 1.0 Hz, 1H), 4.70 (dd, J = 12.5, 3.2 Hz, 1H), 3.99 (td, J = 5.5, 1.2 Hz, 2H), 3.51 (ddd, J = 12.7, 11.2, 5.6 Hz, 1H), 3.37-3.32 (m, 1H), 2.84-2.74 (m, 4H), 2.40 (dd, J = 14.7, 12.5 Hz, 1H), 2.33- 2.24 (m, 1H), 2.20-2.05 (m, 2H), 1.28 (t, J = 7.5 Hz, 3H). LCMS m/z 304.34 [M + H]⁺ |
| 14 | 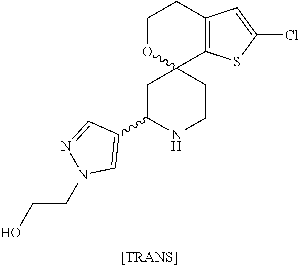 | 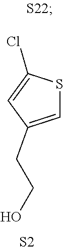 | Compound 1 | ¹H NMR (400 MHz, Chloroform-d) δ 7.48 (d, J = 0.8 Hz, 1H), 7.40 (t, J = 0.7 Hz, 1H), 6.58 (s, 1H), 4.23-4.16 (m, 2H), 4.10 (dd, J = 11.6, 2.6 Hz, 1H), 4.00-3.87 (m, 4H), 3.19 (td, J = 12.4, 2.7 Hz, 1H), 2.99 (ddd, J = 12.1, 4.8, 2.2 Hz, 1H), 2.61 (td, J = 5.5, 3.0 Hz, 2H), 2.23 (dt, J = 13.7, 2.6 Hz, 1H), 2.06 (dt, J = 14.0, 2.5 Hz, 1H), 1.82-1.67 (m, 2H). LCMS m/z 354.08 [M + H]⁺ |
| 15 | 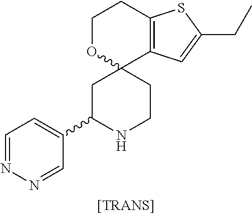 | 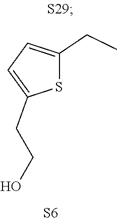 | Compound 1¹ | ¹H NMR (400 MHz, Chloroform-d) δ 10.71 (s, 1H), 10.08 (s, 1H), 9.87 (s, 1H), 9.54 (s, 1H), 8.68 (s, 1H), 6.64 (s, 1H), 4.91 (s, 1H), 3.98 (d, J = 16.1 Hz, 2H), 3.56 (s, 2H), 2.86-2.71 (m, 5H), 2.66 (t, J = 11.9 Hz, 1H), 2.14 (dd, J = 26.1, 14.5 Hz, 2H), 1.27 (d, J = 8.4 Hz, 3H). LCMS m/z 316.13 [M + H]⁺ |
| 16 | 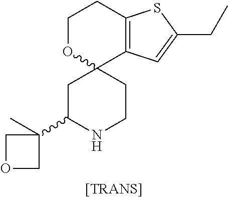 | 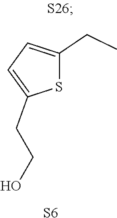 | Compound 1 | ¹H NMR (300 MHz, Chloroform-d) δ 6.47 (d, J = 1.0 Hz, 1H), 4.65 (d, J = 5.7 Hz, 1H), 4.52 (d, J = 5.9 Hz, 1H), 4.35 (d, J = 5.7 Hz, 1H), 4.26 (d, J = 5.9 Hz, 1H), 3.95 (t, J = 5.4 Hz, 2H), 3.36 (dd, J = 11.8, 2.3 Hz, 1H), 3.22- 2.96 (m, 2H), 2.84-2.71 (m, 4H), 1.89 (dq, J = 13.9, 2.6 Hz, 1H), 1.82- 1.72 (m, 2H), 1.48-1.37 (m, 1H), 1.34 (s, 3H), 1.30 (t, J = 7.5 Hz, 3H). LCMS m/z 308.16 [M + H]⁺ |
| 17 | 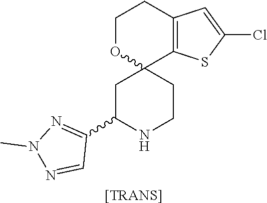 | 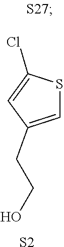 | Compound 1² | ¹H NMR (300 MHz, Acetonitrile-d₃) δ 7.74 (s, 1H), 6.79 (d, J = 1.1 Hz, 1H), 5.03-4.62 (m, 1H), 4.15 (d, J = 1.5 Hz, 3H), 3.98 (t, J = 5.5 Hz, 2H), 3.41 (ddd, J = 12.8, 4.9, 2.5 Hz, 2H), 2.66 (t, J = 5.5 Hz, 2H), 2.62-2.34 (m, 2H), 2.33-2.10 (m, 2H). LCMS m/z 325.1 [M + H]⁺ |
| 18 | 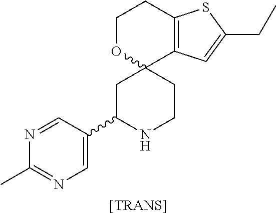 | 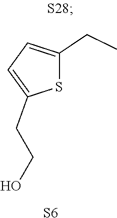 | Compound 1¹ | ¹H NMR (400 MHz, DMSO-d₆) δ 9.72 (d, J = 10.7 Hz, 1H), 9.41 (d, J = 10.2 Hz, 1H), 8.78 (d, J = 1.5 Hz, 1H), 8.63 (d, J = 1.4 Hz, 1H), 6.51 (d, J = 1.1 Hz, 1H), 4.68 (q, J = 8.6 Hz, 1H), 4.08-3.85 (m, 2H), 3.27 (d, J = 13.7 Hz, 2H), 2.84-2.61 (m, 4H), 2.54 (s, 3H), 2.34-2.20 (m, 3H), 2.00 (d, J = 14.6 Hz, 1H), 1.19 (t, J = 7.5 Hz, 3H). LCMS m/z 329.59 [M + H]⁺ |
| 19 | 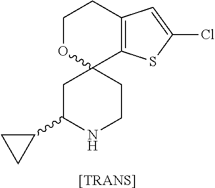 | 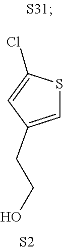 | Compound 3 | ¹H NMR (300 MHz, Chloroform-d) δ 6.60 (s, 1H), 3.95-3.74 (m, 2H), 3.35 (ddd, J = 23.5, 12.0, 4.8 Hz, 2H), 2.71- 2.52 (m, 3H), 2.31 (d, J = 14.6 Hz, 1H), 2.18-1.95 (m, 3H), 1.09 (dq, J = 12.7, 7.7, 5.7 Hz, 1H), 0.77-0.52 (m, 3H), 0.29 (d, J = 7.0 Hz, 1H). LCMS m/z 284.06 [M + H]⁺ |
| 20 | 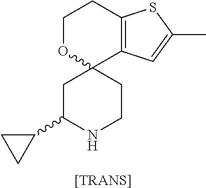 | 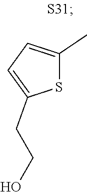 | Compound 3 | ¹H NMR (300 MHz, Chloroform-d) δ 6.46 (d, J = 1.3 Hz, 1H), 4.05-3.70 (m, 2H), 3.11-2.86 (m, 2H), 2.73 (q, J = 5.6 Hz, 2H), 2.42 (d, J = 1.1 Hz, 3H), 2.12-1.91 (m, 3H), 1.89-1.70 (m, 2H), 1.63 (dd, J = 13.6, 11.4 Hz, 1H), 0.84- 0.58 (m, 1H), 0.57-0.29 (m, 2H), 0.21 (s, 2H). LCMS m/z 264.12 [M + H]⁺ |
| 21 | 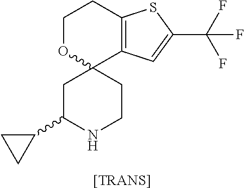 | 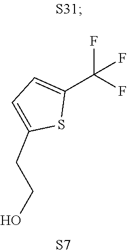 [SMILES: OCCc1ccc(C(F)(F)F)s1 ; "S7"] | Compound 3² | ¹H NMR (300 MHz, Acetonitrile-d₃) δ 7.52-7.40 (m, 1H), 7.30 (s, 1H), 4.13- 3.83 (m, 2H), 3.36-3.13 (m, 3H), 2.88 (td, J = 5.4, 1.4 Hz, 2H), 2.69 (q, J = 10.8, 9.9 Hz, 1H), 2.41-2.21 (m, 2H), 2.21-2.09 (m, 1H), 1.06 (dtt, J = 9.7, 7.9, 4.8 Hz, 1H), 0.64 (dtd, J = 8.2, 2.6, 1.3 Hz, 2H), 0.59-0.49 (m, 1H), 0.40-0.24 (m, 1H). LCMS m/z 318.43 [M + H]⁺ |
| 22 | 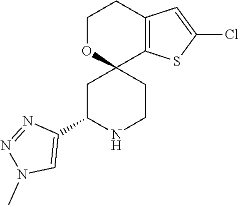 | 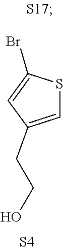 | Compound 1² | ¹H NMR (400 MHz, Methanol-d₄) δ 8.03 (s, 1H), 6.89 (s, 1H), 4.88-4.82 (m, 1H), 4.12 (s, 3H), 4.01 (td, J = 5.6, 2.7 Hz, 2H), 3.55 (td, J = 13.1, 3.2 Hz, 1H), 3.40 (ddd, J = 12.9, 4.7, 2.0 Hz, 1H), 2.70 (t, J = 5.5 Hz, 2H), 2.56 (dt, J = 14.6, 2.9 Hz, 1H), 2.39-2.30 (m, 2H), 2.08 (ddd, J = 14.9, 13.4, 4.7 Hz, 1H). LCMS m/z 369.23 [M + H]⁺ |
| 23 | 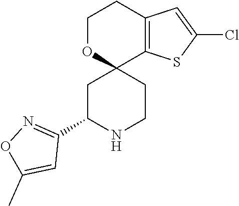 [SMILES: Cc1cc([C@@H]2C[C@]3(CCN2)OCCc2cc(Cl)sc23)no1] | 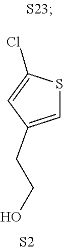 | Compound 2⁴ | ¹H NMR (400 MHz, Chloroform-d) δ 6.60 (s, 1H), 6.00 (s, 1H), 4.30 (dd, J = 11.7, 2.7 Hz, 1H), 3.96 (t, J = 5.5 Hz, 2H), 3.23 (td, J = 12.4, 2.7 Hz, 1H), 3.04 (ddd, J = 12.1, 4.7, 2.2 Hz, 1H), 2.63 (td, J = 5.4, 2.2 Hz, 2H), 2.41 (s, 3H), 2.30 (dt, J = 13.6, 2.7 Hz, 1H), 2.06 (dq, J = 13.9, 2.5 Hz, 1H), 1.88-1.72 (m, 3H). LCMS m/z 325.1 [M + H]⁺ |
| 24 | 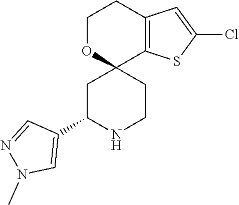 [SMILES: Cn1cc([C@@H]2C[C@]3(CCN2)OCCc2cc(Cl)sc23)cn1] | 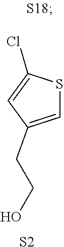 | Compound 2⁵ | LCMS m/z 324.01 [M + H]⁺ |
| 25 | 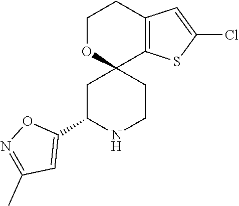 [SMILES: Cc1cc([C@@H]2C[C@]3(CCN2)OCCc2cc(Cl)sc23)on1] | 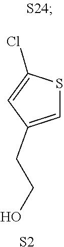 | Compound 2⁶ | ¹H NMR (400 MHz, Chloroform-d) δ 6.62 (s, 1H), 6.00 (s, 1H), 4.39-4.27 (m, 1H), 3.97 (td, J = 5.6, 1.2 Hz, 2H), 3.24 (td, J = 12.4, 2.7 Hz, 1H), 3.06 (ddd, J = 12.1, 4.8, 2.2 Hz, 1H), 2.65 (t, J = 5.5 Hz, 2H), 2.38 (dt, J = 13.6, 2.7 Hz, 1H), 2.30 (s, 3H), 2.08 (dq, J = 13.9, 2.5 Hz, 1H), 1.87-1.75 (m, 2H). LCMS m/z 325.05 [M + H]⁺ |
| 26 | 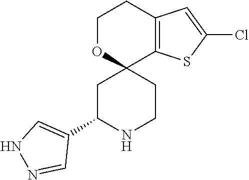 [SMILES: Clc1cc2c(s1)[C@@]1(CCN[C@H](c3cn[nH]c3)C1)OCC2] | 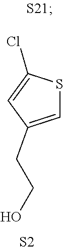 | Compound 2 | ¹H NMR (400 MHz, Chloroform-d) δ 7.59 (s, 2H), 6.61 (s, 1H), 4.19 (dd, J = 11.6, 2.6 Hz, 1H), 4.04-3.92 (m, 2H), 3.24 (td, J = 12.4, 2.7 Hz, 1H), 3.04 (ddd, J = 12.1, 4.8, 2.2 Hz, 1H), 2.72-2.58 (m, 2H), 2.27 (dt, J = 13.7, 2.7 Hz, 1H), 2.10 (dq, J = 13.9, 2.6 Hz, 1H), 1.86-1.73 (m, 2H). LCMS m/z 310.11 [M + H]⁺ |
| 27 | 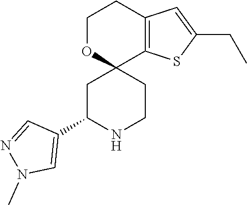 [SMILES: CCc1cc2c(s1)[C@@]1(CCN[C@H](c3cnn(C)c3)C1)OCC2] | 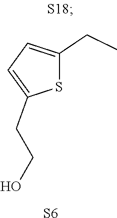 | Compound 2⁷ | LCMS m/z 318.11 [M + H]⁺ |
| 28 | 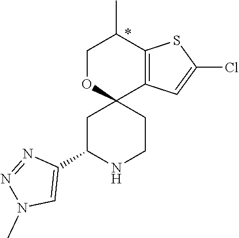 | 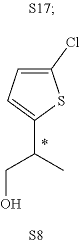 | Compound 2¹ | ¹H NMR (300 MHz, Methanol-d₄) δ 8.06 (s, 1H), 6.81 (s, 1H), 4.81 (dd, J = 3.4 Hz, 1H), 4.12 (s, 3H), 4.06 (dd, J = 11.5, 4.6 Hz, 1H), 3.61 (dd, J = 11.5, 7.1 Hz, 1H), 3.51 (dd, J = 12.8, 3.6 Hz, 1H), 3.44-3.34 (m, 1H), 3.02 (td, J = 7.0, 4.7 Hz, 1H), 2.44 (dd, J = 14.5, 12.4 Hz, 1H), 2.35-2.17 (m, 2H), 2.17-2.04 (m, 1H), 1.24 (d, J = 6.9 Hz, 3H). LCMS m/z 339.1 [M + H]⁺ |

Footnotes:
¹The product was isolated as the hydrochloride salt.
²The product was isolated as the trifluoroacetate salt.
³The product was isolated as the formate salt.
⁴The desired product was isolated as Peak B via chiral SFC purification: Chiralpak ® IC, 10 × 250 mm; Mobile Phase: 40% IPA (5 mM ammonia), 60%
carbon dioxide.
⁵The desired product was isolated as Peak A via chiral HPLC: Chiralpak ® AD-H, 20 × 250 mm; Mobile Phase: 50% Hexane, 50% IPA, 0.2% diethylamine.
⁶The desired product was isolated as Peak B via chiral SFC purification: Phenomenex Lux ® Cellulose-2, 20 × 250 mm; Mobile Phase: 20% MeOH (5 mM
ammonia), 80% carbon dioxide.
⁷The desired product was isolated as Peak B via chiral HPLC: Chiralpak ® OJ-H, 20 × 250 mm; Mobile Phase: 50% EtOH, 50% MeOH, 0.2% diethylamine.

### Compound 29

### 2′-cyclopropyl-2-ethyl-spiro[6,7-dihydrothieno[3,2-c]pyran-4,4′-piperidine] (29 [ENANT-1])

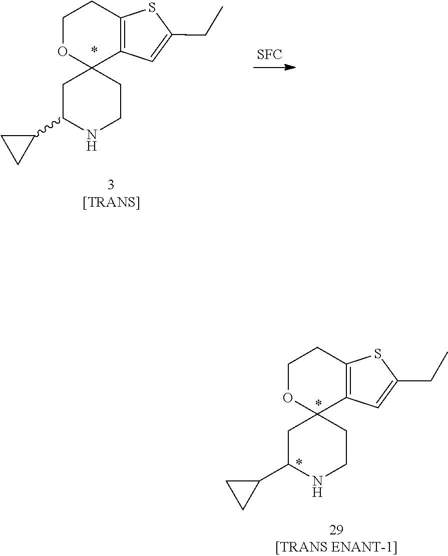

2′-cyclopropyl-2-ethyl-spiro[6,7-dihydrothieno[3,2-c]pyran-4,4′-piperidine]3 (24 mg, 0.07600 mmol) was separated into constituent enantiomers by chiral SFC separation. Column: Daicel Chiralpak @AD-H, 20×250 mm; Mobile Phase: 40% Methanol (5 mM ammonia), 60% carbon dioxide. Peak A was concentrated via rotovap to afford 2′-cyclopropyl-2-ethyl-spiro[6,7-dihydrothieno[3,2-c]pyran-4,4′-piperidine]29 (4.6 mg, 38%) as a single enantiomer. ¹H NMR (400 MHz, Chloroform-d) δ 6.54 (d, J=1.2 Hz, 1H), 3.98-3.75 (m, 2H), 3.19 (d, J=10.8 Hz, 2H), 2.88-2.64 (m, 4H), 2.46 (td, J=10.3, 3.8 Hz, 1H), 2.19-2.00 (m, 3H), 1.99-1.87 (m, 1H), 1.29 (t, J=7.5 Hz, 3H), 1.07-0.78 (m, 2H), 0.65-0.35 (m, 3H), 0.34-0.18 (m, 1H). LCMS m/z 278.17 [M+H]⁺.

### Preparation S32

### tert-butyl (2S,4S)-2′-bromo-2-(1-methyl-1H-1,2,3-triazol-4-yl)-4′,5′-dihydrospiro[piperidine-4,7′-thieno[2,3-c]pyran]-1-carboxylate (S32)

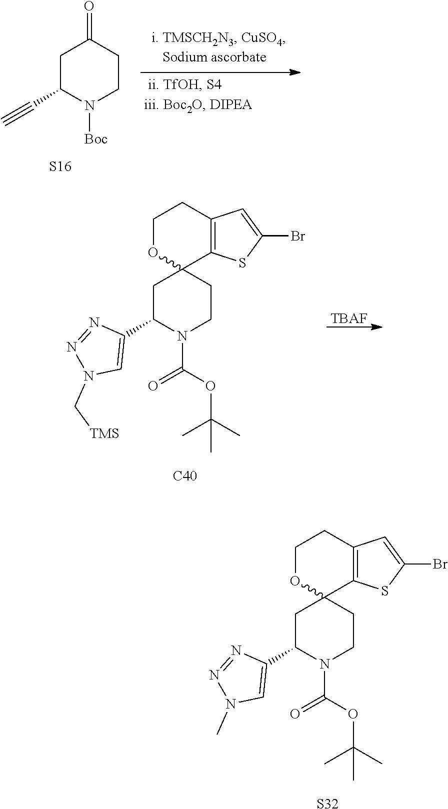

### Step 1. Synthesis of tert-butyl (2S)-2′-bromo-2-(1-((trimethylsilyl)methyl)-1H-1,2,3-triazol-4-yl)-4′,5′-dihydrospiro[piperidine-4,7′-thieno[2,3-c]pyran]-1-carboxylate (C40)

A mixture of tert-butyl (S)-2-ethynyl-4-oxopiperidine-1-carboxylate S16 (400 mg, 1.792 mmol), azidomethyl(trimethyl)silane (248 mg, 1.919 mmol), copper(II) sulfate (86 mg, 0.5388 mmol), and sodium ascorbate (316 mg, 1.794 mmol) in DMF (16 mL) was heated to 50° C. The solution was stirred overnight and then the mixture was cooled to room temperature diluted with ethyl acetate and water. The organic layer was washed with water (5×), brine, dried over sodium sulfate and concentrated in vacuo to give crude tert-butyl (S)-4-oxo-2-[1-(trimethylsilylmethyl)triazol-4-yl]piperidine-1-carboxylate (631 mg). LCMS m/z 353.27 [M+H]⁺. This material was dissolved in dioxane (10.8 mL) and 2-(5-bromo-3-thienyl)ethanol S4 (510 mg, 2.463 mmol) was added. The reaction was placed in an ice bath and triflic acid (952 μL, 10.74 mmol) was added. The reaction was allowed to warm to room temperature overnight. The reaction mixture was diluted with DCM and washed with sat. sodium bicarbonate and brine. The organic layer was dried over sodium sulfate and concentrated in vacuo. The crude product, (2S)-2′-bromo-2-(1-((trimethylsilyl)methyl)-1H-1,2,3-triazol-4-yl)-4′,5′-dihydrospiro[piperidine-4,7′-thieno[2,3-c]pyran](791 mg, 100%); LCMS m/z 441.12 [M+H]⁺ was redissolved in DCM (10.8 mL) and treated with Boc₂O (782 mg, 3.583 mmol) and DIPEA (937 μL, 5.377 mmol). After two hours the solvent was removed in vacuo. Purification by silica gel chromatography (Gradient: 0-30% EtOAc in heptane) yielded the product (2S)-2′-bromo-2-(1-((trimethylsilyl)methyl)-1H-1,2,3-triazol-4-yl)-4′,5′-dihydrospiro[piperidine-4,7′-thieno[2,3-c]pyran]-1-carboxylate C40 (660 mg, 68%). LCMS m/z 541.21 [M+H]⁺.

### Step 2. Synthesis of tert-butyl (2S)-2′-bromo-2-(1-methyl-1H-1,2,3-triazol-4-yl)-4′,5′-dihydrospiro[piperidine-4,7′-thieno[2,3-c]pyran]-1-carboxylate (S32)

To a solution of (2S)-2′-bromo-2-(1-((trimethylsilyl)methyl)-1H-1,2,3-triazol-4-yl)-4′,5′-dihydrospiro[piperidine-4,7′-thieno[2,3-c]pyran]-1-carboxylate C40 (660 mg, 1.219 mmol) in THF (9.9 mL) was added TBAF (1.4 mL of 1 M, 1.400 mmol) at 0° C. After 30 min the reaction was quenched with water, diluted with DCM, and the organic layer collected through a phase separator and concentrated in vacuo. Purification by silica gel chromatography (Gradient: 0-50% EtOAc in heptane) yielded the product tert-butyl (2S)-2′-bromo-2-(1-methyl-1H-1,2,3-triazol-4-yl)-4′,5′-dihydrospiro[piperidine-4,7′-thieno[2,3-c]pyran]-1-carboxylate S32 (390 mg, 57%) as a 3:1 mixture of diastereomers. LCMS m/z 469.16 [M+H]⁺.

### Compounds 30 and 31

### (2S)-2′-cyclopropyl-2-(1-methyl-1H-1,2,3-triazol-4-yl)-4′,5′-dihydrospiro[piperidine-4,7′-thieno[2,3-c]pyran](30) and (2S,4S)-2′-cyclopropyl-2-(1-methyl-]H-1,2,3-triazol-4-yl)-4′,5′-dihydrospiro[piperidine-4,7′-thieno[2,3-c]pyran] (31)

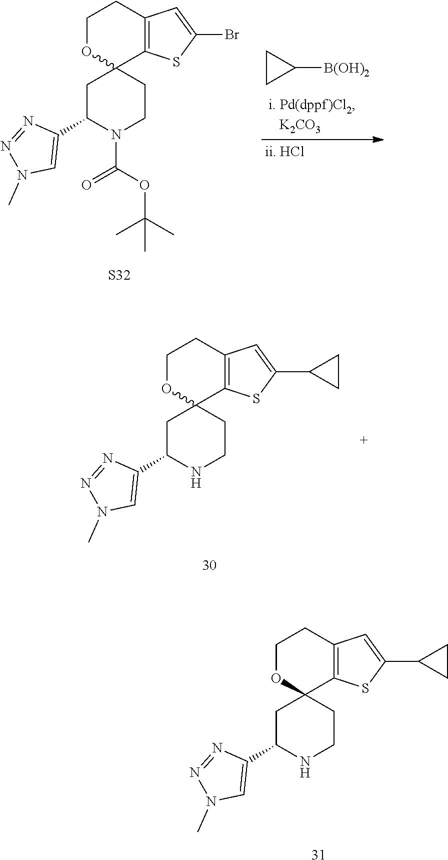

### Preparation of (2S)-2′-cyclopropyl-2-(1-methyl-1H-1,2,3-triazol-4-yl)-4′,5′-dihydrospiro[piperidine-4,7′-thieno[2,3-c]pyran](30) and (2S,4S)-2′-cyclopropyl-2-(1-methyl-1H-1,2,3-triazol-4-yl)-4′,5′-dihydrospiro[piperidine-4,7′-thieno[2,3-c]pyran] (31)

Tert-butyl (2S)-2′-bromo-2-(1-methyl-1H-1,2,3-triazol-4-yl)-4′,5′-dihydrospiro[piperidine-4,7′-thieno[2,3-c]pyran]-1-carboxylate S32 (117 mg, 0.2069 mmol), cyclopropylboronic acid (22 mg, 0.2561 mmol), Pd(dppf)Cl₂ (17 mg, 0.02082 mmol) and K₂CO₃ (57 mg, 0.4124 mmol) was brought up in DME (1.7 mL) and the mixture was sparged with nitrogen for 5 minutes before heating to reflux overnight. The reaction was diluted with DCM and water and the organic layer was separated through a phase separator and dried via in vacuo. Purification by reversed-phase chromatography (Gradient: 0-100% MeCN in water with 0.1% TFA) afforded tert-butyl (2S)-2′-cyclopropyl-2-(1-methyl-1H-1,2,3-triazol-4-yl)-4′,5′-dihydrospiro[piperidine-4,7′-thieno[2,3-c]pyran]-1-carboxylate (35 mg, 30%). LCMS m/z 431.35 [M+H]⁺. This intermediate was then dissolved in HCl (720 μL of 4 M in dioxane, 2.880 mmol). After stirring for 30 minutes the reaction mixture was concentrated via rotovap. Purification by reversed-phase HPLC (Method: C18 Waters Sunfire column (30×150 mm, 5 micron). Gradient: MeCN in H₂O with 0.1% trifluoroacetic acid) afforded one tube of a 60:40 mixture of diastereomers (2S)-2′-cyclopropyl-2-(1-methyl-1H-1,2,3-triazol-4-yl)-4′,5′-dihydrospiro[piperidine-4,7′-thieno[2,3-c]pyran]30 (10.50 mg, 11%) as the trifluoroacetate salt. ¹H NMR (400 MHz, Chloroform-d) δ 7.90 (d, J=5.3 Hz, 1H), 6.49 (dd, J=14.1, 0.8 Hz, 1H), 5.12-4.84 (m, 1H), 4.12 (d, J=8.9 Hz, 3H), 4.03-3.73 (m, 2H), 3.54 (dd, J=45.9, 12.9 Hz, 2H), 3.01-2.15 (m, 6H), 2.11-1.95 (m, 1H), 1.14-0.91 (m, 2H), 0.80-0.59 (m, 2H). Another tube of a single diastereomer, the major product, afforded (2S,4S)-2′-cyclopropyl-2-(1-methyl-1H-1,2,3-triazol-4-yl)-4′,5′-dihydrospiro[piperidine-4,7′-thieno[2,3-c]pyran]31 (5 mg, 10%) as a trifluoroacetate salt. ¹H NMR (400 MHz, Chloroform-d) δ 9.69 (d, J=116.3 Hz, 2H), 7.89 (s, 1H), 6.47 (d, J=0.8 Hz, 1H), 4.93 (d, J=12.3 Hz, 1H), 4.10 (s, 3H), 3.98-3.91 (m, 2H), 3.66-3.55 (m, 1H), 3.48 (d, J=12.4 Hz, 1H), 2.68-2.62 (m, 3H), 2.43-2.28 (m, 2H), 2.21 (d, J=14.7 Hz, 1H), 2.03 (ttd, J=8.3, 5.0, 0.8 Hz, 1H), 1.06-0.95 (m, 2H), 0.74-0.65 (m, 2H).

### Compound 32

### (2S)-2′-methyl-2-(1-methyl-1H-1,2,3-triazol-4-yl)-4′,5′-dihydrospiro[piperidine-4,7′-thieno[2,3-c]pyran] (32)

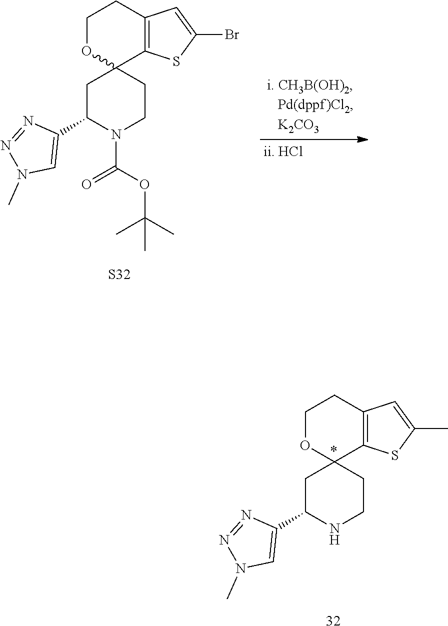 [SMILES: Cn1cc([C@@H]2CC3(CCN2C(=O)OC(C)(C)C)OCCc2cc(Br)sc23)nn1 ; "S32" ; Cc1cc2c(s1)C1(CCN[C@H](c3cn(C)nn3)C1)OCC2 ; "32"]

### Preparation of (2S,4S)-2′-methyl-2-(1-methyl-1H-1,2,3-triazol-4-yl)-4′,5′-dihydrospiro[piperidine-4,7′-thieno[2,3-c]pyran](32)

This compound was made following the conditions for Compound 31 but using methylboronic acid. (2S)-2′-methyl-2-(1-methyl-1H-1,2,3-triazol-4-yl)-4′,5′-dihydrospiro[piperidine-4,7′-thieno[2,3-c]pyran]32 (3.5 mg, 10%), a single enantiomer, was afforded as the trifluoroacetate salt. ¹H NMR (400 MHz, Chloroform-d) δ 9.74 (d, J=118.8 Hz, 2H), 7.88 (s, 1H), 6.47 (d, J=1.2 Hz, 1H), 4.93 (d, J=12.3 Hz, 1H), 4.10 (s, 3H), 3.96 (t, J=5.5 Hz, 2H), 3.61 (t, J=12.6 Hz, 1H), 3.48 (d, J=12.2 Hz, 1H), 2.70-2.58 (m, 3H), 2.49-2.37 (m, 4H), 2.36-2.28 (m, 1H), 2.22 (d, J=14.6 Hz, 1H). LCMS m/z 305.16 [M+H]⁺.

### Compound 33

### (2S,4S)-2′-iodo-2-(1-methyl-1H-1,2,3-triazol-4-yl)-4′,5′-dihydrospiro[piperidine-4,7′-thieno[2,3-c]pyran] (33)

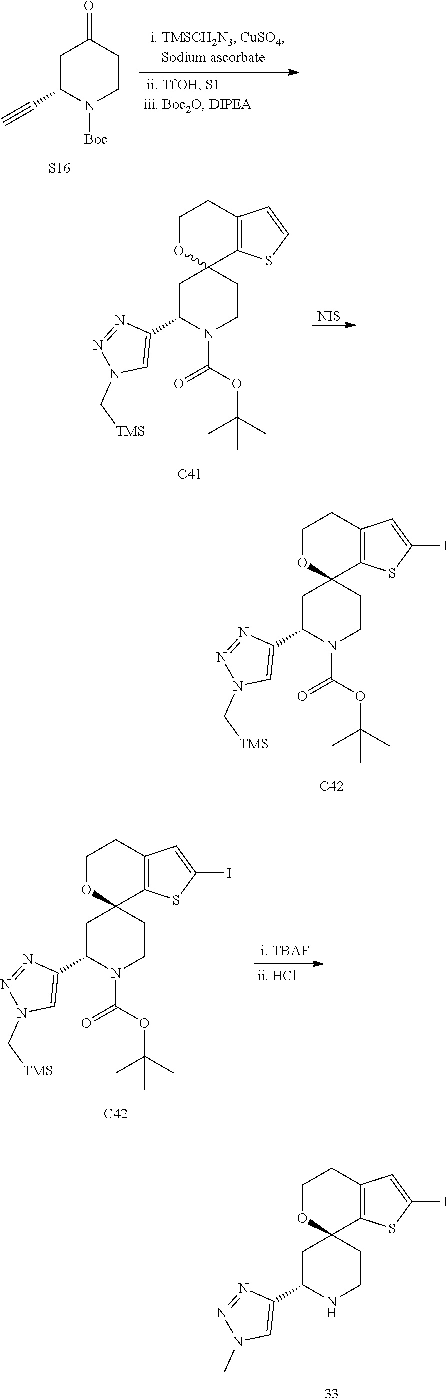

### Step 1. Synthesis of tert-butyl (2S)-2-(1-((trimethylsilyl)methyl)-1H-1,2,3-triazol-4-yl)-4′,5′-dihydrospiro[piperidine-4,7′-thieno[2,3-c]pyran]-1-carboxylate (C41)

A mixture of tert-butyl (S)-2-ethynyl-4-oxopiperidine-1-carboxylate S16 (500 mg, 2.239 mmol), azidomethyl(trimethyl)silane (356 μL, 2.397 mmol), copper(II) sulfate (107 mg, 0.6704 mmol), sodium ascorbate (400 mg, 2.271 mmol) in DMF (20 mL) was heated to 50° C. After stirring overnight, the mixture was cooled to room temperature, concentrated, and redissolved in DCM/water. The organic layer was collected through a phase separator and concentrated to give crude (S)-4-oxo-2-[1-(trimethylsilylmethyl)triazol-4-yl]piperidine-1-carboxylate (789 mg, 100%). LCMS 353.23 [M+H]⁺. This intermediate was brought up in dioxane (10 mL). 2-(3-thienyl)ethanol S1 (574 mg, 4.478 mmol) was added and the reaction was cooled to 0° C. Triflic acid (792 μL, 8.94 mmol) was added and the reaction was warmed to room temperature. After one hour the mixture was diluted with DCM and washed with sat. sodium bicarbonate and brine. The organic layer was dried over sodium sulfate and concentrated in vacuo to give crude (2S)-2-(1-((trimethylsilyl)methyl)-1H-1,2,3-triazol-4-yl)-4′,5′-dihydrospiro[piperidine-4,7′-thieno[2,3-c]pyran], LCMS m/z 363.22 [M+H]⁺, which was immediately re-dissolved in DCM (10 mL) and treated with DIPEA (1.17 mL, 6.716 mmol) and Boc₂O (977 mg, 4.477 mmol). After 30 minutes the solvent was removed in vacuo. Purification by silica gel chromatography (Gradient: 0-30% EtOAc in heptane) yielded tert-butyl (2S)-2-(1-((trimethylsilyl)methyl)-1H-1,2,3-triazol-4-yl)-4′,5′-dihydrospiro[piperidine-4,7′-thieno[2,3-c]pyran]-1-carboxylate C41 (510 mg, 49%) as a mixture of diastereomers. LCMS m/z 463.31 [M+H]⁺.

### Step 2. Synthesis of tert-butyl (2S,4S)-2′-iodo-2-(1-((trimethylsilyl)methyl)-1H-1,2,3-triazol-4-yl)-4′,5′-dihydrospiro[piperidine-4,7′-thieno[2,3-c]pyran]-1-carboxylate (C42)

A solution of tert-butyl (2S)-2-(1-((trimethylsilyl)methyl)-1H-1,2,3-triazol-4-yl)-4′,5′-dihydrospiro[piperidine-4,7′-thieno[2,3-c]pyran]-1-carboxylate C41 (475 mg, 1.027 mmol) and NIS (305 mg, 1.356 mmol) in chloroform (3.4 mL) and acetic acid (1.1 mL) was stirred at room temperature overnight. The reaction was quenched with sat. sodium bicarbonate solution and diluted with DCM. The organic layer was collected via filtration through a phase separator and the solvent was removed in vacuo. Purification by silica gel chromatography (Gradient: 0-50% EtOAc in heptane) yielded the desired diastereomer as Peak B, tert-butyl (2S,4S)-2′-iodo-2-(1-((trimethylsilyl)methyl)-1H-1,2,3-triazol-4-yl)-4′,5′-dihydrospiro[piperidine-4,7′-thieno[2,3-c]pyran]-1-carboxylate C42 (370 mg, 61%). ¹H NMR (400 MHz, Chloroform-d) δ 7.30 (s, 1H), 6.88 (s, 1H), 5.25 (dd, J=9.9, 6.5 Hz, 1H), 3.96-3.89 (m, 1H), 3.89-3.85 (m, 4H), 3.43 (ddd, J=13.9, 9.6, 5.7 Hz, 1H), 2.65-2.54 (m, 3H), 2.46 (ddd, J=14.5, 6.5, 1.7 Hz, 1H), 2.26 (dt, J=15.5, 7.8 Hz, 1H), 1.92 (ddd, J=14.5, 5.7, 3.9 Hz, 1H), 1.42 (s, 9H), 0.14 (s, 9H).

### Step 3. Synthesis of (2S,4S)-2′-iodo-2-(J-methyl-1H-1,2,3-triazol-4-yl)-4′,5′-dihydrospiro[piperidine-4,7′-thieno[2,3-c]pyran](33)

To a solution of tert-butyl (2S,4S)-2′-iodo-2-(1-((trimethylsilyl)methyl)-1H-1,2,3-triazol-4-yl)-4′,5′-dihydrospiro[piperidine-4,7′-thieno[2,3-c]pyran]-1-carboxylate C42 (240 mg, 0.4078 mmol) in THF (3.6 mL) was added TBAF (110 μL, 0.3732 mmol). The reaction was stirred at room temperature for 5 hours and then quenched with sat. sodium bicarbonate solution and DCM. The organics were separated, dried over sodium sulfate, and concentrated in vacuo. Purification by silica gel chromatography (Gradient: 0-50% EtOAc in heptane) yielded the product tert-butyl (2S,4S)-2′-iodo-2-(1-methyl-1H-1,2,3-triazol-4-yl)-4′,5′-dihydrospiro[piperidine-4,7′-thieno[2,3-c]pyran]-1-carboxylate (160 mg, 57%). LCMS m/z 517.26 [M+H]⁺. 50 mg of this intermediate was dissolved in dioxane (1.2 mL) and treated with HCl (1 mL of 4 M in dioxane, 4.000 mmol). After 30 minutes the solvent was removed in vacuo. Purification by reversed-phase HPLC. Method: C18 Waters Sunfire column (30×150 mm, 5 micron). Gradient: MeCN in H₂O with 0.1% trifluoroacetic acid. (2S,4S)-2′-iodo-2-(1-methyl-1H-1,2,3-triazol-4-yl)-4′,5′-dihydrospiro[piperidine-4,7′-thieno[2,3-c]pyran]33 was afforded as the trifluoroacetate salt (12 mg, 55%). ¹H NMR (400 MHz, Chloroform-d) δ 9.69 (d, J=115.5 Hz, 2H), 7.87 (s, 1H), 6.96 (s, 1H), 4.93 (d, J=12.3 Hz, 1H), 4.09 (s, 3H), 3.92 (td, J=5.6, 2.6 Hz, 2H), 3.68-3.37 (m, 2H), 2.82-2.50 (m, 3H), 2.50-2.03 (m, 3H). LCMS m/z 416.85 [M+H]⁺.

### Compound 34

### (2S,4S)-2′-chloro-2-(3-methylisoxazol-5-yl)-4′,5′-dihydrospiro[piperidine-4,7′-thieno[2,3-c]pyran]-4′-ol (34)

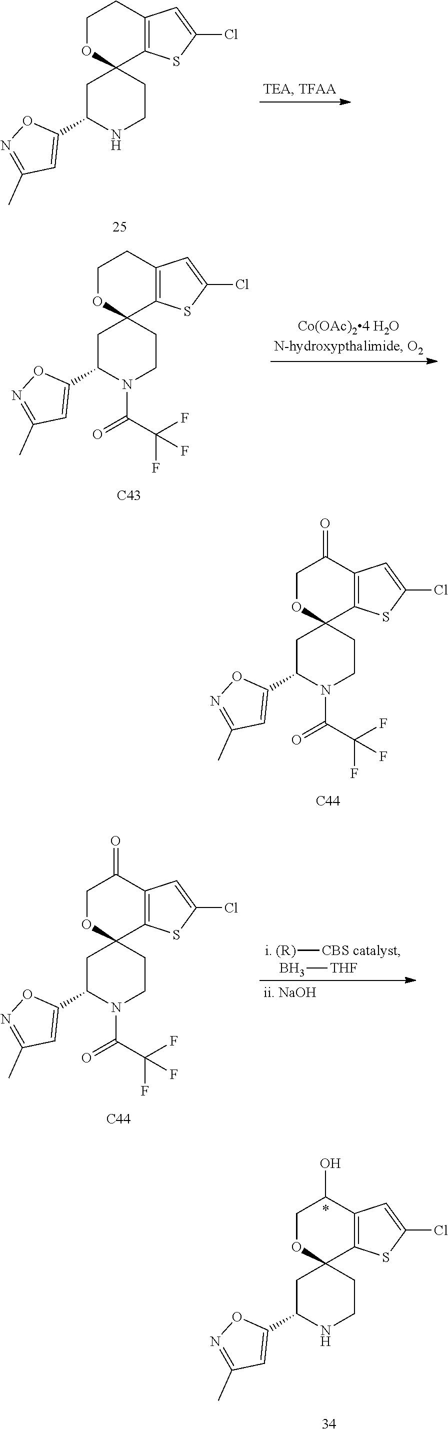

### Step 1. Synthesis of 1-((2S,4S)-2′-chloro-2-(3-methylisoxazol-5-yl)-4′,5′-dihydrospiro[piperidine-4,7′-thieno[2,3-c]pyran]-1-yl)-2,2,2-trifluoroethan-]-one (C43)

To a solution of (2S,4S)-2′-chloro-2-(3-methylisoxazol-5-yl)-4′,5′-dihydrospiro[piperidine-4,7′-thieno[2,3-c]pyran]25 (150 mg, 0.4525 mmol) in DCM (2 mL) was added TEA (130 μL, 0.9327 mmol) and TFAA (90 μL, 0.6475 mmol). The reaction mixture was stirred for 2 hours, diluted with EtOAc and saturated NaHCO₃ and the organic layer dried and concentrated to an oil. Purification by silica gel chromatography (Gradient: 0-60% EtOAc in heptane) afforded the product 1-((2S,4S)-2′-chloro-2-(3-methylisoxazol-5-yl)-4′,5′-dihydrospiro[piperidine-4,7′-thieno[2,3-c]pyran]-1-yl)-2,2,2-trifluoro-ethanone C43 (160 mg, 84%) as a white foam. LCMS m/z 421.09 [M+H]⁺.

### Step 2. Synthesis of(2S,4S)-2′-chloro-2-(3-methylisoxazol-5-yl)-1-(2,2,2-trifluoroacetyl)spiro[piperidine-4,7′-thieno[2,3-c]pyran]-4′(5′H)-one (C44)

A solution of 1-((2S,4S)-2′-chloro-2-(3-methylisoxazol-5-yl)-4′,5′-dihydrospiro[piperidine-4,7′-thieno[2,3-c]pyran]-1-yl)-2,2,2-trifluoro-ethanone C43 (160 mg, 0.3802 mmol), cobalt(II) acetate tetrahydrate (10 mg, 0.04015 mmol), and N-hydroxyphthalimide (25 mg, 0.1533 mmol) in ACN (3 mL) was vacuum purged with an oxygen balloon three times. The flask was heated to 60° C. under an oxygen atmosphere. After 4 hours the reaction mixture was diluted with water and EtOAc and the organic layer dried and concentrated to an oil. Purification by silica gel chromatography (Gradient: 0-80% EtOAc in heptane) afforded (2S,4S)-2′-chloro-2-(3-methylisoxazol-5-yl)-1-(2,2,2-trifluoroacetyl)spiro[piperidine-4,7′-thieno[2,3-c]pyran]-4′(5′H)-one C44 (30 mg, 18%) as a white solid. ¹H NMR (400 MHz, Chloroform-d) δ 7.23 (s, 1H), 6.13 (s, 1H), 5.67-5.53 (m, 1H), 4.44-4.26 (m, 2H), 4.18-4.01 (m, 1H), 3.95-3.73 (m, 1H), 2.85-2.70 (m, 1H), 2.70-2.56 (m, 2H), 2.33 (s, 3H), 2.14-1.99 (m, 1H). LCMS m/z 435.04 [M+H]⁺.

### Step 3. Synthesis of (2S,4S)-2′-chloro-2-(3-methylisoxazol-5-yl)-4′,5′-dihydrospiro[piperidine-4,7′-thieno[2,3-c]pyran] (34)

To a solution of (R)-(+)-2-Methyl-CBS-oxazaborolidine solution (15 μL of 1 M in toluene, 0.0150 mmol) in MTBE (0.4 mL) at 0° C. was added borane tetrahydrofuran (140 μL of 1 M in THF, 0.1400 mmol). After 2 minutes, (2S,4S)-2′-chloro-2-(3-methylisoxazol-5-yl)-1-(2,2,2-trifluoroacetyl)spiro[piperidine-4,7′-thieno[2,3-c]pyran]-4′(5′H)-one C44 (30 mg, 0.06899 mmol) in MTBE (1 mL) was added and the reaction mixture was stirred at 0° C. for 1 hour. The reaction was quenched with 1 M HCl (0.3 mL) and stirred for 30 minutes. 6 M NaOH (0.5 mL) was added and the mixture was stirred overnight. The reaction mixture was diluted with EtOAc and water and the organic layer concentrated to an oil. Purification by reversed-phase HPLC (Method: C18 Waters Sunfire column (30×150 mm, 5 micron). Gradient: MeCN in H₂O with 10 mM ammonium hydroxide) afforded (2S,4S)-2′-chloro-2-(3-methylisoxazol-5-yl)-4′,5′-dihydrospiro[piperidine-4,7′-thieno[2,3-c]pyran]34 (20 mg, 82%) as an oil. ¹H NMR (400 MHz, Chloroform-d) δ 6.86 (s, 1H), 6.01 (s, 1H), 4.48 (t, J=2.9 Hz, 1H), 4.41 (dd, J=11.6, 2.6 Hz, 1H), 3.99 (qd, J=12.4, 2.8 Hz, 2H), 3.20 (td, J=12.4, 2.6 Hz, 1H), 3.06 (ddd, J=12.2, 4.8, 2.2 Hz, 1H), 2.30 (s, 3H), 2.20-2.09 (m, 1H), 1.98-1.85 (m, 2H), 1.76-1.64 (m, 2H). LCMS m/z 341.03 [M+H]⁺.

### Preparation S33

### (2S,4S)-2′-chloro-2-(1-methyl-1H-1,2,3-triazol-4-yl)-1-(2,2,2-trifluoroacetyl)spiro[piperidine-4,7′-thieno[2,3-c]pyran]-4′(5′H)-one (S33)

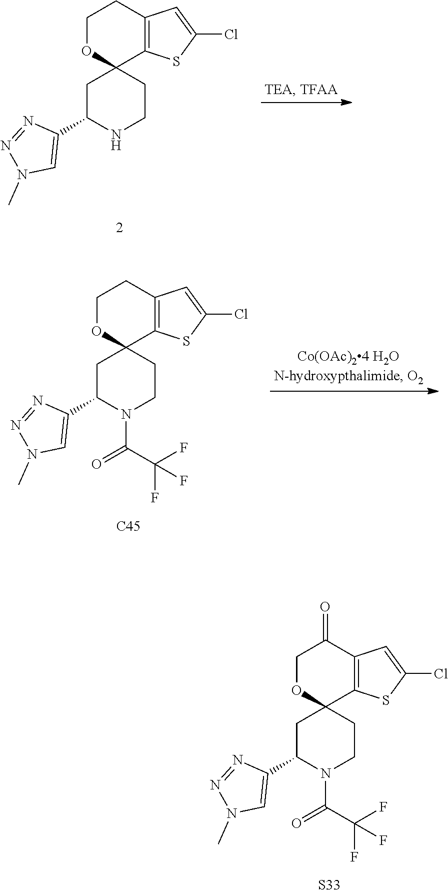 [SMILES: Cn1cc([C@@H]2C[C@]3(CCN2)OCCc2cc(Cl)sc23)nn1 ; Cn1cc([C@@H]2C[C@]3(CCN2C(=O)C(F)(F)F)OCCc2cc(Cl)sc23)nn1 ; "C45" ; Cn1cc([C@@H]2C[C@]3(CCN2C(=O)C(F)(F)F)OCC(=O)c2cc(Cl)sc23)nn1 ; "S33"]

### Step 1. Synthesis of 1-((2S,4S)-2′-chloro-2-(1-methyl-1H-1,2,3-triazol-4-yl)-4′,5′-dihydrospiro[piperidine-4,7′-thieno[2,3-c]pyran]-1-yl)-2,2,2-trifluoroethan-]-one (C45)

A solution of (2S,4S)-2′-chloro-2-(1-methyl-1H-1,2,3-triazol-4-yl)-4′,5′-dihydrospiro[piperidine-4,7′-thieno[2,3-c]pyran]2 (2.2 g, 6.613 mmol) in DCM (30 mL) was treated with TEA (3.6 mL, 25.83 mmol) followed by TFAA (1.1 mL, 7.914 mmol). After 20 minutes the reaction mixture was diluted with sat. sodium bicarbonate and DCM. The organics were separated via a phase separator and concentrated in vacuo to afford 1-((2S,4S)-2′-chloro-2-(1-methyl-1H-1,2,3-triazol-4-yl)-4′,5′-dihydrospiro[piperidine-4,7′-thieno[2,3-c]pyran]-1-yl)-2,2,2-trifluoroethan-1-one C45 (2.8 g, 84%) as a yellow semi-solid. ¹H NMR (400 MHz, Chloroform-d) δ 7.60 (s, 1H), 6.58 (s, 1H), 5.51 (t, J=9.2 Hz, 1H), 4.08 (s, 3H), 3.97-3.80 (m, 4H), 2.94 (t, J=13.1 Hz, 1H), 2.59 (t, J=5.5 Hz, 2H), 2.44 (ddd, J=34.2, 17.1, 8.0 Hz, 2H), 2.07 (d, J=14.7 Hz, 1H). ¹⁹F NMR (376 MHz, Chloroform-d) 6-69.99. LCMS m/z 421.14 [M+H]⁺.

### Step 2. Synthesis of(2S,4S)-2′-chloro-2-(1-methyltriazol-4-yl)-1-(2,2,2-trifluoroacetyl)spiro[piperidine-4,7′-thieno[2,3-c]pyran]-4′-one (S33)

To a solution of 1-((2S,4S)-2′-chloro-2-(1-methyl-1H-1,2,3-triazol-4-yl)-4′,5′-dihydrospiro[piperidine-4,7′-thieno[2,3-c]pyran]-1-yl)-2,2,2-trifluoroethan-1-one C45 (660 mg, 1.556 mmol) in ACN (18 mL) was added cobalt(II) acetate tetrahydrate (70 mg, 0.2810 mmol) and N-hydroxyphthalimide (135 mg, 0.8276 mmol). The reaction was purged and evacuated with oxygen (3×) and heated to 45° C. under an oxygen balloon. After 2.5 hrs the reaction was diluted with water and partitioned with DCM. The organics were collected via filtration through a phase separator and then concentrated via rotovap. Purification by silica gel chromatography (Gradient: 0-65% EtOAc in Heptane) afforded (2S,4S)-2′-chloro-2-(1-methyltriazol-4-yl)-1-(2,2,2-trifluoroacetyl)spiro[piperidine-4,7′-thieno[2,3-c]pyran]-4′-one S33 (366 mg, 49%) as a white foam. ¹H NMR (300 MHz, Chloroform-d) δ 7.63 (s, 1H), 7.19 (s, 1H), 5.55 (t, J=8.8 Hz, 1H), 4.44-4.24 (m, 2H), 4.09 (s, 3H), 3.91 (d, J=15.7 Hz, 2H), 3.13 (dd, J=14.7, 10.7 Hz, 1H), 2.75-2.53 (m, 2H), 2.18-2.02 (m, 1H). LCMS m/z 435.04 [M+H]⁺.

### Compound 35

### (2S,4S)-2′-chloro-2-(1-methyl-1H-1,2,3-triazol-4-yl)-4′,5′-dihydrospiro[piperidine-4,7′-thieno[2,3-c]pyran]-4′-ol (35)

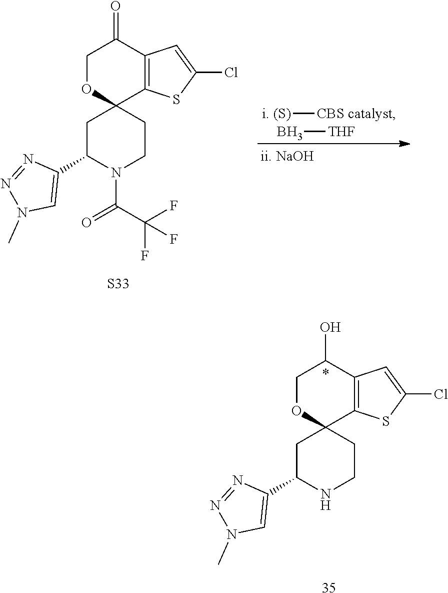

### Synthesis of (2S,4S)-2′-chloro-2-(1-methyl-1H-1,2,3-triazol-4-yl)-4′,5′-dihydrospiro[piperidine-4,7′-thieno[2,3-c]pyran]-4′-ol (35)

A solution of (S)-(−)-2-Methyl-CBS-oxazaborolidine solution (145 μL of 1 M in THF, 0.1450 mmol) in MTBE (4 mL) was cooled to 0° C. and treated with borane tetrahydrofuran (1000 μL of 1 M in THF, 1.00 mmol). Then a solution of (2S,4S)-2′-chloro-2-(1-methyltriazol-4-yl)-1-(2,2,2-trifluoroacetyl)spiro[piperidine-4,7′-thieno[2,3-c]pyran]-4′-one S33 (200 mg, 0.4172 mmol) in MTBE (1.5 mL) was added and the reaction was stirred at 0° C. After 15 minutes the reaction was quenched with 2 M HCl, the ice bath was removed, and the partitioned mixture stirred vigorously overnight. The reaction was diluted with DCM and organics collected via a phase separator and concentrated via rotovap. Purification by silica gel chromatography (Gradient: 0-70% EtOAc in Heptane) afforded 1-((2S,4S)-2′-chloro-4′-hydroxy-2-(1-methyl-1H-1,2,3-triazol-4-yl)-4′,5′-dihydrospiro[piperidine-4,7′-thieno[2,3-c]pyran]-1-yl)-2,2,2-trifluoroethan-1-one (140 mg, 74%) as a white foam. ¹H NMR (300 MHz, Chloroform-d) δ 7.62 (s, 1H), 6.86 (s, 1H), 5.53-5.41 (m, 1H), 4.47 (dt, J=9.4, 3.3 Hz, 1H), 4.10 (s, 3H), 3.94 (qd, J=12.3, 3.3 Hz, 4H), 2.91 (t, J=13.1 Hz, 1H), 2.65-2.38 (m, 2H), 2.19 (d, J=14.4 Hz, 1H), 2.07 (d, J=3.8 Hz, 1H). ¹⁹F NMR (282 MHz, Chloroform-d) δ −69.96. LCMS m/z 437.11 [M+H]⁺. This material was then brought up in MeOH (3 mL) and treated with NaOH (2 mL of 2 M, 4.000 mmol) at room temperature. After 5 min the reaction was diluted with water and DCM and filtered through a phase separator to elute the organics, which were concentrated in vacuo. This residue was brought up in DCM (3 mL) and HCl (95 μL of 4 M, 0.3800 mmol) was added dropwise. The material was concentrated, redissolved in MeOH, and concentrated again (2×). The pale yellow film was then dissolved in water, transferred to a vial, frozen in a −78° C. dry ice bath and lyophilized over the weekend. (2S,4S)-2′-chloro-2-(1-methyl-1H-1,2,3-triazol-4-yl)-4′,5′-dihydrospiro[piperidine-4,7′-thieno[2,3-c]pyran]-4′-ol 35 (97.1 mg, 59%), a hydrochloride salt, was afforded as a yellow solid. ¹H NMR (300 MHz, Methanol-d₄) δ 8.07 (s, 1H), 6.94 (s, 1H), 4.83 (dd, J=12.5, 3.2 Hz, 1H), 4.51 (t, J=3.8 Hz, 1H), 4.14-4.04 (m, 4H), 3.87 (dd, J=12.2, 4.1 Hz, 1H), 3.59 (td, J=13.1, 3.3 Hz, 1H), 3.49-3.38 (m, 1H), 2.62 (dt, J=14.8, 2.9 Hz, 1H), 2.40 (dd, J=14.7, 2.8 Hz, 1H), 2.31 (dd, J=14.9, 12.6 Hz, 1H), 2.15 (td, J=14.0, 4.8 Hz, 1H). LCMS m/z 340.98 [M+H]⁺.

### Compound 36

### (2S,4S)-2′-chloro-2-(1-methyl-1H-1,2,3-triazol-4-yl)-4′,5′-dihydrospiro[piperidine-4,7′-thieno[2,3-c]pyran]-4′-ol (36)

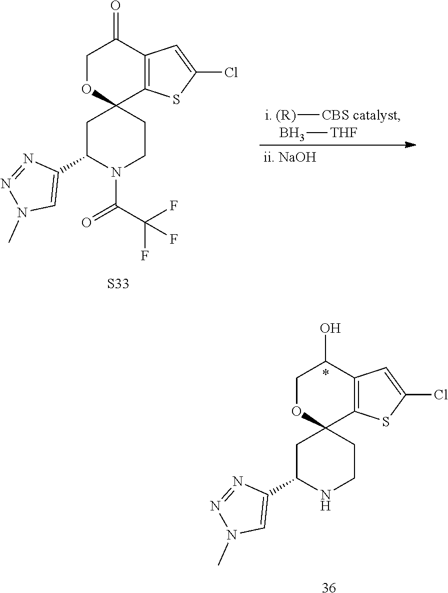

### Synthesis of (2S,4S)-2′-chloro-2-(1-methyl-1H-1,2,3-triazol-4-yl)-4′,5′-dihydrospiro[piperidine-4,7′-thieno[2,3-c]pyran]-4′-ol (36)

This compound was made following the conditions for Compound 35 but using (R)-(+)-2-Methyl-CBS-oxazaborolidine solution (1 M in toluene). (2S,4S)-2′-chloro-2-(1-methyl-1H-1,2,3-triazol-4-yl)-4′,5′-dihydrospiro[piperidine-4,7′-thieno[2,3-c]pyran]-4′-ol hydrochloride 36 (134 mg, 78%) was afforded as a white solid. ¹H NMR (300 MHz, Methanol-d₄) δ 8.07 (s, 1H), 6.95 (s, 1H), 4.91 (dd, J=12.4, 3.2 Hz, 1H), 4.51 (t, J=3.5 Hz, 1H), 4.13 (s, 3H), 3.98 (ddd, J=51.0, 12.3, 3.5 Hz, 2H), 3.60-3.37 (m, 2H), 2.61 (dt, J=14.5, 2.9 Hz, 1H), 2.47-2.34 (m, 2H), 2.04 (ddd, J=15.0, 13.2, 4.9 Hz, 1H). LCMS m/z 341.07 [M+H]⁺.

### Compounds 37 and 38

### (2S,4S)-2′-chloro-4′-methyl-2-(1-methyl-1H-1,2,3-triazol-4-yl)-4′,5′-dihydrospiro[piperidine-4,7′-thieno[2,3-c]pyran]-4′-ol (37)[DIAST-1] and (38)[DIAST-2]

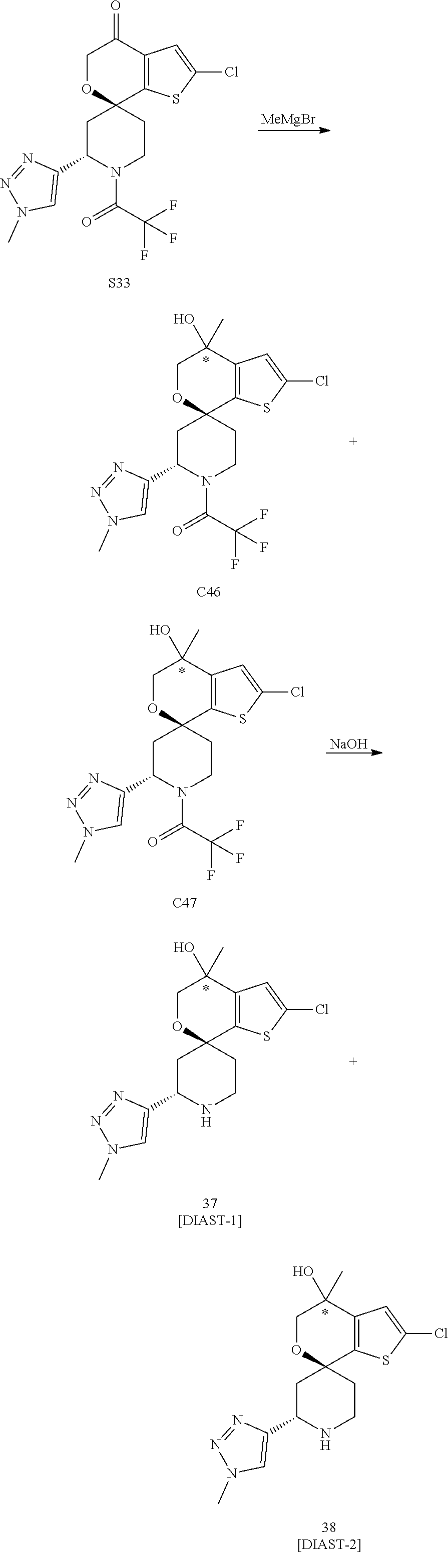

### Step 1. Synthesis of 1-((2S,4S)-2′-chloro-4′-hydroxy-4′-methyl-2-(1-methyl-1H-1,2,3-triazol-4-yl)-4′,5′-dihydrospiro[piperidine-4,7′-thieno[2,3-c]pyran]-1-yl)-2,2,2-trifluoroethan-]-one (C46)[DIAST-1] and (C47)[DIAST-2]

(2S,4S)-2′-chloro-2-(1-methyltriazol-4-yl)-1-(2,2,2-trifluoroacetyl)spiro[piperidine-4,7′-thieno[2,3-c]pyran]-4′-one S33 (30 mg, 0.06565 mmol) in 2-MeTHF (1 mL) was cooled to 0° C. and treated with MeMgBr (26 μL of 3.4 M in 2-MeTHF, 0.08840 mmol) dropwise. After 15 min at 0° C. the reaction was quenched with sat. ammonium chloride, diluted with water, and extracted with DCM through a phase separator. The organics were concentrated via rotovap. Purification by silica gel chromatography (Gradient: 0-80% EtOAc in Heptane) afforded 1-((2S,4S)-2′-chloro-4′-hydroxy-4′-methyl-2-(1-methyl-1H-1,2,3-triazol-4-yl)-4′,5′-dihydrospiro[piperidine-4,7′-thieno[2,3-c]pyran]-1-yl)-2,2,2-trifluoroethan-1-one C46[DIAST-1] (8 mg, 26%) as a white solid. LCMS m/z 451.24 [M+H]⁺. The second-eluting peak afforded 1-((2S,4S)-2′-chloro-4′-hydroxy-4′-methyl-2-(1-methyl-1H-1,2,3-triazol-4-yl)-4′,5′-dihydrospiro[piperidine-4,7′-thieno[2,3-c]pyran]-1-yl)-2,2,2-trifluoroethan-1-one C47[DIAST-2] (9 mg, 29%) as a clear film. ¹H NMR (300 MHz, Chloroform-d) δ 7.61 (s, 1H), 6.86 (s, 1H), 5.59 (t, J=9.0 Hz, 1H), 4.08 (s, 3H), 3.89 (s, 2H), 3.82-3.67 (m, 2H), 3.03 (t, J=12.8 Hz, 1H), 2.48 (dt, J=18.5, 10.1 Hz, 2H), 2.27 (s, 1H), 1.96 (d, J=14.9 Hz, 1H), 1.44 (s, 3H). LCMS m/z 433.2 (M+H−18)⁺.

### Step 2. Synthesis of (2S,4S)-2′-chloro-4′-methyl-2-(1-methyl-1H-1,2,3-triazol-4-yl)-4′,5′-dihydrospiro[piperidine-4,7′-thieno[2,3-c]pyran]-4′-ol (37)[DIAST-1] and (38)[DIAST-2]

1-((2S,4S)-2′-chloro-4′-hydroxy-4′-methyl-2-(1-methyl-1H-1,2,3-triazol-4-yl)-4′,5′-dihydrospiro[piperidine-4,7′-thieno[2,3-c]pyran]-1-yl)-2,2,2-trifluoroethan-1-one (C46)[DIAST-1] (8 mg) and (C47)[DIAST-2] (9 mg) were brought up separately in MeOH (1 mL) and treated with NaOH (0.4 mL of 2 M, 0.8000 mmol). After 10 min at room temperature the reaction was diluted with water and extracted with DCM (2×) through a phase separator. The organics were concentrated in vacuo to afford each diastereomer. (2S,4S)-2′-chloro-4′-methyl-2-(1-methyl-1H-1,2,3-triazol-4-yl)-4′,5′-dihydrospiro[piperidine-4,7′-thieno[2,3-c]pyran]-4′-ol (37) [DIAST-1] (6.6 mg, 28%) was afforded as a white foam. 1H NMR (300 MHz, Chloroform-d) δ 7.44 (s, 1H), 6.86 (s, 1H), 4.33 (dd, J=11.7, 2.6 Hz, 1H), 4.07 (s, 3H), 3.80 (q, J=11.9 Hz, 2H), 3.31 (td, J=12.3, 2.9 Hz, 1H), 3.09-3.00 (m, 1H), 2.52-2.42 (m, 1H), 2.00 (m, J=2.6 Hz, 1H), 1.87 (td, J=13.0, 4.6 Hz, 1H), 1.72 (dd, J=13.9, 11.7 Hz, 1H), 1.43 (s, 3H). LCMS m/z 355.07 [M+H]⁺. (2S,4S)-2′-chloro-4′-methyl-2-(1-methyl-1H-1,2,3-triazol-4-yl)-4′,5′-dihydrospiro[piperidine-4,7′-thieno[2,3-c]pyran]-4′-ol (38) [DIAST-2] (6.9 mg, 29%) was afforded as a white foam. ¹H NMR (300 MHz, Chloroform-d) δ 7.42 (s, 1H), 6.86 (s, 1H), 4.41 (dd, J=11.6, 2.6 Hz, 1H), 4.07 (s, 3H), 3.78 (s, 2H), 3.21 (td, J=12.4, 2.6 Hz, 1H), 3.11-2.97 (m, 1H), 2.38-2.23 (m, 2H), 2.14 (dd, J=14.1, 2.6 Hz, 1H), 1.92 (dd, J=13.4, 11.6 Hz, 1H), 1.77-1.63 (m, 1H), 1.45 (s, 3H). ESI-MS m/z 355.07 [M+H]⁺.

### Compounds 39, 40, and 41

### (2′S,7S)-2-chloro-4-(difluoromethyl)-2′-(1-methyltriazol-4-yl)spiro[5H-thieno[2,3-c]pyran-7,4′-piperidine]-4-ol (39)[DIAST-1] and (40)[DIAST-2) and (2S,4S)-2′-chloro-2-(1-methyltriazol-4-yl)spiro[piperidine-4,7′-thieno[2,3-c]pyran]-4′-one (41)

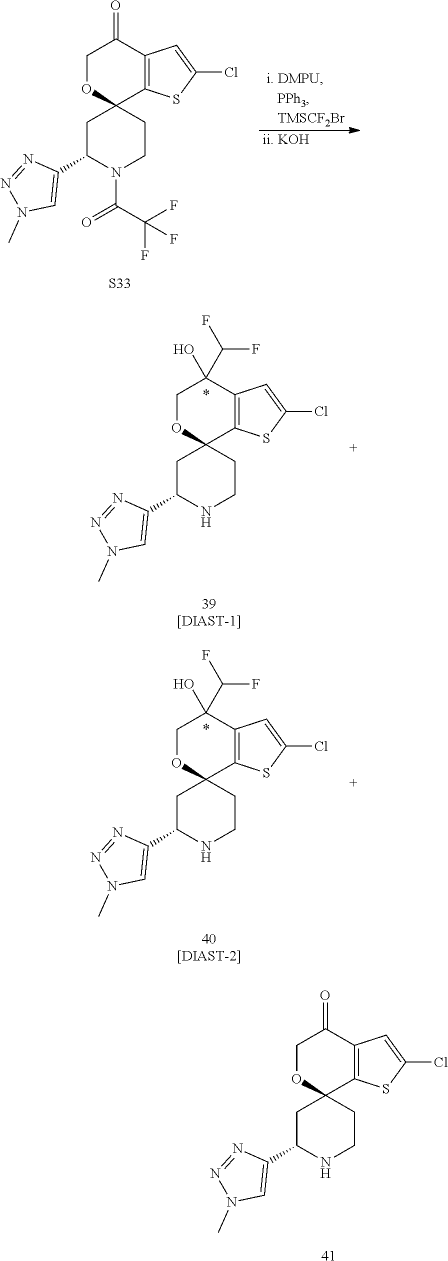

### Preparation of (2'S, 7S)-2-chloro-4-(difluoromethyl)-2′-(1-methyltriazol-4-yl)spiro[5H-thieno[2,3-c]pyran-7,4′-piperidine]-4-ol (39)[DIAST-1] and (40)[DIAST-2) and (2S,4S)-2′-chloro-2-(1-methyltriazol-4-yl)spiro[piperidine-4,7′-thieno[2,3-c]pyran]-4′-one (41)

In a small microwave vial, (2S,4S)-2′-chloro-2-(1-methyltriazol-4-yl)-1-(2,2,2-trifluoroacetyl)spiro[piperidine-4,7′-thieno[2,3-c]pyran]-4′-one S33 (30 mg, 0.06565 mmol) and PPh3 (21 mg, 0.08007 mmol) in MeCN (500 μL) at room temperature was added DMPU (18 μL, 0.1494 mmol) followed by [bromo(difluoro)methyl]-trimethyl-silane (19 μL). Resulting solution was heated to 60° C. overnight. The reaction was cooled to room temperature and treated with KOH (300 μL of 1 M, 0.3000 mmol) and after 10 min deprotected product was formed. The reaction was diluted with DCM and water and the organics collected via a phase separator and concentrated via rotovap. Purification by normal phase chromatography (Gradient: 0-15% MeOH in DCM) afforded the diastereomers as two separate peaks. Peak A afforded (2'S,7S)-2-chloro-4-(difluoromethyl)-2′-(1-methyltriazol-4-yl)spiro[5H-thieno[2,3-c]pyran-7,4′-piperidine]-4-ol 39 [DIAST-1] (5.3 mg, 19%) as a pale orange foam. ¹H NMR (400 MHz, Methanol-d₄) δ 7.82 (s, 1H), 6.98 (d, J=1.0 Hz, 1H), 5.95 (t, J=55.3 Hz, 1H), 4.27 (dd, J=11.8, 2.7 Hz, 1H), 4.08 (s, 4H), 3.80 (dd, J=12.2, 2.9 Hz, 1H), 3.24-3.14 (m, 1H), 3.01 (ddd, J=12.7, 4.7, 2.0 Hz, 1H), 2.52 (dt, J=13.9, 2.6 Hz, 1H), 2.02 (dd, J=13.9, 2.6 Hz, 1H), 1.91-1.76 (m, 2H). LCMS m/z 391.07 [M+H]⁺.

Peak B afforded (2'S,7S)-2-chloro-4-(difluoromethyl)-2′-(1-methyltriazol-4-yl)spiro[5H-thieno[2,3-c]pyran-7,4′-piperidine]-4-ol 40 [DIAST-2] (5.2 mg, 20%) as a semi-solid. ¹H NMR (400 MHz, Methanol-d₄) δ 7.81 (s, 1H), 6.97 (d, J=1.1 Hz, 1H), 5.95 (t, J=55.2 Hz, 1H), 4.29 (dd, J=11.8, 2.5 Hz, 1H), 4.10 (d, J=12.3 Hz, 1H), 4.08 (s, 3H), 3.77 (ddd, J=12.2, 3.3, 1.8 Hz, 1H), 3.17 (td, J=12.6, 2.6 Hz, 1H), 3.05-2.98 (m, 1H), 2.32-2.20 (m, 2H), 1.91 (dd, J=13.5, 11.8 Hz, 1H), 1.76 (ddd, J=14.0, 12.6, 4.7 Hz, 1H). ¹⁹F NMR (376 MHz, Methanol-d₄) δ −131.30 (d, J=281.2 Hz), −136.94 (d, J=281.2 Hz). LCMS m/z 391.07 [M+H]⁺.

Since the reaction had not gone to completion, some impure de-protected starting material was isolated. Purification by reversed-phase chromatography (Gradient: 0-100% MeCN in water with 0.1% TFA) afforded (2S,4S)-2′-chloro-2-(1-methyltriazol-4-yl)spiro[piperidine-4,7′-thieno[2,3-c]pyran]-4′-one 41 as a trifluoroacetate salt. LCMS m/z 339.21 [M+H]⁺.

### Compound 42

### (2'S, 7S)-2-chloro-4-methoxy-2′-(1-methyltriazol-4-yl)spiro[4,5-dihydrothieno[2,3-c]pyran-7,4′-piperidine]-1′-carboxylate (42)

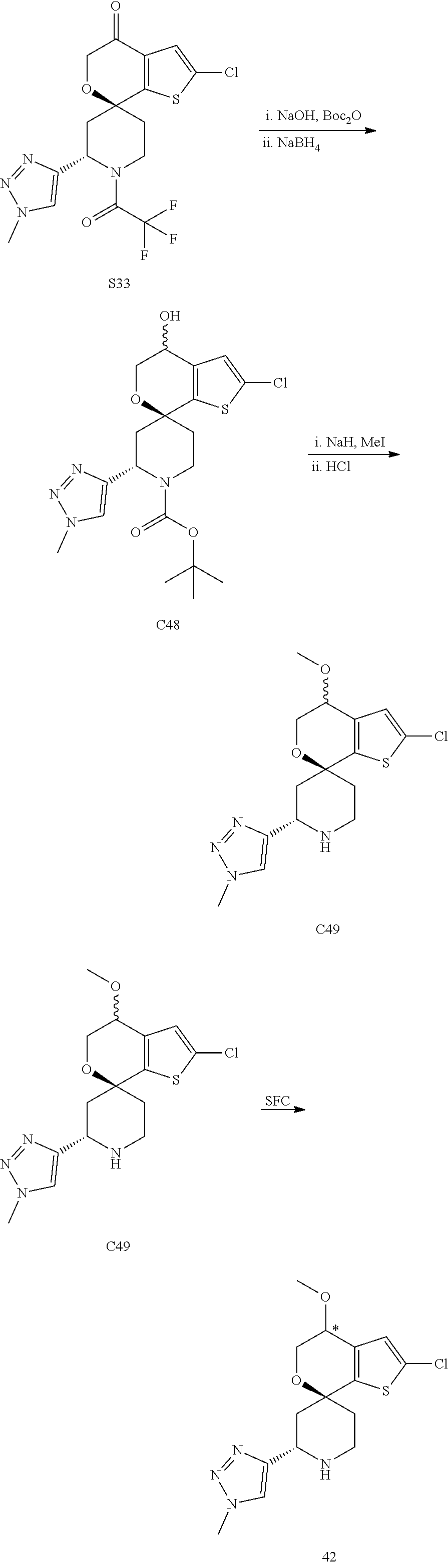

### Step 1. Synthesis of tert-butyl (2'S, 7S)-2-chloro-4-hydroxy-2′-(1-methyltriazol-4-yl)spiro[4,5-dihydrothieno[2,3-c]pyran-7,4′-piperidine]-1′-carboxylate (C48)

(2S,4S)-2′-chloro-2-(1-methyltriazol-4-yl)-1-(2,2,2-trifluoroacetyl)spiro[piperidine-4,7′-thieno[2,3-c]pyran]-4′-one S33 (80 mg, 0.1751 mmol) in MeOH (1 mL) was treated with NaOH (500 μL of 2 M, 1.000 mmol). After 20 min at room temperature an LCMS showed complete consumption of product and formation of intermediate 2. The reaction was diluted with DCM (1.5 mL) and treated with Boc₂O (75 mg, 0.3436 mmol) and stirred overnight at room temperature. The reaction was diluted with water and extracted with DCM (2×) through a phase separator. The organics were concentrated in vacuo and subsequently brought up in MeOH (1 mL) and treated with NaBH₄ (20 mg, 0.5286 mmol). After 10 min the reaction was quenched with water, diluted with DCM, and extracted (3×) through a phase separator. The organics were concentrated. Purification by normal phase chromatography (Gradient: 0-100% EtOAc in Heptane) yielded tert-butyl (2'S,7S)-2-chloro-4-hydroxy-2′-(1-methyltriazol-4-yl)spiro[4,5-dihydrothieno[2,3-c]pyran-7,4′-piperidine]-1′-carboxylate C48 (43 mg, 52%). LCMS m/z 441.12 [M+H]⁺.

### Step 2. Synthesis of (2'S,7S)-2-chloro-4-methoxy-2′-(1-methyltriazol-4-yl)spiro[4,5-dihydrothieno[2,3-c]pyran-7,4′-piperidine]-1′-carboxylate (C49)

Tert-butyl (2'S,7S)-2-chloro-4-hydroxy-2′-(1-methyltriazol-4-yl)spiro[4,5-dihydrothieno[2,3-c]pyran-7,4′-piperidine]-1′-carboxylate C48 (43 mg, 0.09041 mmol) in 2-MeTHF (1.5 mL) was treated with NaH (12 mg of 60% w/w in mineral oil, 0.3000 mmol) and stirred for 30 min. MeI (20 μL, 0.3213 mmol) was then added. After 2.5 hours the reaction was quenched with water, diluted with DCM, and extracted (2×) through a phase separator. The organic layer was concentrated via rotovap, brought up in DCM (1 mL) and treated with HCl (300 μL of 4 M in dioxane, 1.200 mmol). After 1.5 hours the reaction was basified with 2 M NaOH and extracted with DCM (2×) through a phase separator. The organics were concentrated in vacuo and purification via normal phase chromatography (Gradient: 0-15% MeOH in DCM) afforded (2'S,7S)-2-chloro-4-methoxy-2′-(1-methyltriazol-4-yl)spiro[4,5-dihydrothieno[2,3-c]pyran-7,4′-piperidine] C49 (25 mg, 78%) as a mixture of diastereomers. LCMS m/z 355.03 [M+H]⁺.

### Step 3. Synthesis of(2'S,7S)-2-chloro-4-methoxy-2′-(1-methyltriazol-4-yl)spiro[4,5-dihydrothieno[2,3-c]pyran-7,4′-piperidine]-1′-carboxylate (42)

(2'S,7S)-2-chloro-4-methoxy-2′-(1-methyltriazol-4-yl)spiro[4,5-dihydrothieno[2,3-c]pyran-7,4′-piperidine] C49 (25 mg) was separated into constituent enantiomers by chiral SFC separation. Column: Daicel Chiralpak® AD-H, 10×250 mm; Mobile Phase: 40% Ethanol (5 mM ammonia), 60% carbon dioxide. Peak B afforded (2'S,7S)-2-chloro-4-methoxy-2′-(1-methyltriazol-4-yl)spiro[4,5-dihydrothieno[2,3-c]pyran-7,4′-piperidine]42 (9.9 mg, 59%) as a pale yellow film. ¹H NMR (300 MHz, Chloroform-d) δ 7.38 (s, 1H), 6.79 (s, 1H), 4.40 (dd, J=11.6, 2.7 Hz, 1H), 4.16-3.91 (m, 6H), 3.43 (s, 3H), 3.19 (td, J=12.5, 2.7 Hz, 1H), 3.08-2.97 (m, 1H), 2.33 (dt, J=13.4, 2.7 Hz, 1H), 2.10 (dd, J=14.1, 2.6 Hz, 1H), 1.93 (dd, J=13.4, 11.7 Hz, 1H), 1.69 (ddd, J=13.9, 12.5, 4.7 Hz, 1H). LCMS m/z 355.03 [M+H]⁺.

### Compound 43

### (2'S, 7S)-2-chloro-4,4-dideuterio-2′-(1-methyltriazol-4-yl)spiro[5H-thieno[2,3-c]pyran-7,4′-piperidine] (43)

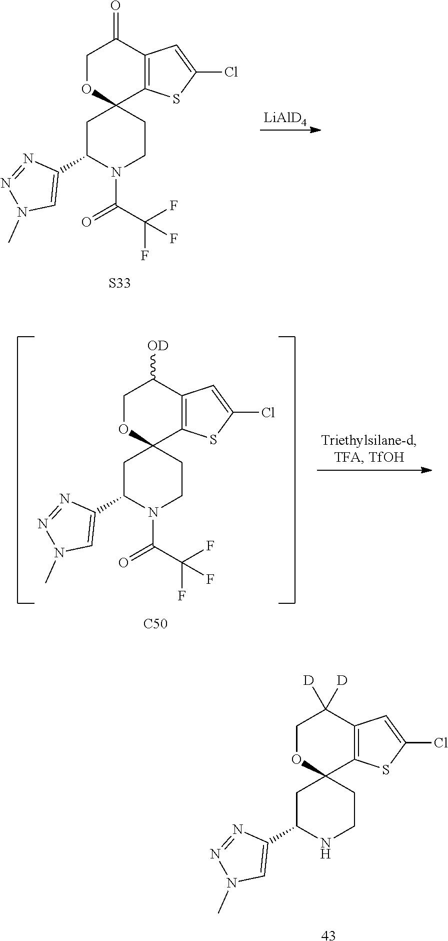

### Step 1. Synthesis of (2'S,7S)-2-chloro-4-deuterio-2′-(1-methyltriazol-4-yl)spiro[5H-thieno[2,3-c]pyran-7,4′-piperidine]-4-ol) (C50)

To an oven dried vial under argon was added LiAlD₄ (16.9 mg, 0.4026 mmol) and diethyl ether (1.5 mL). The solution was cooled to 0° C. and (2S,4S)-2′-chloro-2-(1-methyltriazol-4-yl)-1-(2,2,2-trifluoroacetyl)spiro[piperidine-4,7′-thieno[2,3-c]pyran]-4′-one S33 (32 mg, 0.06476 mmol) was added as a solution in diethyl ether (1.5 mL) and THF (1 mL). The reaction was warmed to room temperature and stirred for 30 minutes before it was quenched carefully with H₂O (10 mL) and pH adjusted with 2 M NaOH. The mixture was extracted with DCM (3×10 mL), passed through a phase separator, and concentrated to give crude C50.

### Step 2. Synthesis of (2'S, 7S)-2-chloro-4,4-dideuterio-2′-(1-methyltriazol-4-yl)spiro[5H-thieno[2,3-c]pyran-7,4′-piperidine] (43)

Crude C50 in CDCl₃ (1 mL) was treated with deuterio(triethyl)silane (70 μL, 0.4399 mmol) and TFA (330 μL, 4.283 mmol) and stirred for 45 minutes. TfOH (30 μL, 0.3390 mmol) was added and the reaction was stirred at room temperature for 60 minutes at which point it was quenched into water and DCM. The pH of the aqueous layer was adjusted to >10 with 2 M NaOH and extracted with DCM (3×). The organics were combined, dried over sodium sulfate, and concentrated. Purification by reversed-phase HPLC (Method: C18 Waters Sunfire column (30×150 mm, 5 micron). Gradient: MeCN in H₂O with 5 mM HCl) afforded (2'S,7S)-2-chloro-4,4-dideuterio-2′-(1-methyltriazol-4-yl)spiro[5H-thieno[2,3-c]pyran-7,4′-piperidine]43 (19.7 mg, 82%) as the hydrochloride salt. ¹H NMR (400 MHz, Methanol-d₄) δ 8.13 (s, 1H), 6.76 (s, 1H), 4.87 (dd, J=12.4, 3.2 Hz, 1H), 4.13 (s, 3H), 4.02 (d, J=2.4 Hz, 2H), 3.56 (td, J=13.1, 3.2 Hz, 1H), 3.42 (ddd, J=12.9, 4.7, 2.1 Hz, 1H), 2.55 (dt, J=14.6, 2.9 Hz, 1H), 2.41 (dd, J=14.7, 12.5 Hz, 1H), 2.37-2.29 (m, 1H), 2.15 (ddd, J=14.9, 13.4, 4.7 Hz, 1H). LCMS m/z 327.24 [M+H]⁺.

### Preparation S34

### 1-[2-bromo-2′-(1-methyltriazol-4-yl)spiro[4,5-dihydrothieno[2,3-c]pyran-7,4′-piperidine]-1′-yl]-2,2,2-trifluoro-ethanone (S34)

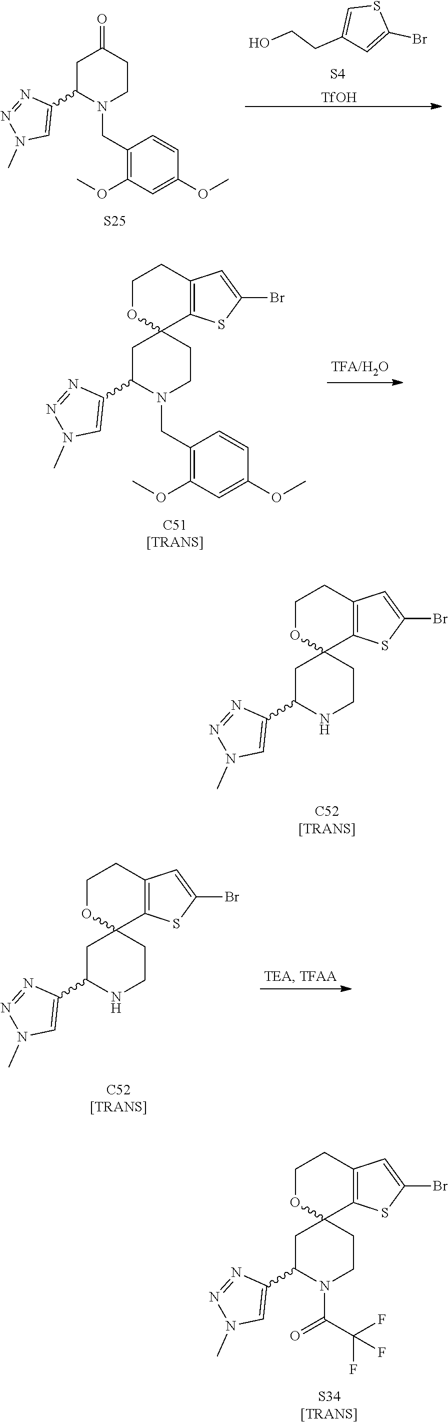

### Step 1. Synthesis of 2-bromo-1′-[(2,4-dimethoxyphenyl)methyl]-2′-(1-methyltriazol-4-yl)spiro[4,5-dihydrothieno[2,3-c]pyran-7,4′-piperidine](C51)

This compound was made following similar conditions to Step 1 of Compound 2 but using thiophene ethanol S4. The major product afforded 2-bromo-1′-[(2,4-dimethoxyphenyl)methyl]-2′-(1-methyltriazol-4-yl)spiro[4,5-dihydrothieno[2,3-c]pyran-7,4′-piperidine] (1.27 g, 77%) as a pair of enantiomers with assumed trans stereochemistry. LCMS m/z 519.2 [M+H]⁺.

### Step 2. Synthesis of 2-bromo-2′-(1-methyltriazol-4-yl)spiro[4,5-dihydrothieno[2,3-c]pyran-7,4′-piperidine] (C52)

This compound was made following similar conditions to Step 2 of Compound 2 using 2-bromo-1′-[(2,4-dimethoxyphenyl)methyl]-2′-(1-methyltriazol-4-yl)spiro[4,5-dihydrothieno[2,3-c]pyran-7,4′-piperidine] C51 (500 mg, 0.8449 mmol) as starting material. 2-bromo-2′-(1-methyltriazol-4-yl)spiro[4,5-dihydrothieno[2,3-c]pyran-7,4′-piperidine] C52 (312 mg) was afforded. LCMS m/z 369.07 [M+H]⁺.

### Step 3. 1-[2-bromo-2′-(1-methyltriazol-4-yl)spiro[4,5-dihydrothieno[2,3-c]pyran-7,4′-piperidine]-1′-yl]-2,2,2-trifluoro-ethanone (S34)

To a solution of 2-bromo-2′-(1-methyltriazol-4-yl)spiro[4,5-dihydrothieno[2,3-c]pyran-7,4′-piperidine] C52 (312 mg) in DCM (10 mL) was added TEA (383 mg, 3.785 mmol) and the solution was cooled to 0° C. TFAA (238 mg, 1.133 mmol) was added and the reaction was warmed to room temperature. After 1 hour the reaction mixture was diluted with sat. sodium bicarbonate and DCM. The organics were separated via a phase separator and concentrated via rotovap. Purification by normal phase chromatography (Gradient: 0-60% EtOAc in Heptane) yielded 1-[2-bromo-2′-(1-methyltriazol-4-yl)spiro[4,5-dihydrothieno[2,3-c]pyran-7,4′-piperidine]-1′-yl]-2,2,2-trifluoro-ethanone S34 (240 mg, 58%). ¹H NMR (400 MHz, Chloroform-d) δ 7.64 (d, J=17.1 Hz, 1H), 6.75 (s, 1H), 5.55 (d, J=9.8 Hz, 1H), 4.10 (s, 3H), 3.99-3.74 (m, 4H), 2.98 (t, J=13.1 Hz, 1H), 2.70-2.36 (m, 4H), 2.10 (d, J=19.5 Hz, 1H). LCMS m/z 465.15 [M+H]⁺.

### Preparation S35

### 1-[2-bromo-4-hydroxy-2′-(1-methyltriazol-4-yl)spiro[4,5-dihydrothieno[2,3-c]pyran-7,4′-piperidine-1′-yl]-2,2,2-trifluoro-ethanone (S35)

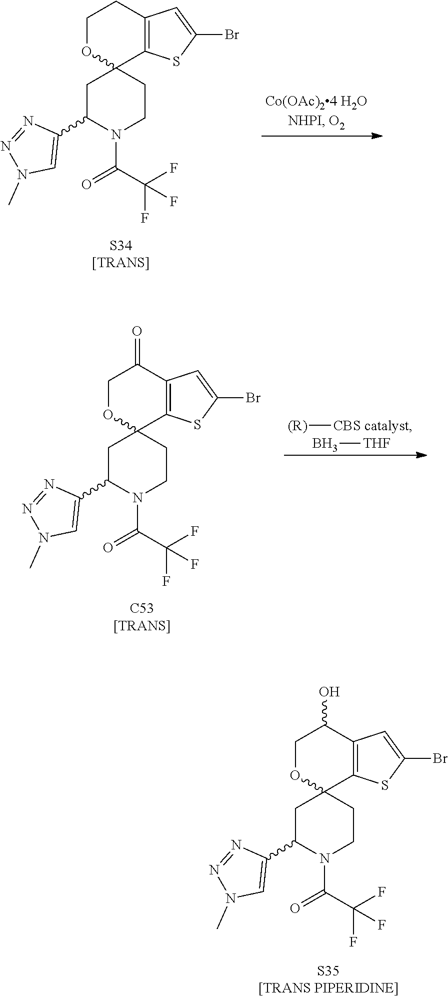

### Step 1. Synthesis of 2′-bromo-2-(1-methyltriazol-4-yl)-1-(2,2,2-trifluoroacetyl)spiro[piperidine-4,7′-thieno[2,3-c]pyran]-4′-one (C53)

A solution of 1-[2-bromo-2′-(1-methyltriazol-4-yl)spiro[4,5-dihydrothieno[2,3-c]pyran-7,4′-piperidine]-1′-yl]-2,2,2-trifluoro-ethanone S34 (240 mg, 0.5040 mmol) in MeCN (9.7 mL) was treated with cobalt(II) acetate tetrahydrate (13 mg, 0.05219 mmol) and N-hydroxyphthalimide (38 mg, 0.2329 mmol). The reaction was purged and evacuated with oxygen (3×) and heated to 45° C. under an oxygen balloon. After 8 hours the reaction was diluted with water and partitioned with DCM. The organics were collected via filtration through a phase separator and then concentrated via rotovap. Purification by silica gel chromatography (Gradient: 0-60% EtOAc in heptane) yielded the product 2′-bromo-2-(1-methyltriazol-4-yl)-1-(2,2,2-trifluoroacetyl)spiro[piperidine-4,7′-thieno[2,3-c]pyran]-4′-one C53 (130 mg, 52%). ¹H NMR (400 MHz, Chloroform-d) δ 7.67 (s, 1H), 7.37 (s, 1H), 5.57 (t, J=9.1 Hz, 1H), 4.18-3.86 (m, 6H), 3.17 (dd, J=14.6, 11.0 Hz, 1H), 2.75-2.57 (m, 2H), 2.15 (d, J=14.2 Hz, 1H). ¹⁹F NMR (376 MHz, Chloroform-d) 6-69.96. LCMS m/z 479.06 [M+H]⁺.

### Step 2. Synthesis of 1-[2-bromo-4-hydroxy-2′-(1-methyltriazol-4-yl)spiro[4,5-dihydrothieno[2,3-c]pyran-7,4′-piperidine]-1′-yl]-2,2,2-trifluoro-ethanone (S35)

To a flask with MTBE (1.67 mL) at 0° C. under N₂ was added (R)-(+)-2-Methyl-CBS-oxazaborolidine solution (50 μL of 1 M in toluene, 0.0500 mmol) followed by borane tetrahydrofuran (506 μL of 1 M in THF, 0.5060 mmol) added dropwise. Then a solution of 2′-bromo-2-(1-methyltriazol-4-yl)-1-(2,2,2-trifluoroacetyl)spiro[piperidine-4,7′-thieno[2,3-c]pyran]-4′-one C53 (125 mg, 0.2530 mmol) in MTBE (850 μL) was added dropwise. The reaction was stirred for 15 min at 0° C. and then quenched slowly with 1 M aq. HCl. The resulting mixture was warmed to room temperature and stirred overnight. The organic layer was separated and washed water and brine, dried over sodium sulfate, and concentrated. Purification by silica gel chromatography (Gradient: 0-60% EtOAc in heptane) yielded the product 1-[2-bromo-4-hydroxy-2′-(1-methyltriazol-4-yl)spiro[4,5-dihydrothieno[2,3-c]pyran-7,4′-piperidine]-1′-yl]-2,2,2-trifluoro-ethanone S35 (84 mg, 69%) as a mixture of stereoisomers. ¹H NMR (400 MHz, Chloroform-d) δ 7.61 (s, 1H), 6.98 (d, J=6.0 Hz, 1H), 5.67-5.36 (m, 1H), 4.48 (dq, J=9.3, 3.1 Hz, 1H), 4.08 (d, J=1.9 Hz, 3H), 4.01-3.75 (m, 4H), 2.98 (dt, J=57.0, 13.0 Hz, 1H), 2.66-2.35 (m, 2H), 2.19 (dd, J=8.9, 5.0 Hz, 1H), 1.96 (d, J=14.9 Hz, 1H). LCMS m/z 481.08 [M+H]⁺.

### Compounds 44 and 45

### 2-bromo-2′-(1-methyltriazol-4-yl)spiro[4,5-dihydrothieno[2,3-c]pyran-7,4′-piperidine]-4-ol (44) and (45)

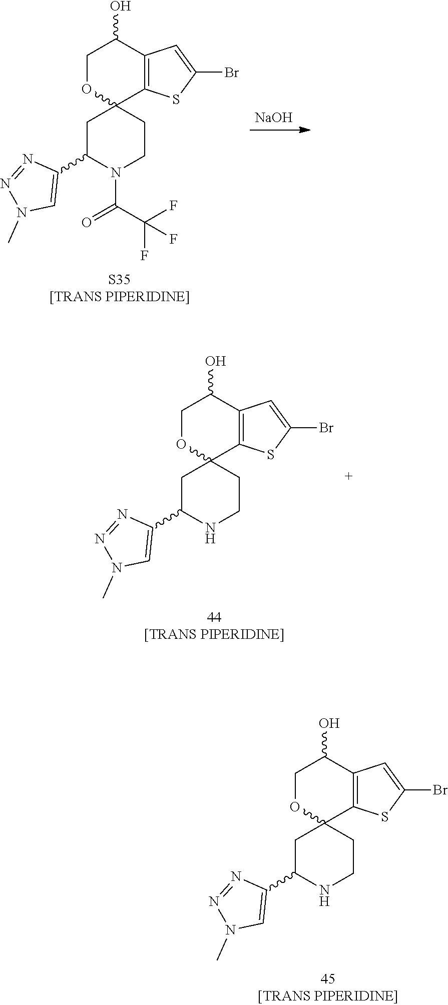 [SMILES: Cn1cc(C2CC3(CCN2C(=O)C(F)(F)F)OCC(O)c2cc(Br)sc23)nn1 ; "NaOH" ; Cn1cc(C2CC3(CCN2)OCC(O)c2cc(Br)sc23)nn1 ; Cn1cc(C2CC3(CCN2)OCC(O)c2cc(Br)sc23)nn1]

### Synthesis of 2-bromo-2′-(1-methyltriazol-4-yl)spiro[4,5-dihydrothieno[2,3-c]pyran-7,4′-piperidine]-4-ol (44) and (45)

S35 was purified to afford two product peaks and each were taken separately into DCM (700 μL) and treated with NaOH (127 μL of 2 M, 0.2540 mmol). After stirring for 30 minutes each the organics were collected through a phase separator and dried to give 2-bromo-2′-(1-methyltriazol-4-yl)spiro[4,5-dihydrothieno[2,3-c]pyran-7,4′-piperidine]-4-ol 44 (3.6 mg, 54%) TH NMR (400 MHz, DMSO-d₆) δ 7.89 (s, 1H), 7.07 (s, 1H), 5.42 (d, J=6.6 Hz, 1H), 4.43 (d, J=5.1 Hz, 1H), 4.14-3.86 (m, 5H), 3.62 (dd, J=11.7, 5.6 Hz, 1H), 3.08-2.76 (m, 2H), 2.17 (d, J=13.4 Hz, 1H), 2.02 (d, J=13.5 Hz, 1H), 1.76-1.54 (m, 2H). LCMS m/z 385.09 [M+H]⁺; and 2-bromo-2′-(1-methyltriazol-4-yl)spiro[4,5-dihydrothieno[2,3-c]pyran-7,4′-piperidine]-4-ol 45 (1.8 mg, 60%). ¹H NMR (400 MHz, DMSO-d₆) δ 7.90 (s, 1H), 7.07 (s, 1H), 5.40 (s, 1H), 4.41 (s, 1H), 4.18-3.79 (m, 5H), 3.64 (dd, J=11.8, 5.1 Hz, 1H), 3.07-2.73 (m, 2H), 2.29 (d, J=13.3 Hz, 1H), 1.92 (d, J=13.3 Hz, 1H), 1.76-1.46 (m, 2H). LCMS m/z 385.09 [M+H]⁺.

### Compound 46

### 2-(3,3-difluorocyclobutyl)-2′-(1-methyltriazol-4-yl)spiro[4,5-dihydrothieno[2,3-c]pyran-7,4′-piperidine 1 (46)

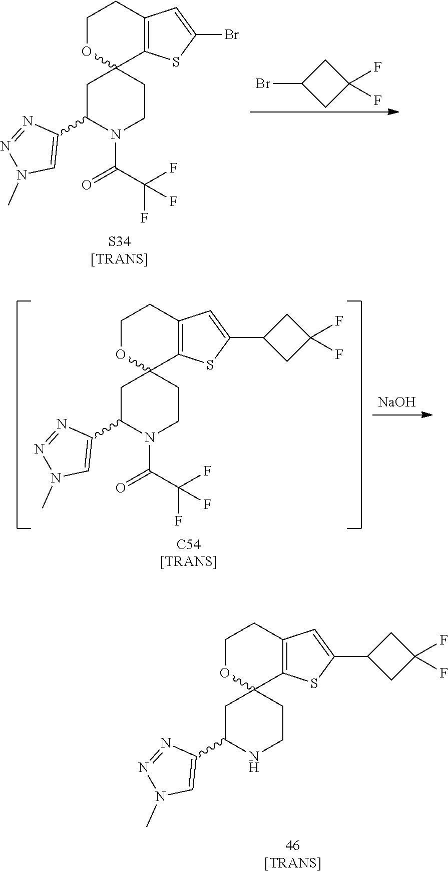

### Synthesis of 2-(3,3-difluorocyclobutyl)-2′-(1I-methyltriazol-4-yl)spiro[4,5-dihydrothieno[2,3-c]pyran-7,4′-piperidine] (46)

To vial was added Ir[df(CF3)ppy]2(dtbbpy)PF₆ (2 mg, 0.001981 mmol), NiCl₂ glyme (4 mg, 0.01820 mmol), and 4-tert-butyl-2-(4-tert-butyl-2-pyridyl)pyridine (5 mg, 0.01863 mmol) as solids under an inert atmosphere. A solution of 1-[2-bromo-2′-(1-methyltriazol-4-yl)spiro[4,5-dihydrothieno[2,3-c]pyran-7,4′-piperidine]-1′-yl]-2,2,2-trifluoro-ethanone S34 (80.68 mg, 0.1578 mmol) in DME (1 mL) was added, followed sequentially by a solution of bis(trimethylsilyl)silyl-trimethyl-silane (45 mg, 0.1810 mmol), 2,6-dimethylpyridine (38 mg, 0.3546 mmol), and 3-bromo-1,1-difluoro-cyclobutane (207.5 μL, 1.578 mmol) in DME (1 mL). The vial was sealed and irradiated in a Sigma SynLED photoreactor overnight. The reaction vial was unsealed, diluted with water (2 mL) and DCM (2 mL), and stirred for several minutes. The biphasic mixtures were passed through a parallel hydrophobic filter plate. The organic layers were evaporated to afford crude C54. To this was added methanol (1.050 mL) and NaOH (283.2 μL of 6 M, 1.699 mmol). The resulting mixture was stirred at 55° C. for 20 minutes. The reaction mixture was evaporated via Genevac at 40° C. Water (2 mL) and DCM (2 mL) were added, and the mixtures were passed through a phase separator. The organic layer was concentrated in vacuo. Purification by reversed-phase HPLC (Method: C18 Waters Sunfire column (30×150 mm, 5 micron). Gradient: MeCN in H₂O with 0.1% trifluoroacetic acid) afforded 2-(3,3-difluorocyclobutyl)-2′-(1-methyltriazol-4-yl)spiro[4,5-dihydrothieno[2,3-c]pyran-7,4′-piperidine]46 (8.6 mg, 10%) as a trifluoroacetate salt. ¹H NMR (400 MHz, Methanol-d₄) δ 8.04 (s, 1H), 6.70 (d, J=0.9 Hz, 1H), 4.86 (dd, J=12.5, 3.1 Hz, 1H), 4.12 (s, 3H), 4.09-3.93 (m, 2H), 3.66-3.49 (m, 2H), 3.40 (ddd, J=12.8, 4.6, 2.1 Hz, 1H), 3.16-2.87 (m, 2H), 2.76-2.01 (m, 8H). LCMS m/z 381.28 [M+H]⁺.

### Preparation S37

### tert-butyl 2-bromo-2′-phenyl-spiro[4,5-dihydrothieno[2,3-c]pyran-7,4′-piperidine]-1′-carboxylate (S36)

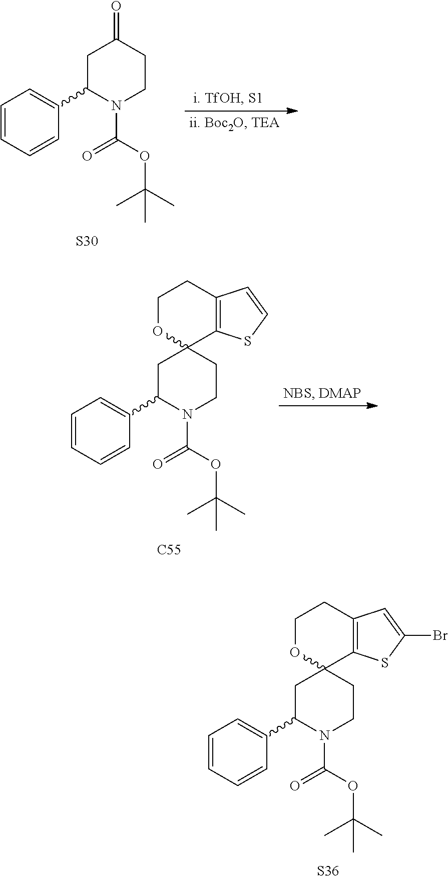 [SMILES: CC(C)(C)OC(=O)N1CCC(=O)CC1c1ccccc1 ; "S30" ; CC(C)(C)OC(=O)N1CCC2(CC1c1ccccc1)OCCc1ccsc12 ; "C55" ; CC(C)(C)OC(=O)N1CCC2(CC1c1ccccc1)OCCc1cc(Br)sc12 ; "S36"]

### Step 1. Synthesis of tert-butyl 2′-phenylspiro[4,5-dihydrothieno[2,3-c]pyran-7,4′-piperidine]-1′-carboxylate (C55)

To a solution of tert-butyl 4-oxo-2-phenyl-piperidine-1-carboxylate S30 (3 g, 10.90 mmol) and 2-(3-thienyl)ethanol S1 (2 g, 15.60 mmol) in dioxane (15 mL) at 0° C. was added triflic acid (2.95 mL, 33.32 mmol) dropwise. The reaction was allowed to warm to room temperature and stirred for three hours. The reaction mixture was concentrated via rotovap to afford crude 2′-phenylspiro[4,5-dihydrothieno[2,3-c]pyran-7,4′-piperidine] (3 g, 45%). LCMS m/z 285.96 [M+H]⁺. This material was brought up in ACN (50 mL) and treated with Boc₂O (1.5 g, 6.873 mmol) and TEA (1 g, 9.882 mmol) and the reaction was stirred overnight. The reaction mixture was then concentrated, diluted in EtOAc and washed with sat. sodium bicarbonate. The organic layer was washed with brine, dried over sodium sulfate and concentrated in vacuo. Purification by silica gel chromatography (Gradient: 0-100% EtOAc in Heptane) afforded tert-butyl 2′-phenylspiro[4,5-dihydrothieno[2,3-c]pyran-7,4′-piperidine]-1′-carboxylate C55 (2.5 g, 72%). LCMS m/z 386.14 [M+H]⁺.

### Step 2. Synthesis of tert-butyl 2-bromo-2′-phenyl-spiro[4,5-dihydrothieno[2,3-c]pyran-7,4′-piperidine]-1′-carboxylate (S36)

Tert-butyl 2′-phenylspiro[4,5-dihydrothieno[2,3-c]pyran-7,4′-piperidine]-1′-carboxylate C55 (600 mg, 1.556 mmol) was taken up in ACN (20 mL) and DMAP (20 mg, 0.1637 mmol) was added, followed by NBS (280 mg, 1.573 mmol). The reaction was stirred at room temperature overnight. The reaction mixture was quenched with sat. sodium bicarbonate and diluted with water and ethyl acetate. The organic layer was washed with water and brine, dried over sodium sulfate, and concentrated. Purification by silica gel chromatography (Gradient: 0-100% EtOAc in Heptane) yielded tert-butyl 2-bromo-2′-phenyl-spiro[4,5-dihydrothieno[2,3-c]pyran-7,4′-piperidine]-1′-carboxylate S36 (700 mg, 80%) as a mixture of stereoisomers. LCMS 463.94 [M+H]⁺.

### Compounds 47-50

Compounds 47-50 (see Table 4) were prepared by methods similar to Compound 46 with modifications obvious to someone skilled in the art based on the protecting group present. All compounds were isolated as trifluoroacetate salts. Alkyl halides were obtained from commercial sources.

TABLE 4
Structure and physicochemical data for compounds 47-50.

| | | Bromo | |
|---|---|---|---|
| | | thiophene and | |
| Compound | Product | Alkyl halide | ¹H NMR; LCMS m/z [M + H]⁺ |
| 47 | 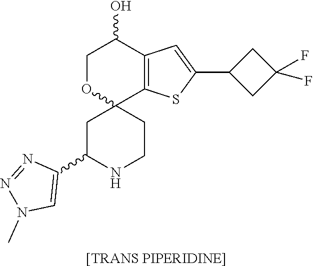 | 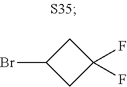 | LCMS m/z 397.27 [M + H]⁺ |
| 48 | 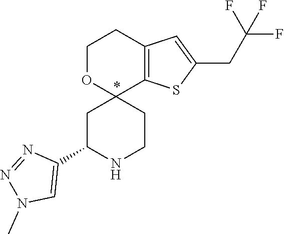 [SMILES: Cn1cc([C@@H]2CC3(CCN2)OCCc2cc(CC(F)(F)F)sc23)nn1] | 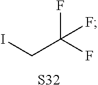 | LCMS m/z 373.29 [M + H]⁺ |
| 49 | 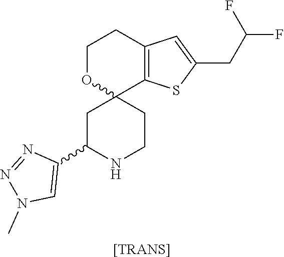 | 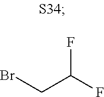 | ¹H NMR (400 MHz, Methanol- d₄) δ 8.04 (s, 1H), 6.73 (s, 1H), 5.99 (tt, J = 56.5, 4.2 Hz, 1H), 4.88-4.82 (m, 1H), 4.12 (s, 3H), 4.08-3.86 (m, 2H), 3.56 (td, J = 13.2, 3.2 Hz, 1H), 3.44- 3.25 (m, 3H), 2.70 (t, J = 5.5 Hz, 2H), 2.54 (dt, J = 14.7, 2.9 Hz, 1H), 2.44-2.28 (m, 2H), 2.13 (ddd, J = 14.9, 13.4, 4.7 Hz, 1H). LCMS m/z 355.32 [M + H]⁺ |
| 50 | 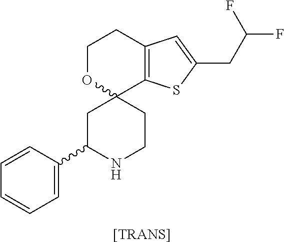 | 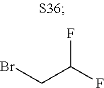 | ¹H NMR (300 MHz, Acetone- d₆) δ 7.74-7.55 (m, 2H), 7.51- 7.28 (m, 3H), 6.75 (s, 1H), 6.13 (tt, J = 56.5, 4.3 Hz, 1H), 4.72 (dd, J = 12.7, 2.9 Hz, 1H), 4.04 (td, J = 5.6, 2.3 Hz, 2H), 3.59 (td, J = 12.9, 3.3 Hz, 1H), 3.50- 3.30 (m, 3H), 2.75-2.59 (m, 3H), 2.51-2.18 (m, 3H). LCMS m/z 350.14 [M + H]⁺ |

### Preparation S37

### (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran] (S37)

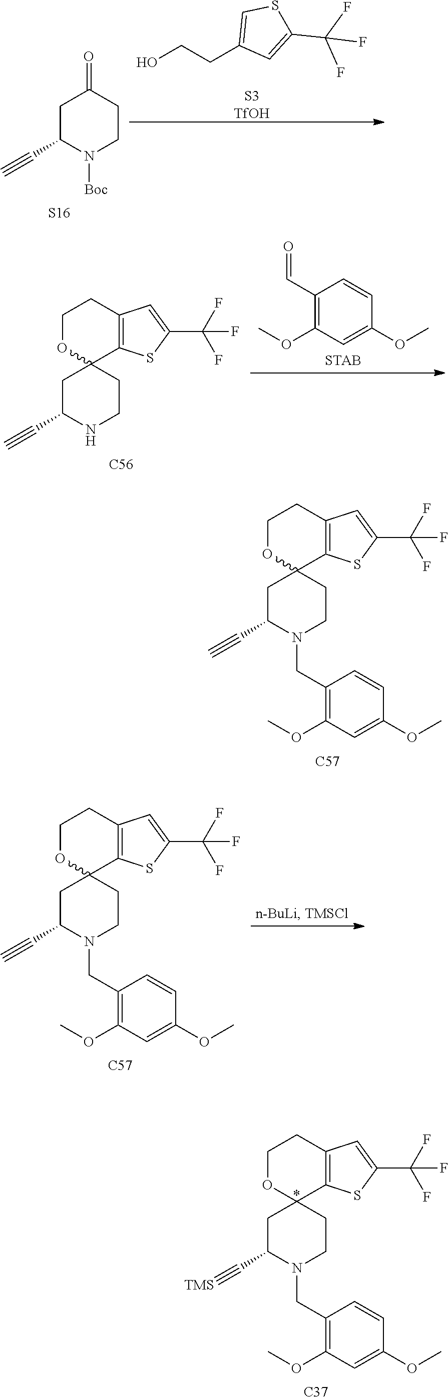

### Step 1. Synthesis of (2S)-2-ethynyl-2′-(trifluoromethyl)-4′,5′-dihydrospiro[piperidine-4,7′-thieno[2,3-c]pyran] (C56)

Tert-butyl (S)-2-ethynyl-4-oxopiperidine-1-carboxylate S16 (3.4 g, 15.23 mmol) and 2-[5-(trifluoromethyl)-3-thienyl]ethanol S3 (3.5 g, 17.30 mmol) were dissolved in dioxane (51 mL) and cooled to 0° C. Triflic acid (4 mL, 45.20 mmol) was added dropwise and the reaction was warmed to room temperature. After 6 hours the solution was diluted with DCM and quenched with 2 M Na₂CO₃. The mixture was extracted with DCM (3 vol. eq.), dried over sodium sulfate and concentrated in vacuo. Purification by silica gel chromatography (Gradient: 0-20% MeOH in DCM) yielded the product (2S)-2-ethynyl-2′-(trifluoromethyl)-4′,5′-dihydrospiro[piperidine-4,7′-thieno[2,3-c]pyran] C56 (3.8 g, 56%) as a mixture of stereoisomers. LCMS m/z 302.06 [M+H]⁺.

### Step 2. Synthesis of (2S)-1-(2,4-dimethoxybenzyl)-2-ethynyl-2′-(trifluoromethyl)-4′,5′-dihydrospiro[piperidine-4,7′-thieno[2,3-c]pyran] (C57)

To a solution of 2,4-dimethoxybenzaldehyde (2.75 g, 16.55 mmol) in DCE (43 mL) was added (2S)-2-ethynyl-2′-(trifluoromethyl)-4′,5′-dihydrospiro[piperidine-4,7′-thieno[2,3-c]pyran] C56 (3.7 g, 8.271 mmol) and sodium triacetoxyborohydride (5.2 g, 24.65 mmol). The mixture was stirred for 30 minutes and then quenched with sat. sodium bicarbonate. The solution was diluted with DCM and the organic layer collected through a phase separator. The solvent was removed in vacuo. Purification by silica gel chromatography (Gradient: 0-60% EtOAc in heptane) yielded (2S)-1-(2,4-dimethoxybenzyl)-2-ethynyl-2′-(trifluoromethyl)-4′,5′-dihydrospiro[piperidine-4,7′-thieno[2,3-c]pyran] C57 (3.4 g, 86%) as a mixture of diastereomers. LCMS m/z 452.14 [M+H]⁺.

### Step 3. Synthesis of (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran] (S37)

To a solution of (2S)-1-(2,4-dimethoxybenzyl)-2-ethynyl-2′-(trifluoromethyl)-4′,5′-dihydrospiro[piperidine-4,7′-thieno[2,3-c]pyran](3.4 g, 7.087 mmol) in THF (48 mL) at −78° C. under a nitrogen atmosphere was added n-BuLi (4.25 mL of 2.5 M in hexanes, 10.62 mmol). The reaction was stirred for 30 minutes at −78° C. and then TMSCl (7.8 mL of 1 M in THF, 7.800 mmol) was added. The reaction was allowed to warm to room temperature and after 30 minutes was quenched with sat. ammonium chloride solution and diluted with water. The mixture was extracted with DCM (3×) and the organic layer dried over sodium sulfate and dried in vacuo. Purification by silica gel chromatography (Gradient: 0-50% EtOAc in heptane) separated the two diastereomers. Peak B afforded the desired (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran] S37 (1.1 g, 59%). ¹H NMR (300 MHz, Chloroform-d) δ 7.38 (d, J=8.2 Hz, 1H), 7.09 (d, J=1.4 Hz, 1H), 6.54-6.43 (m, 2H), 4.29 (d, J=13.3 Hz, 1H), 3.88 (td, J=5.6, 1.9 Hz, 2H), 3.81 (d, J=5.4 Hz, 6H), 3.65 (d, J=13.3 Hz, 1H), 3.40 (dd, J=11.6, 2.8 Hz, 1H), 2.77 (dt, J=12.0, 3.6 Hz, 1H), 2.65 (dt, J=6.5, 3.3 Hz, 2H), 2.43-2.28 (m, 2H), 2.08 (dd, J=14.0, 11.6 Hz, 1H), 1.95-1.85 (m, 2H), 0.17 (s, 9H). ¹⁹F NMR (282 MHz, Chloroform-d) δ −55.26.

### Compound 51

### ((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)methanol (51)

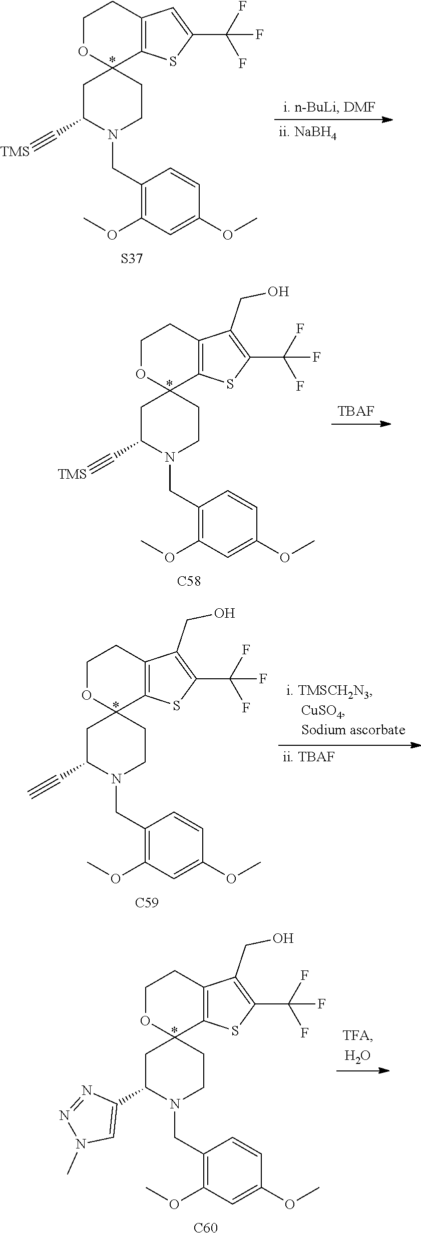

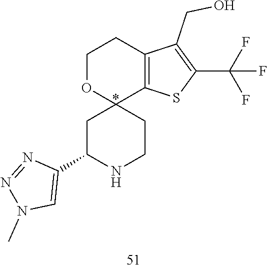 [SMILES: Cn1cc([C@@H]2CC3(CCN2)OCCc2c3sc(C(F)(F)F)c2CO)nn1 ; "51"]

### Step 1. Synthesis of ((2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran]-3′-yl)methanol (C58)

To a solution of (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran] S37 (350 mg, 0.6590 mmol) in THF (5 mL) at −78° C. under nitrogen was added n-BuLi (430 μL of 2.3 M in hexanes, 0.9890 mmol). The reaction was stirred for 30 minutes and then DMF (51 μL, 0.6587 mmol) was added. The reaction was slowly warmed to room temperature and then quenched with NH₄Cl and diluted with DCM. The organic layer was collected through a phase separator and dried give crude aldehyde (363 mg) which was immediately dissolved in MeOH (700 μL) and DCM (2.7 mL) and treated with sodium borohydride (264 μL, 6.594 mmol). After 15 minutes the reaction was quenched with sat. sodium bicarbonate solution, diluted with DCM, and the organics extracted through a phase separator. The volatiles were removed in vacuo. Purification by silica gel chromatography (Gradient: 0-70% EtOAc in heptane) yielded ((2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran]-3′-yl)methanol C58 (113 mg, 31%). ¹H NMR (300 MHz, Chloroform-d) δ 7.19 (s, 1H), 6.39-6.25 (m, 2H), 4.46 (d, J=3.3 Hz, 2H), 4.12 (d, J=13.3 Hz, 1H), 3.82-3.72 (m, 2H), 3.64 (d, J=5.2 Hz, 6H), 3.48 (d, J=13.3 Hz, 1H), 3.24 (dd, J=11.6, 2.8 Hz, 1H), 2.66-2.38 (m, 3H), 2.17 (td, J=12.4, 3.3 Hz, 2H), 2.01-1.84 (m, 1H), 1.83-1.62 (m, 2H), 0.00 (s, 9H). LCMS m/z 554.13 [M+H]⁺.

### Step 2. Synthesis of ((2S)-1-(2,4-dimethoxybenzyl)-2-ethynyl-2′-(trifluoromethyl)-4′,5′-dihydrospiro[piperidine-4,7′-thieno[2,3-c]pyran]-3′-yl)methanol (C59)

To a solution of ((2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran]-3′-yl)methanol C58 (115 mg, 0.2077 mmol) in THF (1.75 mL) was added TBAF (56 μL, 0.1900 mmol) at 0° C. The reaction was warmed to room temperature and after 1 hour was quenched with sat. sodium bicarbonate solution and diluted with DCM. The organic layer was collected through a phase separator and concentrated via rotovap. Purification by silica gel chromatography (Gradient: 0-70% EtOAc in heptane) yielded the product ((2S)-1-(2,4-dimethoxybenzyl)-2-ethynyl-2′-(trifluoromethyl)-4′,5′-dihydrospiro[piperidine-4,7′-thieno[2,3-c]pyran]-3′-yl)methanol C59 (95 mg, 92%). ¹H NMR (300 MHz, Chloroform-d) δ 7.39 (d, J=8.2 Hz, 1H), 6.67-6.36 (m, 2H), 4.66 (d, J=5.0 Hz, 2H), 4.40-4.10 (m, 1H), 4.07-3.34 (m, 10H), 2.85-2.52 (m, 3H), 2.52-2.24 (m, 3H), 2.24-1.76 (m, 3H). LCMS m/z 482.16 [M+H]⁺.

### Step 3. Synthesis of((2S)-1-(2,4-dimethoxybenzyl)-2-(1-methyl-1H-1,2,3-triazol-4-yl)-2′-(trifluoromethyl)-4′,5′-dihydrospiro[piperidine-4,7′-thieno[2,3-c]pyran]-3′-yl)methanol (C₆₀)

To a solution of ((2S)-1-(2,4-dimethoxybenzyl)-2-ethynyl-2′-(trifluoromethyl)-4′,5′-dihydrospiro[piperidine-4,7′-thieno[2,3-c]pyran]-3′-yl)methanol C59 (95 mg, 0.1973 mmol), copper(II) sulfate (15 mg, 0.09398 mmol), and sodium ascorbate (15 mg, 0.07533 mmol) in DMF (400 μL) was added azidomethyl(trimethyl)silane (124 μL of 1.5 M, 0.1860 mmol). The reaction was heated to 40° C. for 4 hours and then cooled to room temperature and continued to stir for another 48 hrs. The reaction was quenched with sat. sodium bicarbonate solution and DCM and the organic layer was collected through a phase separator. The solvent was removed in vacuo to give crude TMS protected intermediate, LCMS m/z 611.2 [M+H]⁺. The crude reaction mixture was dissolved in THF (950 μL) and TBAF (70 μL, 0.2375 mmol) was added at 0° C. The reaction was stirred for 3 hours at which point full conversion was observed. The reaction was quenched with sat. sodium bicarbonate solution, diluted with DCM, and passed through a phase separator. The organics were dried via rotovap and purification by silica gel chromatography (Gradient: 0-70% EtOAc in heptane) yielded the product ((2S)-1-(2,4-dimethoxybenzyl)-2-(1-methyl-1H-1,2,3-triazol-4-yl)-2′-(trifluoromethyl)-4′,5′-dihydrospiro[piperidine-4,7′-thieno[2,3-c]pyran]-3′-yl)methanol C₆₀ (52 mg, 46%). ¹H NMR (300 MHz, Chloroform-d) δ 7.56 (s, 1H), 7.25 (d, J=8.2 Hz, 1H), 6.54-6.30 (m, 2H), 4.65 (s, 2H), 4.05 (d, J=20.9 Hz, 6H), 3.78 (d, J=15.4 Hz, 6H), 3.67 (d, J=13.8 Hz, 1H), 3.21 (d, J=13.8 Hz, 1H), 2.87 (s, 1H), 2.75 (dt, J=7.2, 3.6 Hz, 2H), 2.58 (td, J=11.1, 5.1 Hz, 1H), 2.34 (d, J=13.9 Hz, 1H), 2.08-1.91 (m, 4H). LCMS m/z 539.24 [M+H]⁺.

### Step 4. Synthesis of ((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)methanol (51)

((2S)-1-(2,4-dimethoxybenzyl)-2-(1-methyl-1H-1,2,3-triazol-4-yl)-2′-(trifluoromethyl)-4′,5′-dihydrospiro[piperidine-4,7′-thieno[2,3-c]pyran]-3′-yl)methanol C₆₀ (45 mg, 0.08355 mmol) in a mixture of water (78 μL, 4.330 mmol) and TFA (227 μL, 2.946 mmol) was heated to 90° C. Upon completion, the reaction was cooled to room temperature, diluted with DCM, and then poured slowly into 6 M NaOH aq. solution. The organic layer was collected via a phase separator and dried in vacuo. Separated organic layer through a phase separator and blew off solvent. Purification by silica gel chromatography (Gradient: 0-20% MeOH in DCM) yielded the product ((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)methanol 51 (15.6 mg, 48%). ¹H NMR (300 MHz, Chloroform-d) δ 7.45 (s, 1H), 4.64 (d, J=1.4 Hz, 2H), 4.37 (dd, J=11.7, 2.6 Hz, 1H), 4.02 (d, J=17.2 Hz, 5H), 3.25 (td, J=12.4, 2.7 Hz, 1H), 3.00 (ddd, J=12.3, 4.9, 2.1 Hz, 1H), 2.75 (t, J=5.5 Hz, 2H), 2.36 (dt, J=13.6, 2.7 Hz, 1H), 2.04 (q, J=2.7 Hz, 1H), 1.95-1.68 (m, 2H). LCMS m/z 389.18 [M+H]⁺.

### Compounds 52 and 53

### 1-((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)-113-ethan-1-ol (52)[DIAST-1] and (53)[DIAST-2]

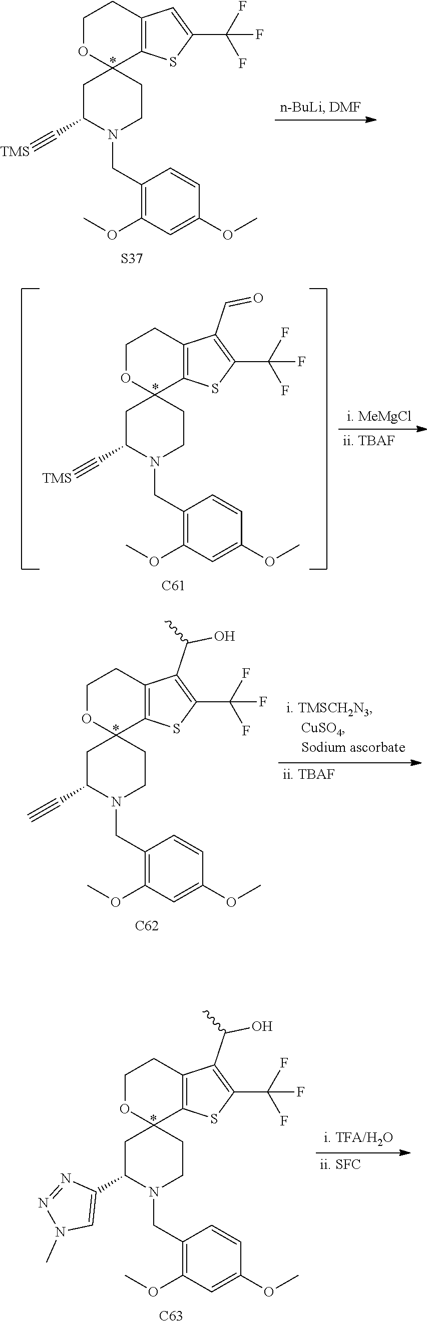

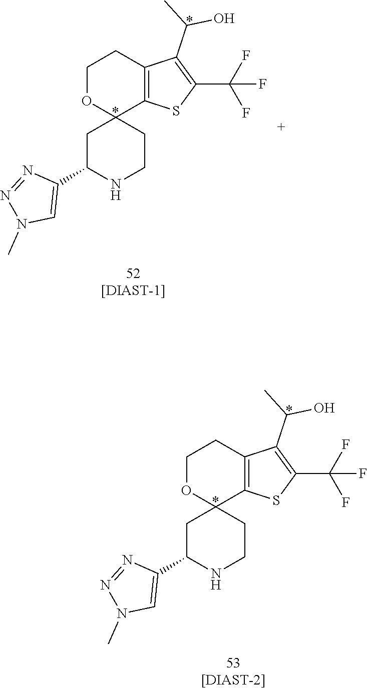

### Step 1. Synthesis of (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran]-3′-carbaldehyde (C61)

To a solution of 2-[1′-[(2,4-dimethoxyphenyl)methyl]-2-(trifluoromethyl)spiro[4,5-dihydrothieno[2,3-c]pyran-7,4′-piperidine]-2′-yl]ethynyl-trimethyl-silane (1.1 g, 2.078 mmol) in THF (15 mL) at −78° C. under N₂ was added n-BuLi (1.4 mL of 2.3 M in hexanes, 3.220 mmol). The reaction was continued stirring at −78° C. for 30 min and then DMF (240 μL, 3.100 mmol) was added. The reaction was warmed to room temperature over 30 min and then quenched with sat. ammonium chloride and diluted with DCM. The organic layer was collected through a phase separator and concentrated to give crude (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran]-3′-carbaldehyde C61 (460 mg, 40%) which was taken forward directly to the next step. LCMS m/z 552.1 [M+H]⁺.

### Step 2. Synthesis of]-((2S)-1-(2,4-dimethoxybenzyl)-2-ethynyl-2′-(trifluoromethyl)-4′,5′-dihydrospiro[piperidine-4,7′-thieno[2,3-c]pyran]-3′-yl)ethan-1-ol (C62)

Crude (2S)-1-(2,4-dimethoxybenzyl)-2′-(trifluoromethyl)-2-((trimethylsilyl)ethynyl)-4′,5′-dihydrospiro[piperidine-4,7′-thieno[2,3-c]pyran]-3′-carbaldehyde C61 (460 mg) was dissolved in THF (15 mL) and cooled to 0° C. Methylmagnesium chloride (1.22 mL of 3.4 M in THF, 4.148 mmol) was added. The reaction was stirred for 20 minutes and full conversion was observed. To this reaction mixture was added TBAF (4.1 mL of 1 M, 4.100 mmol). After two hours the reaction was quenched with sat. sodium bicarbonate solution and diluted with DCM. The organic layer was collected through a phase separator and the solvent removed in vacuo. Purification by silica gel chromatography (Gradient: 0-70% EtOAc in heptane) yielded the product 1-((2S)-1-(2,4-dimethoxybenzyl)-2-ethynyl-2′-(trifluoromethyl)-4′,5′-dihydrospiro[piperidine-4,7′-thieno[2,3-c]pyran]-3′-yl)ethan-1-ol C62 (345 mg, 83%). ¹H NMR (300 MHz, Chloroform-d) δ 7.39 (d, J=8.2 Hz, 1H), 6.66-6.39 (m, 2H), 5.27 (p, J=7.7, 6.9 Hz, 1H), 4.30 (d, J=13.5 Hz, 1H), 3.99-3.73 (m, 8H), 3.63 (d, J=13.5 Hz, 1H), 3.50-3.38 (m, 1H), 3.00 (dq, J=16.4, 5.5 Hz, 1H), 2.77 (dq, J=14.2, 5.8 Hz, 2H), 2.51-2.25 (m, 3H), 2.12 (ddd, J=14.1, 11.6, 8.8 Hz, 1H), 2.01-1.80 (m, 3H), 1.58-1.44 (m, 3H). LCMS m/z 496.07 [M+H]⁺.

### Step 3. Synthesis of 1-((2S)-1-(2,4-dimethoxybenzyl)-2-(1-methyl-1H-1,2,3-triazol-4-yl)-2′-(trifluoromethyl)-4′,5′-dihydrospiro[piperidine-4,7′-thieno[2,3-c]pyran]-3′-yl)ethan-1-ol (C63)

To a solution of 1-((2S)-1-(2,4-dimethoxybenzyl)-2-ethynyl-2′-(trifluoromethyl)-4′,5′-dihydrospiro[piperidine-4,7′-thieno[2,3-c]pyran]-3′-yl)ethan-1-ol C62 (340 mg, 0.6721 mmol), copper(II) sulfate (54 mg, 0.3383 mmol), and sodium ascorbate (67 mg, 0.3365 mmol) in DMF (1.4 mL) was added azidomethyl(trimethyl)silane (422 μL of 1.5 M, 0.6330 mmol). The reaction was heated to 40° C. for 4 hours and then cooled to room temperature and continued to stir for another 48 hrs. The reaction was quenched with sat. sodium bicarbonate solution and DCM and the organic layer was collected through a phase separator. The solvent was removed in vacuo to give crude TMS protected intermediate, LCMS m/z 625.11 [M+H]⁺. The crude reaction mixture was dissolved in THF (3.3 mL) and TBAF (238 μL, 0.8070 mmol) was added at 0° C. The reaction was stirred for 3 hours at which point full conversion was observed. The reaction was quenched with sat. sodium bicarbonate solution, diluted with DCM, and passed through a phase separator. The organics were dried via rotovap and purification by silica gel chromatography (Gradient: 0-70% EtOAc in heptane) yielded the product 1-((2S)-1-(2,4-dimethoxybenzyl)-2-(1-methyl-1H-1,2,3-triazol-4-yl)-2′-(trifluoromethyl)-4′,5′-dihydrospiro[piperidine-4,7′-thieno[2,3-c]pyran]-3′-yl)ethan-1-ol C63 (290 mg, 74%). ¹H NMR (300 MHz, Chloroform-d) δ 7.56 (s, 1H), 7.24 (s, 1H), 6.56-6.36 (m, 2H), 5.32 (s, 2H), 5.25 (s, 1H), 4.04 (d, J=31.0 Hz, 6H), 3.79 (d, J=15.8 Hz, 6H), 3.67 (d, J=13.8 Hz, 1H), 3.22 (d, J=13.8 Hz, 1H), 3.03 (d, J=5.9 Hz, OH), 2.88-2.71 (m, 2H), 2.57 (d, J=11.9 Hz, 1H), 2.36 (d, J=13.8 Hz, 1H), 2.15-1.85 (m, 4H), 1.51 (dd, J=6.7, 2.7 Hz, 3H). LCMS m/z 553.05 [M+H]⁺.

### Step 4. Synthesis of 1-((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)-]113-ethan-1-ol (52)[DIAST-1] and (53)[DIAST-2]

1-((2S)-1-(2,4-dimethoxybenzyl)-2-(1-methyl-1H-1,2,3-triazol-4-yl)-2′-(trifluoromethyl)-4′,5′-dihydrospiro[piperidine-4,7′-thieno[2,3-c]pyran]-3′-yl)ethan-1-ol C63 (278 mg, 0.5031 mmol) in water (470 μL, 26.09 mmol) and TFA (1.36 mL, 17.65 mmol) was heated to 90° C. for 3 hours. The reaction mixture was cooled to room temperature, diluted with DCM, and then poured slowly into aq. 6 M NaOH. The organic layer was separated through a phase separator and concentrated. Purification by silica gel chromatography (Gradient: 0-20% MeOH in DCM) yielded a 1:1 diastereomeric mixture. This material was separated into constituent diastereomers by chiral SFC separation. Column: Daicel Chiralpak® AD-H, 10×250 mm; Mobile Phase: 20% IPA with 5 mM Ammonia, 80% carbon dioxide. 1-((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)-113-ethan-1-ol 52 [DIAST-1] (20.3 mg, 20%). LCMS m/z 403.09 [M+H]⁺. 1-((2S)-2-(1-methyl-1H-1,2,3-triazol-4-yl)-2′-(trifluoromethyl)-4′,5′-dihydrospiro[piperidine-4,7′-thieno[2,3-c]pyran]-3′-yl)-113-ethan-1-ol 53 [DIAST-2] (5.6 mg, 5%). LCMS m/z 403.09 [M+H]⁺.

### Preparation S38

### (2'S,7S)-2-chloro-2′-ethynyl-spiro[4,5-dihydrothieno[2,3-c]pyran-7,4′-piperidine](S38)

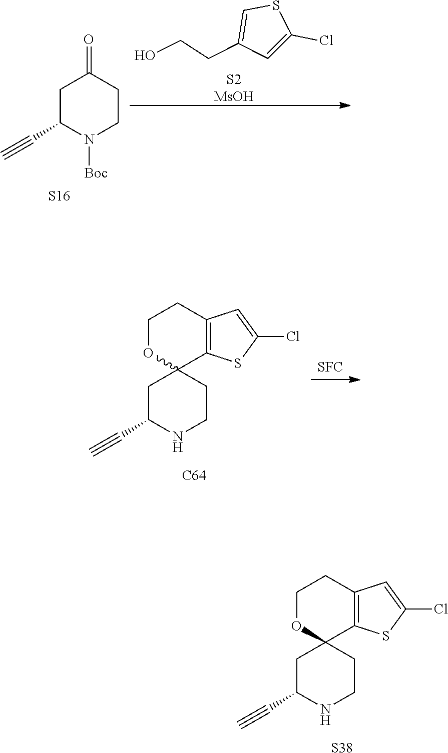

### Step 1. Synthesis of (2S)-2′-chloro-2-ethynyl-4′,5′-dihydrospiro[piperidine-4,7′-thieno[2,3-c]pyran] (C64)

To a mixture of tert-butyl (2S)-2-ethynyl-4-oxo-piperidine-1-carboxylate S16 (3100 mg, 13.88 mmol) in DCM (57 mL) was added 2-(5-chloro-3-thienyl)ethanol S2 (2 mL) followed by methanesulfonic acid (3.5 mL, 53.94 mmol). After stirring for 40 min, the mixture was washed with NaOH (9.3 mL of 6 M, 55.80 mmol) and diluted with 20 mL water and the organic layer was removed. The aqueous layer was extracted again with DCM (20 mL). The combined organic layers were washed with brine, dried with sodium sulfate and concentrated. Purification by silica gel chromatography (Gradient: 0-10% MeOH in DCM) afforded (2S)-2′-chloro-2-ethynyl-4′,5′-dihydrospiro[piperidine-4,7′-thieno[2,3-c]pyran] C64 (2.5 g, 56%) as a mixture of diastereomers. LCMS m/z 268.13 [M+H]⁺.

### Step 2. Synthesis of (2'S, 7S)-2-chloro-2′-ethynyl-spiro[4,5-dihydrothieno[2,3-c]pyran-7,4′-piperidine](S38)

(2S)-2′-chloro-2-ethynyl-4′,5′-dihydrospiro[piperidine-4,7′-thieno[2,3-c]pyran] C64 (2.5 g) was separated into constituent diastereomers by chiral SFC separation. Column: Phenomenex Lux® Cellulose-2, 20×250 mm; Mobile Phase: 20% MeOH with 5 mM Ammonia, 80% carbon dioxide. Peak A afforded (2'S,7S)-2-chloro-2′-ethynyl-spiro[4,5-dihydrothieno[2,3-c]pyran-7,4′-piperidine]S38 (350 mg, 9%) as a brown oil. ¹H NMR (300 MHz, Methanol-d₄) δ 6.68 (s, 1H), 3.91 (td, J=5.6, 1.5 Hz, 2H), 3.80 (dt, J=11.7, 2.6 Hz, 1H), 2.99 (td, J=12.7, 2.9 Hz, 1H), 2.84 (ddd, J=12.8, 4.8, 2.1 Hz, 1H), 2.70 (d, J=2.3 Hz, 1H), 2.62-2.56 (m, 2H), 2.22 (dt, J=13.8, 2.8 Hz, 1H), 2.01-1.92 (m, 1H), 1.83-1.73 (m, 1H), 1.74-1.63 (m, 1H). LCMS m/z 268.04 [M+H]⁺. Note: the stereochemistry of this intermediate was confirmed by using it to synthesize Compound 2 and finding the data was convergent.

### Compound 54

### 3-(4-((2S,4S)-2′-chloro-4′,5′-dihydrospiro[piperidine-4,7′-thieno[2,3-c]pyran]-2-yl)-1H-1,2,3-triazol-1-yl)benzamide (54)

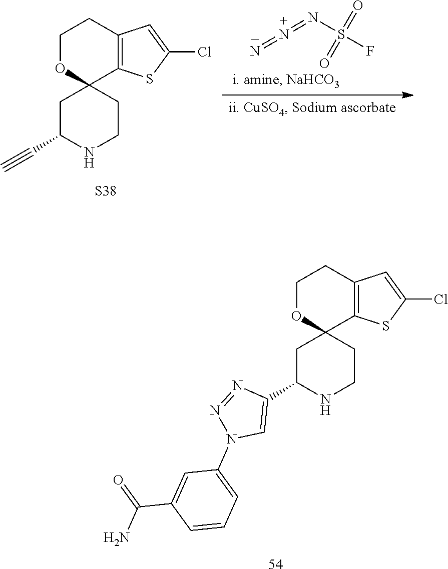

To a mixture of 3-aminobenzamide (33.86 mg, 0.2487 mmol) in DMSO (0.5 mL) was added aqueous sodium bicarbonate (174.1 μL of 12% w/v, 0.2487 mmol) followed by a solution of N-diazosulfamoyl fluoride (31.11 mg, 0.2487 mmol) in MTBE (0.54 mL) (prepared according to literature precedent in Meng, G., Guo, T., Ma, T. et al. Modular click chemistry libraries for functional screens using a diazotizing reagent. Nature 574, 86-89 (2019)). The mixture was stirred vigorously for 5 min. At this time (2′S,7S)-2-chloro-2′-ethynyl-spiro[4,5-dihydrothieno[2,3-c]pyran-7,4′-piperidine]S38 (20 mg, 0.07469 mmol) in DMSO (0.4 mL) was added followed by aqueous CuSO4 (59.61 μL of 1% w/v, 0.003735 mmol) and sodium ascorbate (2.631 mg, 0.01494 mmol). The mixture was heated to 50° C. open to air and stirred overnight. The mixtures had evaporated the residual MTBE and were directly purified by reversed-phase HPLC (Method: C18 Waters Sunfire column (30×150 mm, 5 micron). Gradient: MeCN in H₂O with 5 mM HCl) to yield 3-(4-((2S,4S)-2′-chloro-4′,5′-dihydrospiro[piperidine-4,7′-thieno[2,3-c]pyran]-2-yl)-1H-1,2,3-triazol-1-yl)benzamide 54 (18.3 mg, 52%) as the hydrochloride salt. ¹H NMR (300 MHz, Methanol-d₄) δ 8.74 (s, 1H), 8.37 (t, J=1.9 Hz, 1H), 8.11-7.97 (m, 2H), 7.71 (t, J=8.0 Hz, 1H), 6.78 (s, 1H), 4.99 (dd, J=12.6, 3.1 Hz, 1H), 4.06 (td, J=5.6, 2.4 Hz, 2H), 3.68-3.55 (m, 1H), 3.51-3.40 (m, 1H), 2.70 (t, J=5.4 Hz, 2H), 2.66 (s, 3H), 2.50-2.33 (m, 2H), 2.13 (td, J=14.6, 14.2, 4.8 Hz, 1H). LCMS m/z 430.19 [M+H]⁺.

### Compound 55

### (2S,4S)-2′-chloro-2-(1-phenyl-1H-1,2,3-triazol-4-yl)-4′,5′-dihydrospiro[piperidine-4,7′-thieno[2,3-c]pyran] (55)

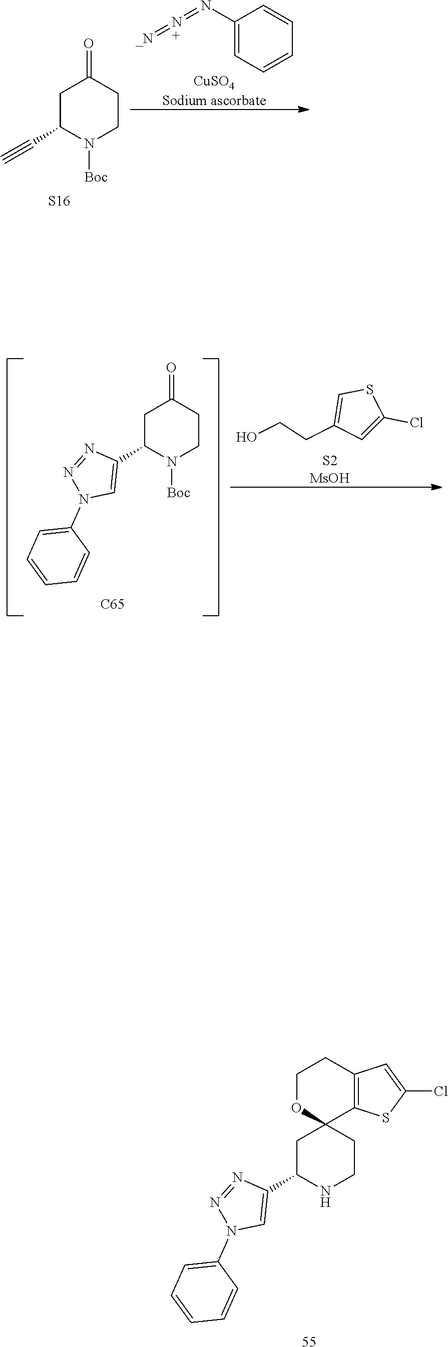

### Preparation of (2S,4S)-2′-chloro-2-(1-phenyl-1H-1,2,3-triazol-4-yl)-4′,5′-dihydrospiro[piperidine-4,7′-thieno[2,3-c]pyran] (55)

To azidobenzene (16 mg, 0.1343 mmol) in methanol (800.0 μL) was added aqueous copper (II) sulfate (100 μL of 1% w/v, 0.006265 mmol), and to the pale blue solution was added tert-butyl (2S)-2-ethynyl-4-oxo-piperidine-1-carboxylate S16 (20 mg, 0.08958 mmol) followed by sodium ascorbate (2 mg, 0.01136 mmol). After stirring overnight, the mixture was concentrated and the residue diluted in DCM and sat. aq. sodium bicarbonate. The organics were collected via a phase separator affording a crude solution of C65. To this was added MsOH (25 μL, 0.3853 mmol) followed by 2-(5-chloro-3-thienyl)ethanol S2 (25 mg, 0.1537 mmol), and the mixture was stirred overnight. At this time, the mixture was pH adjusted with sat. aq. sodium bicarbonate and the organics collected via a phase separator and blown down with nitrogen. Purification by reversed-phase HPLC (Method: C18 Waters Sunfire column (30×150 mm, 5 micron). Gradient: MeCN in H₂O with 0.1% trifluoroacetic acid) yielded (2S,4S)-2′-chloro-2-(1-phenyl-1H-1,2,3-triazol-4-yl)-4′,5′-dihydrospiro[piperidine-4,7′-thieno[2,3-c]pyran] 55 (24.9 mg, 55%) as a trifluoroacetate salt. ¹H NMR (300 MHz, Methanol-d₄) δ 8.67 (d, J=0.5 Hz, 1H), 7.90-7.76 (m, 2H), 7.65-7.55 (m, 2H), 7.55-7.42 (m, 1H), 6.76 (s, 1H), 4.97 (dd, J=12.4, 3.2 Hz, 1H), 4.05 (td, J=5.6, 2.2 Hz, 2H), 3.61 (td, J=13.1, 3.2 Hz, 1H), 3.46 (ddd, J=12.9, 4.8, 2.1 Hz, 1H), 2.73-2.61 (m, 3H), 2.45 (dd, J=14.7, 12.5 Hz, 1H), 2.41-2.32 (m, 1H), 2.14 (ddd, J=14.9, 13.3, 4.8 Hz, 1H). LCMS m/z 387.25 [M+H]⁺.

### Compounds 56-77

Compounds 56-77 (see Table 5) were prepared by methods similar to compounds 54 or 55 with modifications obvious to someone skilled in the art. Amines or azides were obtained from commercial sources.

TABLE 5
Preparation method, structure and physicochemical data for compounds 56-77.

| | | Azide or | | ¹H NMR; LCMS |
|---|---|---|---|---|
| Compound | Product | Amine | Method | m/z [M + H]⁺ |
| 56 | 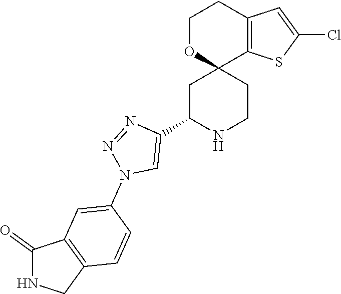 [SMILES: O=C1NCc2ccc(-n3cc([C@@H]4C[C@]5(CCN4)OCCc4cc(Cl)sc45)nn3)cc21] | 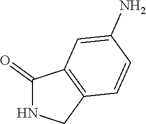 [SMILES: Nc1ccc2c(c1)C(=O)NC2] | Compound 54¹ | LCMS m/z 442.11 [M + H]⁺ |
| 57 | 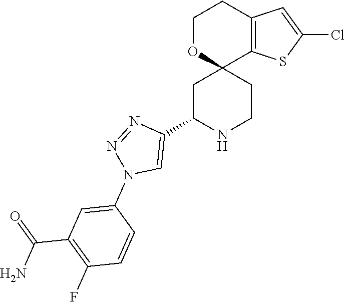 [SMILES: NC(=O)c1cc(-n2cc([C@@H]3C[C@]4(CCN3)OCCc3cc(Cl)sc34)nn2)ccc1F] | 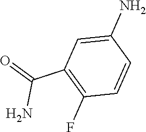 [SMILES: NC(=O)c1cc(N)ccc1F] | Compound 54¹ | LCMS m/z 448.12 [M + H]⁺ |
| 58 | 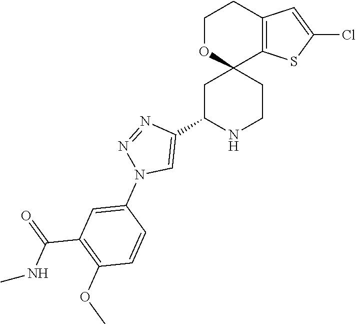 [SMILES: CNC(=O)c1cc(-n2cc([C@@H]3C[C@]4(CCN3)OCCc3cc(Cl)sc34)nn2)ccc1OC] | 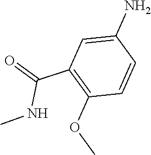 | Compound 54¹ | LCMS m/z 474.12 [M + H]⁺ |
| 59 | 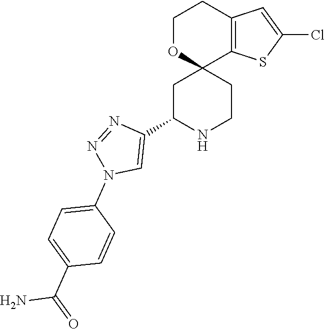 [SMILES: NC(=O)c1ccc(-n2cc([C@@H]3C[C@]4(CCN3)OCCc3cc(Cl)sc34)nn2)cc1] | 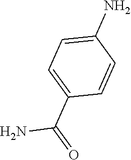 | Compound 54² | ¹H NMR (400 MHz, DMSO-d₆) δ 9.65 (s, 2H), 9.12 (s, 1H), 8.17 (s, 1H), 8.12 (d, J = 8.7 Hz, 2H), 8.00 (d, J = 8.5 Hz, 2H), 7.56 (s, 1H), 6.96 (s, 1H), 4.73 (d, J = 12.1 Hz, 1H), 3.98 (p, J = 5.9, 5.5 Hz, 2H), 3.32 (s, 2H), 2.63 (t, J = 5.4 Hz, 2H), 2.61-2.34 (m, 2H), 2.20 (q, J = 11.7, 8.7 Hz, 2H). LCMS m/z 430.19 [M + H]⁺ |
| 60 | 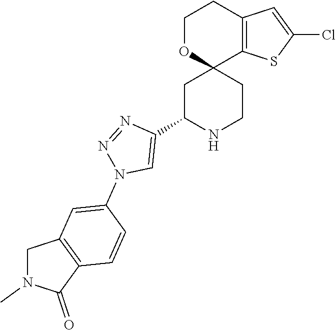 [SMILES: CN1Cc2cc(-n3cc([C@@H]4C[C@]5(CCN4)OCCc4cc(Cl)sc45)nn3)ccc2C1=O] | 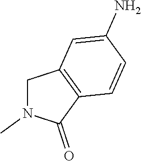 | Compound 54¹ | LCMS m/z 456.11 [M + H]⁺ |
| 61 | 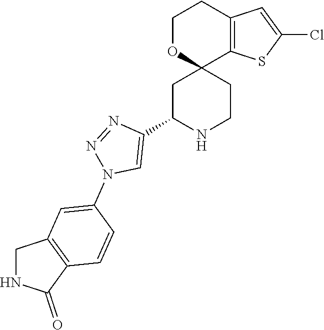 [SMILES: O=C1NCc2cc(-n3cc([C@@H]4C[C@]5(CCN4)OCCc4cc(Cl)sc45)nn3)ccc21] | 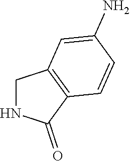 [SMILES: Nc1ccc2c(c1)CNC2=O] | Compound 54¹ | LCMS m/z 442.11 [M + H]⁺ |
| 62 | 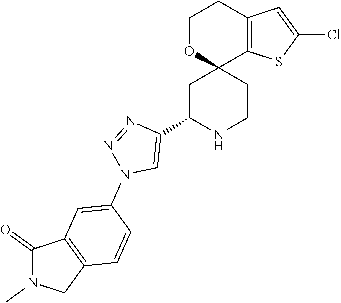 [SMILES: CN1Cc2ccc(-n3cc([C@@H]4C[C@]5(CCN4)OCCc4cc(Cl)sc45)nn3)cc2C1=O] | 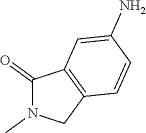 | Compound 54¹ | LCMS m/z 456.11 [M + H]⁺ |
| 63 | 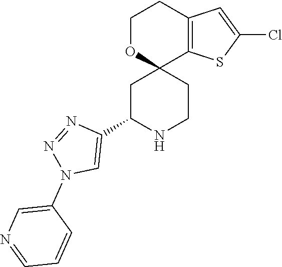 [SMILES: Clc1cc2c(s1)[C@@]1(CCN[C@H](c3cn(-c4cccnc4)nn3)C1)OCC2] | 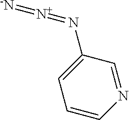 | Compound 55 | ¹H NMR (400 MHz, Chloroform-d) δ 8.92 (dd, J = 5.2, 2.5 Hz, 1H), 8.63 (dd, J = 4.8, 1.5 Hz, 1H), 8.05 (tdd, J = 8.2, 2.7, 1.5 Hz, 1H), 7.90 (s, 1H), 7.48- 7.33 (m, 1H), 6.56 (d, J = 28.4 Hz, 1H), 4.41 (td, J = 11.7, 10.7, 3.3 Hz, 1H), 3.97-3.68 (m, 2H), 3.30-3.12 (m, 1H), 3.12-2.90 (m, 1H), 2.64-2.46 (m, 2H), |
| | | | | 2.46-2.27 (m, 1H), |
| | | | | 2.25-1.98 (m, 1H), |
| | | | | 1.85-1.69 (m, 2H). |
| | | | | LCMS m/z 388.15 |
| | | | | [M + H]⁺ |
| 64 | 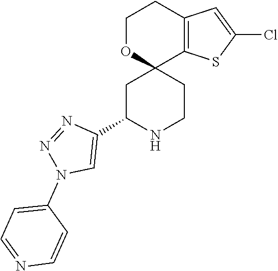 [SMILES: Clc1cc2c(s1)[C@@]1(CCN[C@H](c3cn(-c4ccncc4)nn3)C1)OCC2] | 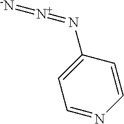 | Compound 55³ | ¹H NMR (300 MHz, Methanol-d₄) δ 9.05 (d, J = 0.6 Hz, 1H), 8.96-8.87 (m, 2H), 8.35-8.26 (m, 2H), 6.77 (s, 1H), 5.02 (dd, J = 12.4, 3.2 Hz, 1H), 4.06 (td, J = 5.6, 2.0 Hz, 2H), 3.62 (td, J = 13.1, 3.2 Hz, 1H), 3.48 (ddd, J = 13.0, 4.9, 2.0 Hz, 1H), 2.75- 2.61 (m, 3H), 2.46 (dd, J = 14.6, 12.5 Hz, 1H), 2.42-2.33 (m, 1H), 2.15 (ddd, |
| | | | | J = 14.8, 13.2, 4.8 Hz, |
| | | | | 1H). LCMS m/z |
| | | | | 388.24 [M + H]⁺ |
| 65 | 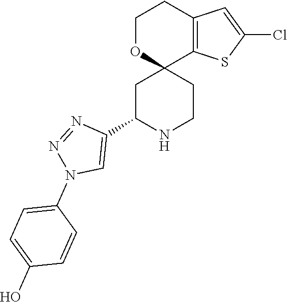 [SMILES: Oc1ccc(-n2cc([C@@H]3C[C@]4(CCN3)OCCc3cc(Cl)sc34)nn2)cc1] | 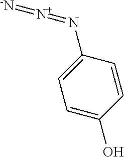 | Compound 55^{2,4} | ¹H NMR (300 MHz, Methanol-d₄) δ 8.49 (s, 1H), 7.66-7.56 (m, 2H), 7.01-6.91 (m, 2H), 6.78 (s, 1H), 4.94 (dd, J = 12.4, 3.2 Hz, 1H), 4.05 (td, J = 5.6, 2.2 Hz, 2H), 3.70-3.52 (m, 1H), 3.52-3.40 (m, 1H), 2.74-2.62 (m, 3H), 2.40 (dd, J = 14.8, 12.5 Hz, 2H), 2.11 (td, J = 14.0, 4.7 Hz, 1H). LCMS m/z 403.19 [M + H]⁺ |
| 66 | 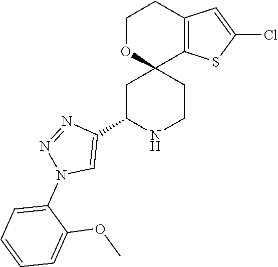 [SMILES: COc1ccccc1-n1cc([C@@H]2C[C@]3(CCN2)OCCc2cc(Cl)sc23)nn1] | 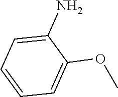 | Compound 54² | ¹H NMR (300 MHz, Methanol-d₄) δ 8.53 (s, 1H), 7.66 (dd, J = 7.9, 1.7 Hz, 1H), 7.54 (ddd, J = 8.5, 7.5, 1.7 Hz, 1H), 7.29 (dd, J = 8.4, 1.2 Hz, 1H), 7.14 (td, J = 7.7, 1.2 Hz, 1H), 6.77 (s, 1H), 4.97 (dd, J = 12.4, 3.2 Hz, 1H), 4.05 (td, J = 5.6, 2.3 Hz, 2H), 3.91 (s, 3H), 3.60 (td, J = 13.1, 3.3 Hz, 1H), 3.45 (ddd, J = 12.9, 4.9, 2.1 Hz, |
| | | | | 1H), 2.73-2.58 (m, |
| | | | | 3H), 2.52-2.43 (m, |
| | | | | 1H), 2.43-2.33 (m, |
| | | | | 1H), 2.16 (ddd, J = |
| | | | | 14.8, 13.2, 4.8 Hz, |
| | | | | 1H). LCMS m/z |
| | | | | 417.18 [M + H]⁺ |
| 67 | 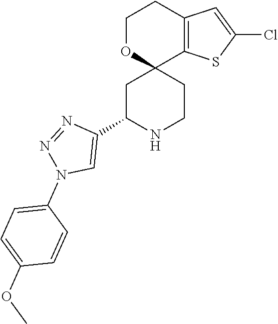 [SMILES: COc1ccc(-n2cc([C@@H]3C[C@]4(CCN3)OCCc3cc(Cl)sc34)nn2)cc1] | 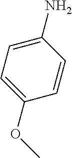 | Compound 54² | ¹H NMR (400 MHz, DMSO-d₆) δ 9.65 (s, 2H), 8.93 (s, 1H), 7.82-7.75 (m, 2H), 7.20-7.14 (m, 2H), 6.96 (s, 1H), 4.71 (d, J = 12.0 Hz, 1H), 3.97 (h, J = 6.4 Hz, 2H), 3.84 (s, 3H), 3.31 (ddd, J = 7.9, 3.8, 2.1 Hz, 2H), 2.63 (t, J = 5.4 Hz, 2H), 2.59-2.30 (m, 2H), 2.20 (q, J = 11.6, 8.5 Hz, 2H). LCMS m/z 417.18 [M + H]⁺ |
| 68 | 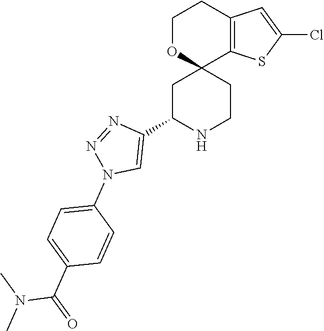 [SMILES: CN(C)C(=O)c1ccc(-n2cc([C@@H]3C[C@]4(CCN3)OCCc3cc(Cl)sc34)nn2)cc1] | 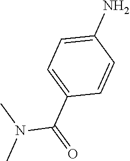 | Compound 54¹ | LCMS m/z 458.14 [M + H]⁺ |
| 69 | 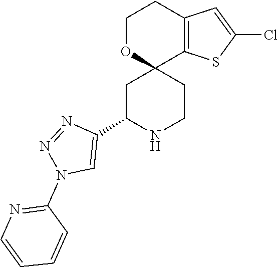 [SMILES: Clc1cc2c(s1)[C@@]1(CCN[C@H](c3cn(-c4ccccn4)nn3)C1)OCC2] | 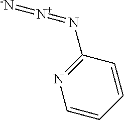 | Compound 55³ | ¹H NMR (300 MHz, Methanol-d₄) δ 8.92 (s, 1H), 8.62-8.52 (m, 1H), 8.18 (ddt, J = 8.2, 3.4, 1.0 Hz, 1H), 8.08 (ddd, J = 8.2, 7.4, 1.9 Hz, 1H), 7.51 (ddt, J = 7.4, 4.9, 1.2 Hz, 1H), 6.77 (d, J = 2.6 Hz, 1H), 5.14-4.95 (m, 1H), 4.13-3.54 (m, 3H), 3.46 (ddt, J = 10.9, 6.1, 3.2 Hz, 1H), 2.85-2.61 (m, 3H), 2.57-2.30 (m, 2H), 2.29-2.05 (m, |
| | | | | 1H). LCMS m/z |
| | | | | 388.24 [M + H]⁺ |
| 70 | 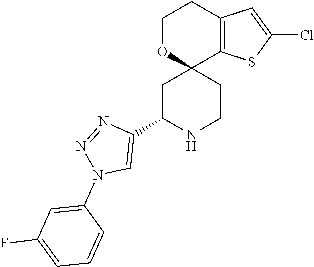 [SMILES: Fc1cccc(-n2cc([C@@H]3C[C@]4(CCN3)OCCc3cc(Cl)sc34)nn2)c1] | 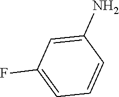 | Compound 54² | ¹H NMR (400 MHz, DMSO-d₆) δ 9.65 (s, 2H), 9.10 (s, 1H), 7.87-7.76 (m, 2H), 7.69 (td, J = 8.2, 6.2 Hz, 1H), 7.41 (td, J = 8.5, 2.5 Hz, 1H), 6.96 (s, 1H), 4.72 (d, J = 11.6 Hz, 1H), 3.97 (q, J = 5.4 Hz, 2H), 3.34-3.28 (m, 2H), 2.63 (t, J = 5.4 Hz, 2H), 2.59-2.34 (m, 2H), 2.20 (q, J = 11.7, 8.4 Hz, 2H). LCMS m/z 405.17 [M + H]⁺ |
| 71 | 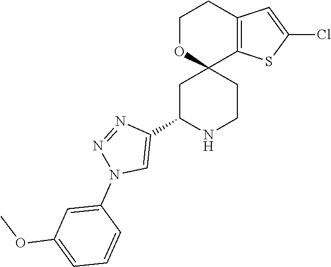 [SMILES: COc1cccc(-n2cc([C@@H]3C[C@]4(CCN3)OCCc3cc(Cl)sc34)nn2)c1] | 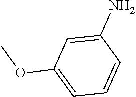 | Compound 54² | ¹H NMR (400 MHz, DMSO-d₆) δ 9.65 (s, 2H), 9.05 (s, 1H), 7.54 (t, J = 8.1 Hz, 1H), 7.49-7.42 (m, 2H), 7.13-7.08 (m, 1H), 6.96 (s, 1H), 4.72 (d, J = 11.5 Hz, 1H), 3.97 (h, J = 6.4 Hz, 2H), 3.86 (s, 3H), 3.34-3.27 (m, 2H), 2.63 (t, J = 5.4 Hz, 2H), 2.60-2.30 (m, 2H), 2.29-2.10 (m, 2H). LCMS m/z 417.18 [M + H]⁺ |
| 72 | 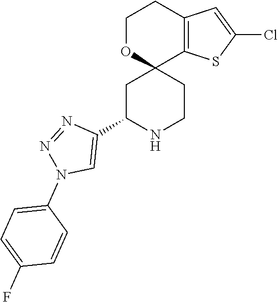 [SMILES: Fc1ccc(-n2cc([C@@H]3C[C@]4(CCN3)OCCc3cc(Cl)sc34)nn2)cc1] | 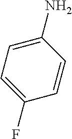 | Compound 54² | ¹H NMR (400 MHz, DMSO-d₆) δ 9.64 (s, 2H), 9.02 (s, 1H), 7.97-7.89 (m, 2H), 7.50 (t, J = 8.8 Hz, 2H), 6.96 (s, 1H), 4.72 (d, J = 12.1 Hz, 1H), 3.97 (h, J = 6.3 Hz, 2H), 3.34-3.25 (m, 2H), 2.66-2.59 (m, 2H), 2.59-2.34 (m, 2H), 2.20 (q, J = 11.7, 8.5 Hz, 2H). LCMS m/z 405.17 [M + H]⁺ |
| 73 | 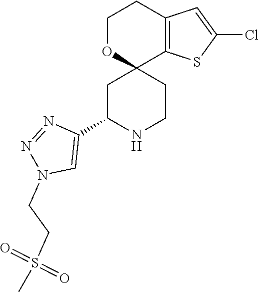 [SMILES: CS(=O)(=O)CCn1cc([C@@H]2C[C@]3(CCN2)OCCc2cc(Cl)sc23)nn1] | 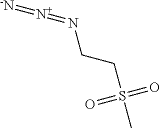 | Compound 55 | ¹H NMR (400 MHz, Chloroform-d) δ 7.62 (d, J = 0.6 Hz, 1H), 6.61 (s, 1H), 4.85 (dd, J = 6.9, 6.0 Hz, 2H), 4.39 (dd, J = 11.8, 2.6 Hz, 1H), 3.98 (td, J = 5.5, 1.5 Hz, 2H), 3.74-3.68 (m, 2H), 3.27 (td, J = 12.4, 2.7 Hz, 1H), 3.06 (ddd, J = 12.2, 4.7, 2.1 Hz, 1H), 2.73 (d, J = 0.7 Hz, 3H), 2.66-2.62 (m, 2H), 2.38 (dt, J = 13.5, 2.6 Hz, 1H), 2.09 (dd, J = 13.9, |
| | | | | 2.6 Hz, 1H), 1.89- |
| | | | | 1.77 (m, 2H). LCMS |
| | | | | m/z 417.22 [M + H]⁺ |
| 74 | 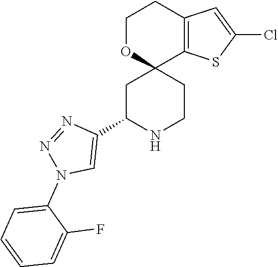 [SMILES: Fc1ccccc1-n1cc([C@@H]2C[C@]3(CCN2)OCCc2cc(Cl)sc23)nn1] | 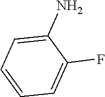 | Compound 54² | ¹H NMR (300 MHz, DMSO-d₆) δ 9.85 (s, 2H), 8.95 (d, J = 2.1 Hz, 1H), 7.93 (td, J = 7.9, 1.6 Hz, 1H), 7.81-7.63 (m, 2H), 7.55 (ddd, J = 8.2, 6.4, 2.1 Hz, 1H), 7.03 (s, 1H), 4.85 (s, 1H), 4.16-4.00 (m, 2H), 3.96 (s, 1H), 3.39 (s, 2H), 2.69 (q, J = 7.8, 6.6 Hz, 2H), 2.61 (d, J = 3.1 Hz, 1H), 2.29 (d, J = 6.5 Hz, 2H). LCMS m/z 405.17 [M + H]⁺ |
| 75 | 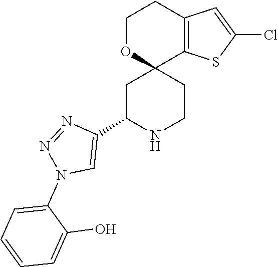 [SMILES: Oc1ccccc1-n1cc([C@@H]2C[C@]3(CCN2)OCCc2cc(Cl)sc23)nn1] | 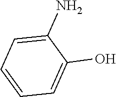 | Compound 54² | ¹H NMR (300 MHz, DMSO-d₆) δ 10.80 (s, 1H), 9.58 (s, 2H), 8.76 (s, 1H), 7.62 (dd, J = 8.0, 1.6 Hz, 1H), 7.37 (ddd, J = 8.8, 7.4, 1.7 Hz, 1H), 7.19 (dd, J = 8.3, 1.3 Hz, 1H), 7.02 (qd, J = 7.8, 7.0, 1.3 Hz, 1H), 6.96 (s, 1H), 4.78 (t, J = 11.2 Hz, 1H), 3.98 (q, J = 5.4 Hz, 2H), 3.32 (s, 2H), 2.70-2.59 (m, 2H), 2.54 (d, J = 2.8 Hz, 1H), 2.41 (dd, |
| | | | | J = 14.4, 12.3 Hz, |
| | | | | 1H), 2.27-2.07 (m, |
| | | | | 2H). LCMS m/z |
| | | | | 403.19 [M + H]⁺ |
| 76 | 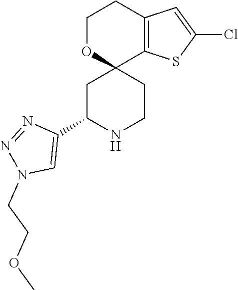 [SMILES: COCCn1cc([C@@H]2C[C@]3(CCN2)OCCc2cc(Cl)sc23)nn1] | 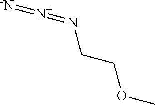 | Compound 55³ | ¹H NMR (400 MHz, DMSO-d₆) δ 9.38 (s, 1H), 9.11 (s, 1H), 8.24 (s, 1H), 6.95 (d, J = 1.1 Hz, 1H), 4.68 (d, J = 12.1 Hz, 1H), 4.58 (t, J = 5.0 Hz, 2H), 3.97 (hept, J = 5.9 Hz, 2H), 3.73 (t, J = 5.1 Hz, 2H), 3.30 (d, J = 7.7 Hz, 2H), 3.24 (d, J = 0.9 Hz, 3H), 2.62 (t, J = 5.5 Hz, 2H), 2.47 (s, 1H), 2.32- 2.17 (m, 2H), 2.03 (dt, J = 15.7, 8.9 Hz, 1H). LCMS m/z 369.32 [M + H]⁺ |
| 77 | 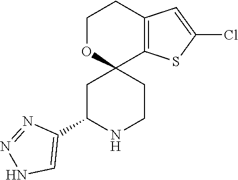 [SMILES: Clc1cc2c(s1)[C@@]1(CCN[C@H](c3c[nH]nn3)C1)OCC2] | 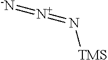 | Compound 55³ | ¹H NMR (400 MHz, Chloroform-d) δ 7.75 (s, 1H), 6.65 (s, 1H), 4.92 (d, J = 12.5 Hz, 1H), 3.99 (s, 2H), 3.63 (s, 1H), 3.51 (d, J = 11.6 Hz, 1H), 2.68 (t, J = 5.5 Hz, 2H), 2.56 (s, 1H), 2.47 (s, 1H), 2.34 (s, 2H). LCMS m/z 311.1 [M + H]⁺ |

Footnotes:
¹The product was isolated as the formate salt.
²The product was isolated as the hydrochloride salt.
³The product was isolated as the trifluoroacetate salt.
⁴The click reaction was performed on S38 to afford the product.

### Compound 78

### 2-ethyl-2′-(4-fluorophenyl)spiro[6,7-dihydrothieno[3,2-c]pyran-4,4′-piperidine] (78)

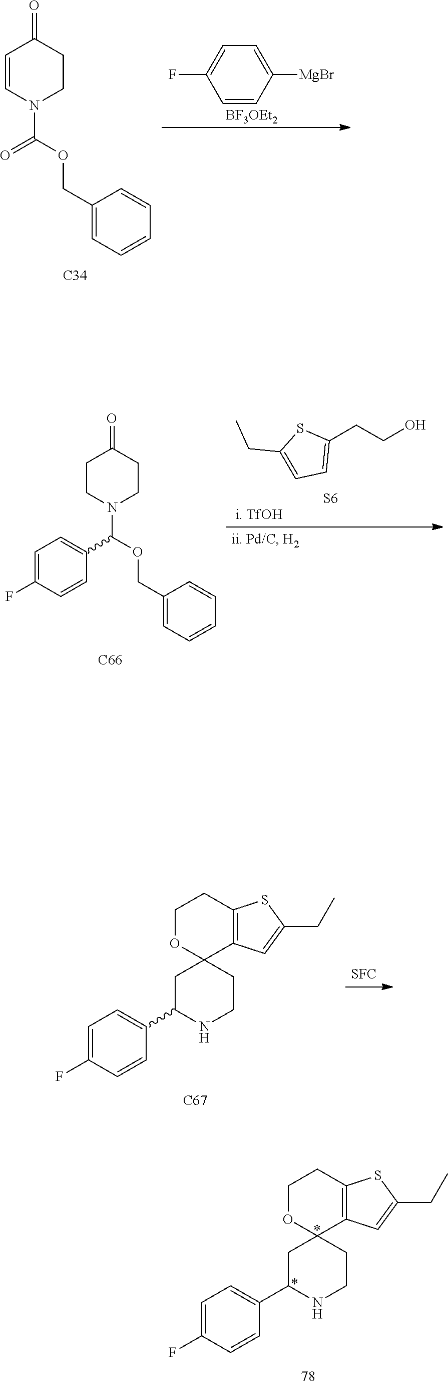

### Step 1. Synthesis of benzyl 2-(4-fluorophenyl)-4-oxo-piperidine-1-carboxylate (C₆₅)

A solution of copper(I) bromide dimethyl sulfide complex (1.5 g, 7.296 mmol) in THF (25 mL) was cooled to −78° C. 4-fluorophenylmagnesium bromide (7.3 mL of 1 M in THF, 7.300 mmol) was added slowly via addition funnel. After stirring at −78° C. for 1 hour, diethyloxonio(trifluoro)boranuide (896 μL, 7.260 mmol) was added and stirred for 5 minutes. To the newly formed complex was then added a solution of benzyl 4-oxo-2,3-dihydropyridine-1-carboxylate C34 (999.0 mg, 4.32 mmol) in THF (15 mL) slowly over one hour. After stirring 2 hours at −78° C., 16 mL of aq. 20% NH₄Cl/conc. NH₄OH (1:1) were added, and the mixture was warmed to room temperature. The solution was extracted with EtOAc (3×100 mL) and the combined organic layers were washed with brine, dried over MgSO₄, and concentrated. Purification by silica gel chromatography yielded the product benzyl 2-(4-fluorophenyl)-4-oxo-piperidine-1-carboxylate C66 (883 mg, 33%). LCMS m/z 327.93 [M+H]⁺.

### Step 2. Synthesis of 2-ethyl-2′-(4-fluorophenyl)spiro[6,7-dihydrothieno[3,2-c]pyran-4,4′-piperidine] (C67)

A solution of benzyl 2-(4-fluorophenyl)-4-oxo-piperidine-1-carboxylate C66 (115 mg, 0.3513 mmol) and 2-(5-ethyl-2-thienyl)ethanol S6 (55 mg, 0.3520 mmol) in dioxane (2 mL) was cooled to 0° C. and treated with trifluoromethanesulfonic acid (95 μL, 1.074 mmol) dropwise. The mixture was stirred at 0° C. for 30 min and warmed to room temperature. After another 30 min sat. sodium bicarbonate solution was added and the mixture was extracted with DCM (3×3 mL). The combined organics were dried over sodium sulfate and concentrated in vacuo. Purification by silica gel chromatography (Gradient: 0-30% EtOAc in Hexanes) afforded the CBz protected intermediate which was immediately treated with Pd/C (38 mg of 10 wt %, 0.03571 mmol) and brought up in MeOH (10 mL). The reaction was put purged and evacuated (3×) and stirred under a hydrogen balloon atmosphere. After one hour the mixture was filtered through a pad of celite and the filtrate concentrated. Purification by silica gel chromatography (Gradient: 0-10% 0.7 M ammonia in MeOH in DCM) provided 2-ethyl-2′-(4-fluorophenyl)spiro[6,7-dihydrothieno[3,2-c]pyran-4,4′-piperidine] C67 (32 mg, 26%). LCMS m/z 332.03 [M+H]⁺.

### Step 3. Synthesis of 2-ethyl-2′-(4-fluorophenyl)spiro[6,7-dihydrothieno[3,2-c]pyran-4,4′-piperidine] (78)

The racemic compound 2-ethyl-2′-(4-fluorophenyl)spiro[6,7-dihydrothieno[3,2-c]pyran-4,4′-piperidine] C67 (26 mg, 0.07292 mmol) was separated into four constituent stereoisomers by chiral SFC separation. Column: Daicel Chiralpak® AD-H, 10×250 mm; Mobile Phase: 15% EtOH (5 mM ammonia), 85% carbon dioxide. Peak A afforded 2-ethyl-2′-(4-fluorophenyl)spiro[6,7-dihydrothieno[3,2-c]pyran-4,4′-piperidine]78 (3.2 mg, 42%). ¹H NMR (300 MHz, Chloroform-d) δ 7.53-7.31 (m, 2H), 7.10-6.90 (m, 2H), 6.50 (d, J=1.1 Hz, 1H), 4.09 (dd, J=11.5, 2.6 Hz, 1H), 4.05-3.91 (m, 2H), 3.37-3.17 (m, 1H), 3.14-2.94 (m, 1H), 2.87-2.71 (m, 4H), 2.06-1.87 (m, 3H), 1.80 (dd, J=13.6, 11.6 Hz, 1H), 1.31-1.25 (m, 4H). LCMS m/z 331.53 [M+H]⁺.

### Example 2. Assays for Detecting and Measuring APOL1 Inhibitor Properties of Compounds

### MultiTox-Fluor Multiplex Cytotoxicity Assay

The MultiTox-Fluor Multiplex Cytotoxicity Assay is a single-reagent-addition, homogeneous, fluorescence assay that measures the number of live and dead cells simultaneously in culture wells. The assay measures cell viability and cytotoxicity by detecting two distinct protease activities. The live-cell protease activity is restricted to intact viable cells and is measured using a fluorogenic, cell-permeant peptide glycyl-phenylalanylamino fluorocoumarin (GF-AFC) substrate. The substrate enters intact cells, where it is cleaved to generate a fluorescent signal proportional to the number of living cells. This live-cell protease activity marker becomes inactive upon loss of membrane integrity and leakage into the surrounding culture medium. A second, cell-impermeant, fluorogenic peptide substrate (bis-AAF-R110 Substrate) is used to measure dead-cell protease that has been released from cells that have lost membrane integrity. A ratio of dead to live cells is used to normalize data.

Briefly, the tet-inducible transgenic APOL1 T-REx-HEK293 cell lines were incubated with 50 ng/mL tet to induce APOL1 in the presence of 3-(2-(4-fluorophenyl)-1H-indol-3-yl)-N-((3S,4R)-4-hydroxy-2-oxopyrrolidin-3-yl)propenamide at 10.03, 3.24, 1.13, 0.356, 0.129, 0.042, 0.129, 0.0045, 0.0015, 0.0005 μM in duplicate for 24 hours in a humidified 37° C. incubator. The MultiTox reagent was added to each well and placed back in the incubator for an additional 30 minutes. The plate was read on the EnVision plate reader. A ratio of dead to live cells was used to normalize, and data was imported, analyzed, and fit using Genedata Screener (Basel, Switzerland) software. Data was normalized using percent of control, no tet (100% viability), and 50 ng/mL tet treated (0% viability),and fit using Smart Fit. The reagents, methods, and complete protocol for the MultiTox assay are described below.

TABLE 6
Reagents Used in the Multi-Tox Assay

| Reagent | Catalog Number | Vendor |
|---|---|---|
| 384 well, transparent, flat | 356663 | Corning (Corning, NY) |
| bottom tissue culture | | |
| treated, Poly-D lysine | | |
| coated | | |
| 384 well round bottom | 3656 | CoStar (Corning, NY) |
| polypropylene plates | | |
| Universal plate lids | 250002 | Thermo Fisher |
| | | (Waltham) |
| Axygen 30 μL tips for | VT-384-31UL-R-S | Corning (Corning, NY) |
| Bravo 384 well | | |
| MultiTox-Fluor Multiplex | G9202 | Promega (Madison, |
| Cytotoxicity Assay | | WI) |
| 225 cm² flask, angled neck, | 431082 | Corning (Corning, NY) |
| treated, vented cap | | |
| Dulbecco's Phosphate- | 14190-136 | Thermo Fisher |
| Buffered Saline (DPBS), | | (Waltham) |
| calcium and magnesium- | | |
| free | | |
| Dulbecco's Modified Eagle | 11960-077 | Thermo Fisher |
| Medium (DMEM), high | | (Waltham) |
| glucose, no glutamine, no | | |
| sodium pyruvate | | |
| Fetal Bovine Serum (FBS), | 631368 | Takara (Kusatsu, |
| tetracycline-free, US- | | Japan) |
| Sourced | | |
| L-Glutamine, 200 mM | 25030-081 | Thermo Fisher |
| | | (Waltham) |
| Penicillin-Streptomycin, | 15140-122 | Thermo Fisher |
| 10,000 Units/mL | | (Waltham) |
| Blasticidin S HCl, 10 | A11139-03 | Thermo Fisher |
| mg/mL | | (Waltham) |
| Tetracycline hydrochloride | T7660-5G | Sigma (St. Louis, MO) |
| Puromycin | A11138-03 | Thermo Fisher |
| dihydrochloride, 10 mg/mL | | (Waltham) |
| Trypsin-EDTA | 25300-054 | Thermo Fisher |
| | | (Waltham) |

TABLE 7
Equipment Used in the Multi-Tox Assay

| Instrument | Model | Supplier | Location |
|---|---|---|---|
| Bravo | 16050-101 | Agilent | Santa Clara, CA |
| | | Technologies | |
| Multidrop | N/A | Thermo | Waltham, MA |
| Combi | | Scientific | |
| EnVision | N/A | PerkinElmer | Waltham, MA |

### Multi-Tox Assay Protocol

Human embryonic kidney (HEK293) cell lines containing a tet-inducible expression system (T-REx™; Invitrogen, Carlsbad, CA) and Adeno-associated virus site 1 pAAVS1-Puro-APOL1 G0 or pAAVS1-Puro-APOL1 G1 or pAAVS1-Puro-APOL1 G2 Clones G0 DC2.13, G1 DC3.25, and G2 DC4.44 were grown in a T-225 flask at ˜90% confluency in cell growth media (DMEM, 10% Tet-free FBS, 2 mM L-glutamine, 100 Units/mL penicillin-streptomycin, 5 μg/mL blasticidin S HCl, 1 μg/mL puromycin dihydrochloride). Cells were washed with DPBS and then trypsinized to dissociate from the flask. Media was used to quench the trypsin, cells were then pelleted at 200 g and resuspended in fresh cell assay media (DMEM, 2% Tet-free FBS, 2 mM L-glutamine, 100 Units/mL penicillin-streptomycin). Cells were counted and diluted to 1.17×10⁶ cells/mL. 20 μL of cells (23,400/well) were dispensed in every well of a 384-well Poly-D-Lysine coated plate using the Multidrop dispenser. The plates were then incubated at room temperature for one hour.

Tetracycline is needed to induce APOL1 expression. 1 mg/mL tet stock in water was diluted to 250 ng/mL (5×) in cell assay media. 60 μL of cell assay media (no tet control) was dispensed in columns 1 and 24, and 60 μL of 5×tet in 384-PP-round bottom plate was dispensed in columns 2 to 23 with the Multidrop dispenser.

Assay ready plates from the Global Compound Archive were ordered using template 384_APOL1Cell_DR10n2_50 uM_v3. Compounds were dispensed at 200 nL in DMSO. The final top concentration was 10 μM with a 10 point 3-fold dilution in duplicate in the MultiTox assay.

20 μL was transferred from the 5×tet plate to the ARP and mixed, then 5 μL of 5×tet and the compounds were transferred to the cell plate and mixed using the Bravo. The cell plate was placed in the humidified 37° C. 5% CO₂ incubator for 24 hours.

The MultiTox-Fluor Multiplex Cytotoxicity Assay was performed in accordance with the manufacturer's protocol. After cells were incubated with tet and compound for 24 hours, 25 μL of 1×MultiTox reagent was added to each well using the Multidrop dispenser; the plates were placed on a plate shaker (600 rpm) for 2 minutes, then centrifuged briefly and placed back in the 37° C. incubator for 30 minutes. The cell viability (excitation: 400 nm, emission: 486 nm) and cytotoxicity (excitation: 485 nm, emission: 535 nm) were read using the EnVision plate reader. A ratio of dead (cytotoxicity) to live (viability) cells was reported. Data was exported and analyzed in Genedata. Data was normalized using percent of control, no tet (100% viability), and 50 ng/mL tet treated (0% viability), and fit using Smart Fit settings in Genedata.

### Potency Data for Compounds 1 to 78

The compounds of Formula I are useful as inhibitors of APOL1 activity. Table 8 below illustrates the IC₅₀ of Compounds 1 to 78 using procedures described above. The procedures above may also be used to determine the potency of any compounds of Formula I. In Table 8 below, the following meanings apply. For IP₅₀ (i.e., IC₅₀ for cell proliferation), “+++” means <0.1 μM; “++” means 0.1-0.5 μM; “+” means >0.5-1.0 μM.

TABLE 8
Potency Data for Compounds 1 to 78

| Compound | |
|---|---|
| No. | IP₅₀ (nM) |
| 1 | ++ |
| 2 | ++ |
| 3 | ++ |
| 4 | ++ |
| 5 | +++ |
| 6 | +++ |
| 7 | ++ |
| 8 | ++ |
| 9 | ++ |
| 10 | ++ |
| 11 | + |
| 12 | + |
| 13 | + |
| 14 | + |
| 15 | + |
| 16 | + |
| 17 | + |
| 18 | + |
| 19 | + |
| 20 | + |
| 21 | ++ |
| 22 | +++ |
| 23 | ++ |
| 24 | +++ |
| 25 | ++ |
| 26 | +++ |
| 27 | +++ |
| 28 | ++ |
| 29 | + |
| 30 | + |
| 31 | ++ |
| 32 | ++ |
| 33 | +++ |
| 34 | ++ |
| 35 | ++ |
| 36 | ++ |
| 37 | + |
| 38 | ++ |
| 39 | + |
| 40 | +++ |
| 41 | ++ |
| 42 | + |
| 43 | ++ |
| 44 | + |
| 45 | +++ |
| 46 | ++ |
| 47 | + |
| 48 | ++ |
| 49 | +++ |
| 50 | ++ |
| 51 | +++ |
| 52 | ++ |
| 53 | +++ |
| 54 | +++ |
| 55 | ++ |
| 56 | +++ |
| 57 | +++ |
| 58 | ++ |
| 59 | ++ |
| 60 | ++ |
| 61 | ++ |
| 62 | ++ |
| 63 | ++ |
| 64 | ++ |
| 65 | ++ |
| 66 | ++ |
| 67 | ++ |
| 68 | ++ |
| 69 | ++ |
| 70 | ++ |
| 71 | ++ |
| 72 | + |
| 73 | + |
| 74 | + |
| 75 | + |
| 76 | + |
| 77 | + |
| 78 | + |

### OTHER EMBODIMENTS

This disclosure provides merely non-limiting example embodiments of the disclosed subject matter. One skilled in the art will readily recognize from the disclosure and claims, that various changes, modifications and variations can be made therein without departing from the spirit and scope of the disclosure as defined in the following claims.

1. A compound represented by the following structural formula: 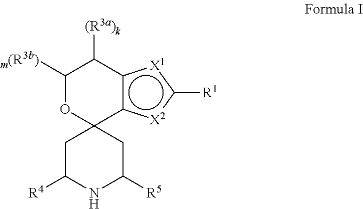
a tautomer thereof, a deuterated derivative of that compound or tautomer, or a pharmaceutically acceptable salt of any of the foregoing, wherein:
X¹ is chosen from S and —CR^{2a} and X² is chosen from S and —CR^{2b}, wherein:
one of X¹ and X² is S;
when X¹ is S, then X² is —CR^{2b}; and
when X² is S, then X¹ is —CR^{2a};
R¹ is chosen from hydrogen, halogen, cyano, —OH, C₁-C₆ alkyl, C₁-C₆ alkoxy, C₃-C₆ cycloalkyl, 5- to 8-membered heterocyclyl, and phenyl, wherein:
the C₁-C₆ alkyl of R¹ is optionally substituted with 1 to 3 groups independently chosen from halogen, cyano, 5- to 8-membered heterocyclyl (optionally substituted with 1 to 3 halogen groups), —OH, —NH₂, —NH(C₁-C₄ alkyl), —N(C₁-C₄ alkyl)₂, and C₁-C₄ alkoxy (optionally substituted with 1 to 3 halogen groups);
the C₁-C₆ alkoxy of R¹ is optionally substituted with 1 to 3 groups independently chosen from halogen;
the C₃-C₆ cycloalkyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂; and
the phenyl of R¹ is optionally substituted with 1 to 3 groups independently chosen from halogen, cyano, —OH, —NH₂, —NH(C₁-C₄ alkyl), —N(C₁-C₄ alkyl)₂, C₁-C₄ alkyl, C₁-C₄ alkoxy, —C(═O)NH₂, —C(═O)NH(C₁-C₄ alkyl), and —C(═O)N(C₁-C₄ alkyl)₂;
R^{2a} is chosen from hydrogen, halogen, cyano, —OH, oxo, and C₁-C₆ alkyl, wherein:
the C₁-C₆ alkyl of R^{2a} is optionally substituted with 1 to 3 groups independently chosen from halogen, cyano, —OH, and C₁-C₄ alkoxy;
R^{2b} is chosen from hydrogen, halogen, cyano, —OH, oxo, and C₁-C₆ alkyl;
each R^{3a} is independently chosen from halogen, cyano, —OH, C₁-C₆ alkoxy, and C₁-C₆ alkyl (optionally substituted with 1 to 3 groups independently chosen from halogen, cyano, and —OH); or
two R^{3a} together form an oxo group;
each R^{3b} is independently chosen from C₁-C₂ alkyl (optionally substituted with 1 to 3 groups independently chosen from halogen, cyano, and —OH); or
two R^{3a} together form an oxo group;
one of R⁴ and R⁵ is hydrogen and the other is chosen from C₁-C₆ alkyl, —C(═O)NH₂, —C(═O)O(C₁-C₄ alkyl), C₂-C₆ alkynyl, and 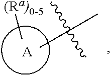
wherein:
the C₁-C₆ alkyl of R⁴ or R⁵ optionally substituted with 1 to 3 groups independently chosen from halogen, cyano, —OH, —NH₂, —NH(C₁-C₄ alkyl), —N(C₁-C₄ alkyl)₂, C₁-C₄ alkoxy, —C(═O)NH₂, —C(═O)NH(C₁-C₄ alkyl), —C(═O)N(C₁-C₄ alkyl)₂, C₃-C₆ cycloalkyl, 5 to 10-membered heterocyclyl, phenyl, and 5 to 10-membered heteroaryl;
Ring A is chosen from C₃-C₁₂ cycloalkyl, 3- to 12-membered heterocyclyl, C₆ and C₁₀ aryl, and 5- to 10-membered heteroaryl, wherein Ring A is optionally substituted with 1, 2, 3, 4, or 5 R^{a} groups, wherein:
R^{a}, for each occurrence, is independently chosen from halogen, cyano, C₁-C₆ alkyl, C₂-C₆ alkenyl, C₁-C₆ alkoxy, C₁-C₆ haloalkyl, C₁-C₆ haloalkenyl, C₁-C₆ haloalkoxy, —C(═O)NR^{h}Rⁱ, —NR^{h}Rⁱ, —NR^{h}C(═O)R^{k}, —NR^{h}(═O)OR^{k}, —NR^{h}C(═O)NRⁱRⁱ, —NR^{h}S(═O)ₚR^{k}, —OR^{k}, —OC(═O)R^{k}, —OC(═O)OR^{k}, —OC(═O)NR^{h}Rⁱ, —[O(CH₂)_{q}]ᵣO(C₁-C₆ alkyl), —S(═O)ₚR^{k}, —S(═O)ₚNR^{h}Rⁱ, —C(═O)OR^{k}, C₃-C₁₂ cycloalkyl, 3- to 12-membered heterocyclyl, C₆ and C₁₀ aryl, and 5- to 10-membered heteroaryl, wherein:
the C₁-C₆ alkyl, the C₁-C₆ alkoxy, the C₁-C₆ haloalkyl, and the C₂-C₆ alkenyl of R^{a} are each optionally substituted with 1 to 3 groups independently chosen from C₆ to C₁₀ aryl (optionally substituted with 1 to 3 R^{m} groups), 5- to 10-membered heterocyclyl (optionally substituted with 1 to 3 R^{m} groups), 5- to 10-membered heteroaryl (optionally substituted with 1 to 3 R^{m} groups), cyano,
—C(═O)R^{k}, —C(═O)OR^{k}, —C(═O)NR^{h}Rⁱ, —NR^{h}Rⁱ, —NR^{h}C(═O)R^{k}, —NR^{h}C(═O)OR^{k},
—NR^{h}C(═O)NRⁱRⁱ, —NR^{h}S(═O)ₚR^{k}, —OR^{k}, —OC(═O)R^{k}, —OC(═O)OR^{k}, —OC(═O)NR^{h}Rⁱ, —S(O)ₚR^{k}, —S(═O)ₚNR^{h}Rⁱ, and C₃-C₆ cycloalkyl (optionally substituted with 1 to 3 R^{m} groups);
the C₃-C₁₂ cycloalkyl, the 3 to 12-membered heterocyclyl, the C₆ and C₁₀ aryl, and the 5 to 10-membered heteroaryl of R^{a} are each optionally substituted with 1 to 3 groups independently chosen from halogen, cyano, C₁-C₄ alkyl, —C(═O)NR^{h}Rⁱ, —NR^{h}Rⁱ, —OR^{k}, and oxo, wherein:
R^{h}, Rⁱ, and R^{j}, for each occurrence, are each independently chosen from hydrogen, C₁-C₄ alkyl, C₆-C₁₀ aryl, and C₃-C₆ cycloalkyl, wherein:
the C₁-C₄ alkyl of any one of R^{h}, R¹, and Rⁱ is optionally substituted with 1 to 3 groups independently chosen from halogen, cyano, and —OH;
R^{k}, for each occurrence, is independently chosen from hydrogen, C₁-C₄ alkyl, 5- to 10-membered heterocyclyl, and C₃-C₆ carbocycles, wherein:
the C₁-C₄ alkyl of any one of R^{k} is optionally substituted with 1 to 3 groups independently chosen from halogen, cyano, and —OH;
R^{m}, for each occurrence, is independently chosen from halogen, cyano, oxo, C₁-C₆ alkyl, C₁-C₆ alkoxy, —S(═O)ₚR^{k}, and —OR^{k}, wherein:
the C₁-C₆ alkyl of R^{m} is optionally substituted with 1 to 3 groups independently chosen from halogen, cyano, and —OH;
k is an integer chosen from 0, 1, and 2, wherein, when R^{3a} is oxo, k is 1;
m is an integer chosen from 0, 1, and 2, wherein, when R³bis oxo, m is 1;
p, for each occurrence, is an integer chosen from 1 and 2; and
q and r, for each occurrence, is an integer independently chosen from 1, 2, 3, and 4.

2. The compound, tautomer, deuterated derivative, or pharmaceutically acceptable salt according to claim 1, wherein:
X¹ is chosen from S and —CR^{2a} and X² is chosen from S and —CR^{2b}, wherein:
one of X¹ and X² is S;
when X¹ is S, then X² is —CR^{2b}; and
when X² is S, then X¹ is —CR^{2a};
R¹ is chosen from halogen, C₁-C₆ alkyl, and C₃-C₆ cycloalkyl, wherein:
the C₁-C₆ alkyl of R¹ is optionally substituted with 1 to 3 groups independently chosen from halogen; and
the C₃-C₆ cycloalkyl of R¹ is optionally substituted with 1 to 3 groups independently chosen from halogen;
R^{2a} is chosen from hydrogen and C₁-C₆ alkyl, wherein:
the C₁-C₆ alkyl of R^{2a} is optionally substituted with 1 to 3 —OH groups;
R^{2b} is hydrogen;
each R^{3a} is independently chosen from —OH, C₁-C₆ alkoxy, and C₁-C₆ alkyl (optionally substituted with 1 to 3 groups independently chosen from halogen); or
two R^{3a} together form an oxo group;
each R^{3b} is independently chosen from C₁-C₂ alkyl (optionally substituted with 1 to 3 groups independently chosen from halogen, cyano, and —OH); or
two R^{3b} together form an oxo group;
one of R⁴ and R⁵ is hydrogen and the other is chosen from 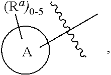
wherein:
Ring A is chosen from C₃-C₁₂ cycloalkyl, C₆ aryl, and 5- to 10-membered heteroaryl, wherein Ring A is optionally substituted with 1, 2, or 3 R^{a} groups, wherein:
R^{a}, for each occurrence, is independently chosen from halogen, C₁-C₆ alkyl, cycloalkyl —C(═O)NR^{h}Rⁱ, —OR^{k}, 3- to 12-membered heterocyclyl, C₆ aryl, and 5- to 10-membered heteroaryl, wherein:
the C₁-C₆ alkyl of R^{a} are each optionally substituted with 1 to 3 groups independently chosen from
—OR^{k} and
—S(═O)ₚNR^{h}Rⁱ;
the C₃-C₁₂ cycloalkyl, the 3- to 12-membered heterocyclyl, the C₆aryl, and the 5- to 10-membered heteroaryl of R^{a} are each optionally substituted with 1 to 3 groups independently chosen from halogen, C₁-C₄ alkyl,
—C(═O)NR^{h}Rⁱ, —OR^{k}, and oxo, wherein:
R^{h}, Rⁱ, and R^{j}, for each occurrence, are each independently chosen from hydrogen and C₁-C₄ alkyl; and
R^{k}, for each occurrence, is independently chosen from hydrogen and C₁-C₄ alkyl;
k is an integer chosen from 0, 1, and 2, wherein, when R^{3a} is oxo, k is 1;
m is an integer chosen from 0, 1, and 2; and
p, for each occurrence, is an integer chosen from 1 and 2.

3. The compound, tautomer, deuterated derivative, or pharmaceutically acceptable salt according to claim 1 or 2, wherein:
X¹ is chosen from S and —CR^{2a} and X² is chosen from S and —CR^{2b}, wherein:
one of X¹ and X² is S;
when X¹ is S, then X² is —CR^{2b}; and
when X² is S, then X¹ is —CR^{2a};
R¹ is chosen from halogen, C₁-C₆ alkyl, and C₃-C₆ cycloalkyl, wherein:
the C₁-C₆ alkyl of R¹ is optionally substituted with 1 to 3 groups independently chosen from halogen; and
the C₃-C₆ cycloalkyl of R¹ is optionally substituted with 1 or 2 groups independently chosen from halogen;
R^{2a} is chosen from hydrogen and C₁-C₆ alkyl, wherein:
the C₁-C₆ alkyl of R^{2a} is optionally substituted with 1 —OH group;
R^{2b} is hydrogen;
R^{3a} is independently chosen from —OH, C₁-C₆ alkyl, C₁-C₆ alkoxy, and oxo, wherein:
the C₁-C₆ alkyl of R^{3a} is optionally substituted with 1 to 3 groups independently chosen from halogen;
R^{3b} is chosen from C₁-C₂ alkyl;
-̅-̅-̅-̅-̅-̅, for each occurrence, is a single bond when R^{3a} is independently chosen from —OH and optionally substituted C₁-C₆ alkyl or when R³ is chosen from C₁-C₂ alkyl; or alternatively -̅-̅-̅-̅-̅-̅, for each occurrence, is a double bond when R^{3a} is oxo;
one of R⁴ and R⁵ is hydrogen and the other is chosen from 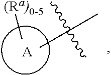
wherein:
Ring A is chosen from C₃-C₁₂ cycloalkyl, C₆ aryl, and 5- to 10-membered heteroaryl, wherein Ring A is optionally substituted with 1, 2, or 3 R^{a} groups, wherein:
R^{a}, for each occurrence, is independently chosen from halogen, C₁-C₆ alkyl, C₆ aryl, and 5- to 10-membered heteroaryl, wherein:
the C₁-C₆ alkyl of R^{a} are each optionally substituted with 1 to 3 groups independently chosen from —OR^{k} and —S(═O)ₚR^{k}; and
the C₆ aryl and the 5- to 10-membered heteroaryl of R^{a} are each optionally substituted with 1 to 3 groups independently chosen from halogen, C₁-C₄ alkyl, —C(═O)NR^{h}Rⁱ, —OR^{k}, and oxo, wherein:
R^{h}, Rⁱ, and R^{j}, for each occurrence, are each independently chosen from hydrogen and C₁-C₄ alkyl; and
R^{k}, for each occurrence, is independently chosen from hydrogen and C₁-C₄ alkyl;
k is an integer chosen from 0, 1, and 2, wherein, when R^{3a} is oxo, k is 1;
m is an integer chosen from 0, 1, and 2; and
p, for each occurrence, is an integer chosen from 1 and 2.

4. The compound, tautomer, deuterated derivative, or pharmaceutically acceptable salt according to claim 1, wherein the compound is represented by one of the following structural formulae: 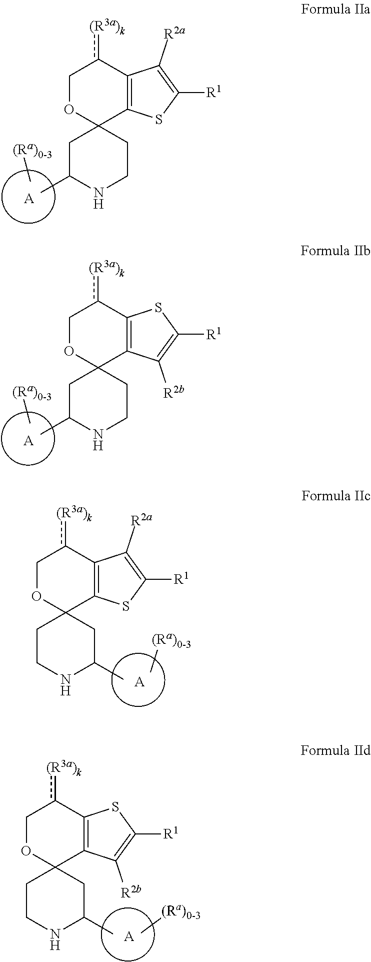
a tautomer thereof, a deuterated derivative of that compound or tautomer, or a pharmaceutically acceptable salt of any of the foregoing, wherein:
Ring A, for each occurrence, is chosen from C₃-C₆ cycloalkyl, phenyl, and 5- to 10-membered heteroaryl, each of which is optionally substituted with 1, 2, or 3 R^{a} groups; and
all other variables not specifically defined herein are as defined in any one of claims 1 to 3.

5. The compound, tautomer, deuterated derivative, or pharmaceutically acceptable salt according to claim 1, wherein the compound is represented by one of the following structural formulae: 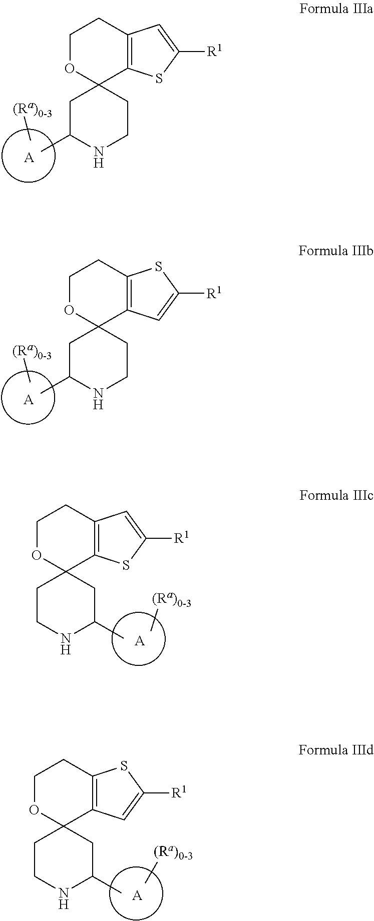
a tautomer thereof, a deuterated derivative of that compound or tautomer, or a pharmaceutically acceptable salt of any of the foregoing, wherein:
Ring A, for each occurrence, is chosen from C₃-C₆ cycloalkyl, phenyl, and 5- to 10-membered heteroaryl, each of which is optionally substituted with 1, 2, or 3 R^{a} groups; and
all other variables not specifically defined herein are as defined in any one of claims 1 to 3.

6. The compound, tautomer, deuterated derivative, or pharmaceutically acceptable salt according to any one of claims 1 to 5, wherein Ring A is chosen from 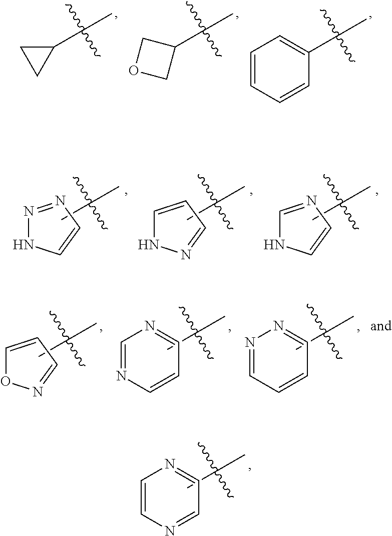
each of which is optionally substituted with 1, 2, or 3 R^{a} groups.

7. The compound, tautomer, deuterated derivative, or pharmaceutically acceptable salt according to any one of claims 1 to 5, wherein Ring A is chosen from 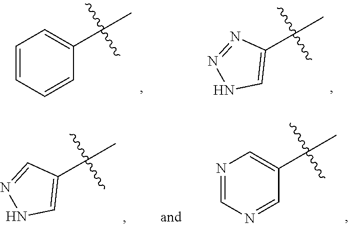 [SMILES: *c1ccccc1 ; *c1c[nH]nn1 ; *c1cn[nH]c1 ; *c1cncnc1]
each of which is optionally substituted with 1, 2, or 3 R^{a} groups.

8. A pharmaceutical composition comprising a compound according to any one of claims 1 to 7.

9. A method of treating a disease mediated by ApoL1, comprising administering a compound according to any one of claims 1 to 7 or a pharmaceutical composition according to claim 8.

10. The method of treating focal segmental glomerulosclerosis (FSGS),comprising administering a compound according to any one of claims 1 to 7 or a pharmaceutical composition according to claim 8.

11. The method of treating non-diabetic kidney disease (NDKD), comprising administering a compound according to any one of claims 1 to 7 or a pharmaceutical composition according to claim 8.

12. The method of treating cancer mediated by ApoL1, comprising administering a compound according to any one of claims 1 to 7 or a pharmaceutical composition according to claim 8.

13. The method of treating cancer according to claim 12, wherein the cancer is pancreatic cancer.

14. The method of treating according to any one of claims 9 to 13, wherein the patient to be treated possesses an APOL1 genetic variants.

15. The method of treating according to claim 14, wherein the genetic variant is chosen from G1: S342G:I384M and G2: N388del:Y389del.

16. A method of inhibiting APOL1 activity comprising contacting said APOL1 with at least one compound, tautomer, deuterated derivative, or pharmaceutically acceptable salt according to any one of claims 1 to 7, or a pharmaceutical composition according to claim 8.

17. Use of a compound according to any one of claims 1 to 7 in the manufacture of a medicament for the treatment of an ApoL1 mediated disease.

18. Use of a compound according to any one of claims 1 to 7 in the manufacture of a medicament for the treatment of FSGS.

19. Use of a compound according to any one of claims 1 to 7 in the manufacture of a medicament for the treatment of NDKD.

20. Use of a compound according to any one of claims 1 to 7 in the manufacture of a medicament for the treatment of cancer mediated by ApoL1.

21. Use of a compound according to any one of claims 1 to 7 in the manufacture of a medicament for the treatment of pancreatic cancer mediated by ApoL1.

22. Use of a compound according to any one of claims 1 to 7 in the manufacture of a medicament for inhibiting the activity of ApoL1 in a patient in need thereof.

23. A compound according to any one of claims 1 to 7, or a pharmaceutical composition according to claim 8, for use in inhibiting the activity of ApoL1 in a patient in need thereof.

24. A compound according to any one of claims 1 to 7, or a pharmaceutical composition according to claim 8, for use in treating an ApoL1 mediated disorder.

25. A compound according to any one of claims 1 to 7, or a pharmaceutical composition according to claim 8, for use in treating FSGS.

26. A compound according to any one of claims 1 to 7, or a pharmaceutical composition according to claim 8, for use in treating NDKD.

27. A compound according to any one of claims 1 to 7, or a pharmaceutical composition according to claim 8, for use in treating cancer mediated by ApoL1.

28. A compound according to any one of claims 1 to 7, or a pharmaceutical composition according to claim 8, for use in treating pancreatic cancer mediated by ApoL1.
